(19) United States
(12) Patent Application Publication
(10) Pub. No.: US 2025/0346893 A1
(43) Pub. Date: Nov. 13, 2025

(54) SYNTHETIC INTRONS FOR TARGETED GENE EXPRESSION

(71) Applicants: Fred Hutchinson Cancer Center, Seattle, WA (US); Memorial Sloan Kettering Cancer Center, New York, NY (US)

(72) Inventors: Robert K. Bradley, Seattle, WA (US); Omar Abdel-Wahab, New York, NY (US); Robert Stanley, New York, NY (US); Bo Liu, New York, NY (US); Emma De Neef, Seattle, WA (US)

(73) Assignees: Fred Hutchinson Cancer Center, Seattle, WA (US); Memorial Sloan Kettering Cancer Center, New York, NY (US)

(21) Appl. No.: 18/702,893

(22) PCT Filed: Oct. 24, 2022
(86) PCT No.: PCT/US2022/078615

§ 371 Date: Apr. 19, 2024

Related U.S. Application Data
(60) Provisional application No. 63/270,981, filed on Oct. 22, 2021.

(51) Int. Cl. C12N 15/113; A61K 48/00; A61P 35/02; C12N 15/85

(52) U.S. Cl. CPC C12N 15/113; A61P 35/02; C12N 15/85; A61K 48/00; C12N 2320/33

(57) ABSTRACT

The disclosure provides artificial nucleic acid introns configured for selective splicing in cells with aberrant RNA splicing activity, e.g., neoplastic cells. The artificial intron can comprise an upstream flanking exon, an upstream intron, an alternatively spliced “cassette” exon, a downstream intron, and a downstream flanking exon. Also provided are constructs integrating the artificial introns with exons in a configuration that, when the artificial intron is spliced out by the aberrant RNA splicing factors, encode a functional protein. Also disclosed are methods that employ the disclosed platform of selective expression, including, targeted gene therapy methods (e.g., in cancers), diagnostics and imaging, and drug screening.

### CROSS-REFERENCES TO RELATED APPLICATIONS

This application claims the benefit of U.S. Provisional Application Nos. 63/270,981, filed Oct. 22, 2021, the disclosure of which is hereby expressly incorporated herein by reference in its entirety.

### STATEMENT OF GOVERNMENT LICENSE RIGHTS

This invention was made with Government support under HL128239, DK103854, and CA251138 awarded by the National Institutes of Health. The Government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 1896-P64WO_Seq_List_20221024_ST26. The XML file is 71 KB; was created on Oct. 24, 2022; and is being submitted via Patent Center with the filing of the specification.

### BACKGROUND

Gene therapy, i.e., the introduction of novel genetic material into cells, has promise as a powerful modality for the treatment of cancers. See, e.g., Amer, M., Gene therapy for cancer: present status and future perspective, Mol. Cell Ther. 2:27 (2014). Unfortunately, existing strategies have not achieved the desired clinical benefits. One major challenge for developing gene therapy for cancer treatment is that accidental delivery of the gene therapy payload to healthy normal cells can result in unintended and adverse side effects. For example, if the payload was a “killer gene” that triggered cancer cell apoptosis, then delivery of this payload to healthy cells could result in their unwanted deaths leading to potentially severe side-effects. As a consequence, developing a reliable and generalizable method to permit expression of a given gene or protein in cancer cells, but not normal cells, or alternately in normal cells but not cancer cells, would be a major and important step toward bringing gene therapy for cancers into the clinic. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides an artificial nucleic acid construct comprising an intron. The intron comprises:

In some embodiments, the intron is at least about 20 nucleotides to about 1000 nucleotides in length.

In some embodiments, the intron comprises domains derived from a human wildtype intron selected from intron 10 of MELK, exon 10 of MELK, and intron 11 of MELK; intron 34 of GTF3C1; intron 4 of INTS3 and exon 5 of INTS3; and exon 3 of ZNF19 and exon 4 of ZNF 19. In some embodiments, the human wildtype intron from which the intron is derived is one of the following: intron 10 of MELK comprising a sequence set forth in SEQ ID NO:22; exon 10 of MELK comprising a sequence set forth in SEQ ID NO:23; intron 11 of MELK comprising a sequence set forth in SEQ ID NO:24; intron 12 of MELK comprising a sequence set forth in SEQ ID NO:1; exon 12 of MELK comprising a sequence set for in SEQ ID NO:2; intron 13 of MELK comprising a sequence in SEQ ID NO:3; intron 34 of GTF3C1 comprising a sequence set forth as SEQ ID NO:25; intron 1 of ARFIP2 comprising a sequence set forth in SEQ ID NO:26; intron 4 of INTS3 comprising a sequence set forth in SEQ ID NO:27; exon 5 of INTS3 comprising a sequence set forth in SEQ ID NO:28; exon 3 of ZNF19 comprising a sequence set forth in SEQ ID NO: 29; intron 3 of ZNF19 comprising a sequence set forth in SEQ ID NO:30; and exon 4 of ZNF19 comprising a sequence set forth in SEQ ID NO:31, and wherein the synthetic intron further comprises one, two, three, or more of the following features: a 5′ splice site comprising a GT dinucleotide immediately followed by a consensus 5′ splice site context, optionally wherein the consensus 5′ splice site context includes one of AAG, GAG, GTG, and the like; a canonical 3′ splice site comprising an AG dinucleotide immediately preceded by a C or T; at least one alternatively spliced cassette exon embedded within the synthetic intron; at least one cryptic 5′ splice site, located at least 5 nucleotides upstream or downstream of the canonical 5′ splice site, with a GT dinucleotide and comprising a sequence that is a weaker 5′ splice site than is the canonical 5′ splice site, where splice site strength is estimated with the MaxEntScan algorithm or similar method; at least one cryptic 3′ splice site, located at least 5 nucleotides upstream or downstream of the canonical 3′ splice site, with an AG dinucleotide and comprising a sequence that is a weaker 3′ splice site than is the canonical 3′ splice site, where splice site strength is estimated with the MaxEntScan algorithm or similar methods. In some embodiments, the intron has a 5′ end domain with about 10 to about 150 nucleotides having at least 50% sequence identity to a sequence of the 5′-most 10 to about 150 nucleotides of the wildtype intron. In some embodiments, the intron has a 3′ end domain with about 50 to about 350 nucleotides having at least 50% sequence identity to a sequence of the 3′-most 50 to about 350 nucleotides of the wildtype intron. In some embodiments, the intron has a sequence with at least 75% sequence identity to a selected sequence.

In some embodiments, the canonical 5′ splice site comprises a sequence selected from GTGAG, GTAAG, GTGCG, GTACG, GTGGG, GTAGG, GTGTG, GTATG, and GTATC. In some embodiments, the at least one cryptic 5′ splice site comprises a sequence selected from GTA, GTC, GTG, and GTT. In some embodiments, the intron comprises a plurality of cryptic 5′ splice sites within about 100 nucleotides upstream of the canonical 5′ splice site or within about 100 nucleotides downstream of the canonical 5′ splice site, and wherein each of the plurality of the cryptic 5′ splice sites comprises a sequence independently selected from GTA, GTC, GTG, and GTT. In some embodiments, the at least one alternatively spliced cassette exon comprises a sequence flanked by the dinucleotides AG and GT. In some embodiments, the canonical 3′ splice site comprises a sequence selected from AAG, CAG, and TAG. In some embodiments, the at least one cryptic 3′ splice site comprises a sequence selected from AAG, CAG, GAG, and TAG. In some embodiments, the intron comprises a plurality of cryptic 3′ splice sites within about 100 nucleotides upstream of the canonical 3′ splice site or within about 100 nucleotides downstream of the canonical 3′ splice site, and wherein each of the plurality of the cryptic 3′ splice sites comprises a sequence independently selected from AAG, CAG, GAG, and TAG.

In some embodiments, the intron is configured to be spliced differently in a cancer cell comprising a change-of-function or loss-of-function mutation in a recurrently mutated RNA splicing factor gene relative to the splicing pattern of the intron in a cell lacking a change-of-function or loss-of-function mutation in a recurrently mutated RNA splicing factor gene. In some embodiments, the RNA splicing factor gene is SRSF2.

In some embodiments, the nucleic acid construct further comprises a first exon domain and a second exon domain, wherein the intron is disposed between the first exon domain and the second exon domain. In some embodiments, the combination of the first exon domain and the second exon domain without the intron encodes part or all of a protein of interest. In some embodiments, the nucleic acid intron construct comprises an expression cassette comprising the first exon domain, the intron, the second exon domain, and a promoter sequence operatively linked thereto. In certain embodiments an alternatively, or differentially recognized, spliced cassette exon is embedded within surrounding introns.

In another aspect, the disclosure provides a method of modifying a nucleic acid sequence to permit selective expression, or alternately selective lack of expression, in a cell characterized by a mutation in an RNA splicing factor gene. The method comprises: (1) providing a sequence of a target nucleic acid molecule and sequence of an artificial nucleic acid intron as described herein, wherein the artificial nucleic acid intron is derived from a wildtype intron with known nucleotide sequences of upstream and downstream flanking exons; (2) identifying one or more dinucleotides in the target nucleic acid sequence that are identical to an intron dinucleotide sequence consisting of the 3′-most nucleotide of the upstream exon flanking the wildtype intron and the 5′-most nucleotide of the downstream exon flanking the wildtype intron; (3) selecting a dinucleotide identified in step (2) as an insertion point, wherein the insertion point divides the target nucleic acid into a first domain and a second domain, optionally wherein one of the first domain and second domain is at least about 50% of the length of the other of the first domain and second domain; and (4) inserting an artificial intron molecule with the artificial nucleic acid intron sequence between the first domain and the second domain of the target nucleic acid molecule. In some embodiments, step (3) further comprises: computationally inserting the sequence of the artificial nucleic acid intron at the selected insertion point to create a hypothetical exonic flanking sequence context for a 5′-most 5′ splice site and a 3′-most 3′ splice site; computing strength scores for the 5′-most 5′ splice site and the 3′-most 3′ splice site, respectively, in their hypothetical exonic contexts; comparing the computed strength scores for the 5′-most 5′ splice site and 3′-most 3′ splice site within their hypothetical exonic contexts to strength scores of the respective 5′ splice site and 3′-most 3′ splice site of the wildtype intron in its wildtype exonic context from which the artificial nucleic acid intron is derived; and selecting a dinucleotide wherein computational insertion of the artificial nucleic acid intron sequence results in strength scores for the 5′-most 5′ splice site and 3′-most 3′ splice site in their hypothetical exonic contexts that differ by about 50% or less of the respective 5′ splice site and 3′-most 3′ splice site scores of the wildtype intron in its wildtype exonic context. In some embodiments, strength scores are computed with a standard method such as MaxEntScan::scores5ss, MaxEntScan::score3ss, HumanSplicingFinder, and other similar algorithms.

In some embodiments, the method further comprises introducing one or more synonymous codon mutations into the nucleic acid that improve or weaken one or both scores for the 5′-most 5′ splice site and/or 3′-most 3′ splice site in their hypothetical exonic contexts.

In some embodiments, the method further comprises introducing one or more synonymous codon mutations into the nucleic acid that result in creation of one or more exonic splicing enhancers. In some embodiments, the one or more exonic splicing enhancers is/are selected from CCNG, CGNG, GCNG, and GGNG, where N is any nucleotide, and other sequences with enhanced likelihood of binding by serine/arginine-rich (SR) proteins.

In some embodiments, the method further comprises introducing one or more synonymous codon mutations into the nucleic acid that result in creation of one or more exonic splicing silencers. In some embodiments, the one or more exonic splicing silencers is/are selected from TTTGTTCCGT (SEQ ID NO:32), GGGTGGTTTA (SEQ ID NO:33), GTAGGTAGGT (SEQ ID NO:34), TTCGTTCTGC (SEQ ID NO:35), GGTAAGTAGG (SEQ ID NO:36), GGTTAGTTTA (SEQ ID NO:37), TTCGTAGGTA (SEQ ID NO:38), GGTCCACTAG (SEQ ID NO:39), TTCTGTTCCT (SEQ ID NO:40), TCGTTCCTTA (SEQ ID NO:41), GGGATGGGGT (SEQ ID NO:42), GTTTGGGGGT (SEQ ID NO:43), TATAGGGGGG (SEQ ID NO:44), GGGGTTGGGA (SEQ ID NO:45), TTTCCTGATG (SEQ ID NO:46), TGTTTAGTTA (SEQ ID NO:47), TTCTTAGTTA (SEQ ID NO:48), GTAGGTTTG, GTTAGGTATA (SEQ ID NO:49), TAATAGTTTA (SEQ ID NO:50), TTCGTTTGGG (SEQ ID NO:51), and the like, or sequences with at least 50% identity thereto.

In some embodiments, two or more artificial intron molecules are inserted into the target nucleic acid resulting in a plurality of domains, optionally wherein each of the plurality of domains is at least about 50% of the length of the other domain(s). In some embodiments, the target nucleic acid molecule is an isolated nucleic acid molecule with a protein-coding sequence (CDS) that encodes a protein of interest, and the modified target nucleic acid molecule is configured to permit selective expression, or alternately selective lack of expression, in a cell characterized by a mutation in an RNA splicing factor gene.

In some embodiments, the method further comprises introducing the modified target nucleic acid molecule to a cancer cell with a mutation in an RNA splicing factor gene and permitting expression, or alternately selective lack of expression, of the protein of interest.

In some embodiments, the target nucleic acid molecule is a gene in the chromosome of a cell, wherein the gene encodes a protein of interest, and the modified target nucleic acid molecule is configured for selective expression, or alternately selective lack of expression, in a cell characterized by a mutation in an RNA splicing factor gene. In some embodiments, the cell is a cancer cell and the mutation in an RNA splicing factor gene is a change-of-function or loss-of-function mutation in a recurrently mutated RNA splicing factor gene; wherein the artificial intron sequence is configured to be spliced differently in a cancer cell comprising the change-of-function or loss-of-function mutation in the recurrently mutated RNA splicing factor gene, relative to the splicing pattern of the intron in a cell lacking the change-of-function or loss-of-function mutation in the recurrently mutated RNA splicing factor gene; wherein the different splicing pattern of the artificial intron sequence results in production of different mature transcripts of the modified target nucleic acid molecule in a cancer cell comprising the change-of-function or loss-of-function mutation in the recurrently mutated RNA splicing factor gene, relative to the splicing pattern of the intron in a cell lacking the change-of-function or loss-of-function mutation in the recurrently mutated RNA splicing factor gene; and wherein the production of different mature transcripts of the modified nucleic acid molecule permits either selective expression, or alternately selective lack of expression, of a desired protein from the target nucleic acid molecule in the cancer cell, and the opposite pattern in a cell lacking the change-of-function or loss-of-function mutation in the recurrently mutated RNA splicing factor gene.

In another aspect, the disclosure provides a method of selectively expressing, or alternately selectively not expressing, a gene of interest in a cell, wherein the cell comprises a change-of-function or loss-of-function mutation in a recurrently mutated RNA splicing factor gene. The method comprises: introducing to the cell an expression cassette comprising a coding sequence (CDS) interrupted by at least one artificial nucleic acid intron as described herein, wherein the expression cassette further comprises a promoter operatively linked to the CDS; and permitting transcription of the coding sequence and modified splicing of the transcript induced by the artificial nucleic acid intron in the resulting transcript in conjunction with the mutated splicing factor.

In some embodiments, the cell is a cancer cell and the mutation in an RNA splicing factor gene is a change-of-function or loss-of-function mutation in a recurrently mutated RNA splicing factor gene.

In some embodiments, the cancer is a myelodysplastic syndrome (MDS), chronic myelomonocytic leukemia (CMML), acute myeloid leukemia (AML), myeloproliferative neoplasms (MDN), uveal melanoma, bladder cancer, lung adenocarcinoma, or other neoplasm with recurrent SRSF2 mutations. In some embodiments, upon splicing of the at least one artificial nucleic acid intron from the gene transcript, the gene of interest encodes a functional therapeutic protein. In some embodiments, the functional therapeutic protein is a toxin, chemokine, cytokine, growth factor, targetable cell-surface protein, targetable antigen, druggable enzyme, detectable marker, and the like.

In another aspect, the disclosure provides a method of treating in a subject with cancer, wherein the cancer is characterized by a change-of-function or loss-of-function mutation in a recurrently mutated RNA splicing factor gene. The method comprises administering to the subject an effective amount of a therapeutic composition comprising an expression cassette comprising a coding sequence (CDS) interrupted by at least one artificial nucleic acid intron as described herein, wherein the expression cassette further comprises a promoter operatively linked to the CDS.

In some embodiments, the cancer is selected from a myelodysplastic syndrome (MDS), chronic myelomonocytic leukemia (CMML), myeloproliferative neoplasms (MDN), or acute myeloid leukemia (AML), uveal melanoma, bladder cancer, lung adenocarcinoma, and other neoplasm with recurrent SRSF2.

In some embodiments, upon splicing of the at least one artificial nucleic acid intron from the gene transcript in a cancer cell the CDS encodes a functional therapeutic protein. In some embodiments, the functional therapeutic protein is a toxin, chemokine, cytokine, growth factor, targetable cell-surface protein, targetable antigen, druggable enzyme, detectable marker, and the like. In some embodiments, the functional therapeutic protein is a chemokine, cytokine, or growth factor, and wherein the chemokine, cytokine, or growth factor stimulates an increased immune response against the cancer cell. In some embodiments, the functional therapeutic protein is IFNα, IFNβ, IFNγ, IL-2, IL-12, IL-15, IL-18, IL-24, TNFα, GM-CSF, and the like, or functional domains or derivatives thereof. In some embodiments, the functional therapeutic protein is a targetable cell-surface protein or targetable antigen, and the method further comprises administering to the subject an effective amount of a second therapeutic composition comprising an affinity reagent that specifically binds the antigen. In some embodiments, the targetable cell-surface protein or targetable antigen is CD19, CD22, CD23, CD123, ROR1, truncated EGFR (EGFRt), or functional domains thereof, and the like. In some embodiments, the second therapeutic composition comprises an antibody, or a fragment or derivative thereof, an immune cell expressing an antibody, or fragment or derivative thereof, or an immune cell expressing a T cell receptor, or fragment or derivative thereof, and wherein the antibody or T cell receptor, or fragment or derivative thereof, specifically binds the antigen. In some embodiments, the functional therapeutic protein is a toxin, wherein the toxin is optionally Caspase 9, TRAIL, Fas ligand, and the like, or functional fragments thereof. In some embodiments, the functional therapeutic protein is a druggable enzyme, optionally wherein: the druggable enzyme is herpes simplex virus thymidine kinase and the method further comprises administering to the subject an effective amount of ganciclovir; the druggable enzyme is cytosine deaminase and the method further comprises administering to the subject an effective amount of 5-fluorocytosine; the druggable enzyme is nitroreductase and the method further comprises administering to the subject an effective amount of CB1954 or analogs thereof; the druggable enzyme is carboxypeptidase G2 and the method further comprises administering to the subject an effective amount of CMDA, ZD-2767P, and the like; the druggable enzyme is purine nucleoside phosphorylase and the method further comprises administering to the subject an effective amount of 6-methylpurine deoxyriboside, and the like; the druggable enzyme is cytochrome P450 and the method further comprises administering to the subject an effective amount of cyclophosphamide, ifosfamide, and the like; the druggable enzyme is horseradish peroxidase and the method further comprises administering to the subject an effective amount of indole-3-acetic acid, and the like; or the druggable enzyme is carboxylesterase and the method further comprises administering to the subject an effective amount of irinotecan, and the like.

In some embodiments, the functional therapeutic protein is a detectable marker, and the method further comprises surgically removing the cancer cells expressing the detectable marker. In some embodiments, the expression cassette is disposed in a vector, optionally a viral vector, for intracellular delivery. In some embodiments, the viral vector is derived from AAV, adenovirus, herpes simplex virus, retrovirus, lentivirus, alphavirus, flavivirus, rhabdovirus, measles virus, Newcastle disease virus, Coxsackievirus, poxvirus, and the like.

In some embodiments, the therapeutic composition further comprises a vehicle for intracellular delivery and a pharmaceutically acceptable carrier. In some embodiments, the vehicle is a liposome, nanocapsule, nanoparticle, exosome, microparticle, microsphere, lipid particle, vesicle, and the like, configured for the introduction of the expression cassette into cancer cells.

In another aspect, the disclosure provides method of enhancing surgical resection of a tumor from a subject, wherein the tumor is characterized by a change-of-function or loss-of-function mutation in a recurrently mutated RNA splicing factor gene. The method comprises: administering to the subject an effective amount of a therapeutic composition comprising an expression cassette comprising a coding sequence (CDS) encoding a detectable marker, wherein the CDS is interrupted by at least one artificial nucleic acid intron as described herein, and wherein the expression cassette further comprises a promoter operatively linked to the CDS.

In some embodiments, the RNA splicing factor gene is SRSF2.

In some embodiments, the detectable marker is a fluorescent or luminescent protein. In some embodiments, the method further comprises detecting fluorescent or luminescent tumor cells and surgically resecting the fluorescent or luminescent tumor cells.

In some embodiments, the expression cassette is disposed in a vector, optionally a viral vector, for intracellular delivery. In some embodiments, the viral vector is derived from AAV, adenovirus, herpes simplex virus, retrovirus, lentivirus, alphavirus, flavivirus, rhabdovirus, measles virus, Newcastle disease virus, Coxsackievirus, poxvirus, and the like.

In some embodiments, the therapeutic composition further comprises a vehicle for intracellular delivery and a pharmaceutically acceptable carrier. In some embodiments, the vehicle is a liposome, nanocapsule, nanoparticle, exosome, microparticle, microsphere, lipid particle, vesicle, and the like, configured for the introduction of the expression cassette into cancer cells.

In another aspect, the disclosure provides a method of screening candidate compositions for activity in a cell, wherein the cell has a genetic background comprising a change-of-function or loss-of-function mutation in a recurrently mutated RNA splicing factor gene. The method comprises contacting the cell with an expression cassette comprising a coding sequence (CDS) interrupted by at least one artificial nucleic acid intron as described herein. The expression cassette further comprises a promoter operatively linked to the CDS, and wherein upon splicing of the artificial nucleic acid intron the CDS encodes or does not encode a detectable reporter protein. The specific splicing outcome depends upon mutant splicing factor activity in the cell. The method further comprises contacting the cell with a candidate composition; permitting transcription of the coding sequence; and detecting the presence or absence of a functional reporter protein.

In some embodiments, detection of a functional reporter protein or a relative increase of functional reporter protein in the cell indicates the candidate composition does not suppress activity of the mutated RNA splicing factor in the cell. Detection of an absence or relative reduction in functional reporter protein in the cell indicates the candidate composition does suppress activity of the mutated RNA splicing factor in the cell.

In some embodiments, detection of a functional reporter protein in the cell indicates the candidate composition suppresses activity of the mutated RNA splicing factor in the cell. An absence or relative reduction in detected functional reporter protein in the cell indicates the candidate composition does not suppress activity of the mutated RNA splicing factor in the cell.

In some embodiments, detecting the presence of a functional reporter protein comprises quantifying the amount of reporter protein. In some embodiments, the reporter protein is a fluorescent or luminescent protein.

In some embodiments, the method further comprises contacting a control cell without a change-of-function or loss-of-function mutation in a recurrently mutated RNA splicing factor gene with the expression cassette and further contacting the control cell with the candidate composition.

In some embodiments, the candidate composition is selected from a small molecule, protein (e.g., antibody, or fragment or derivative thereof, enzyme, and the like), and nucleic acid construct to alter the genome or transcriptome of the cell, or a complex of a nucleic acid and protein. In some embodiments, the nucleic acid construct is an interfering RNA construct. In some embodiments, the candidate composition comprises a guide nucleic acid specific for a target sequence and an associated nuclease that modifies and/or cleaves a nucleic acid molecule upon binding of the guide nucleic acid to its target sequence. In some embodiments, the candidate composition comprises a guide nucleic acid specific for a target sequence and an associated catalytically inactive nuclease, wherein binding of the guide nucleic acid to the target sequence results in modification of transcription, splicing, or translation of the target sequence. In some embodiments, the associated nuclease is Cas9, Cas12, Cas13, Cas14, variants thereof, and the like. In some embodiments, the candidate composition comprises a Transcription Activator-Like Effector Nuclease (TALEN), Zinc Finger Nuclease (ZFN), or recombinase fusion protein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1E illustrate the design of an artificial intron sequence configured for selective splicing in cells with mutated splicing factor, SRSF2. This functionality of the synthetic intron design is leveraged to create an expression cassette that can drive selective expression of a complete coding sequence in cells, e.g., cancer cells. FIG. 1A depicts an RNA-seq read coverage plot illustrating decreased inclusion of endogenous MELK exon 12 in K562 cells engineered to bear SRSF2P95H relative to isogenic WT K562 cells. FIG. 1B shows RT-PCR demonstrating higher levels of the endogenous MELK exon 12 exclusion isoform (or equivalently, decreased inclusion of the exon) in the illustrated cancer cells lines with or without the SRSF2P95H mutation. These data are complementary to those illustrated in FIG. 1A. FIG. 1C shows a schematic of an AAV construct containing the IL-2 CDS interrupted by the MELK synthetic intron. The MELK synthetic intron consists of three contiguous components: a 250 nt intron that is derived from intron 12 of the endogenous MELK gene (SEQ ID NO:1), an 85 nt alternatively spliced (“cassette”) exon that is derived from exon 12 of the endogenous MELK gene (SEQ ID NO:2), and a 250 nt intron that is derived from intron 13 of the endogenous MELK gene (SEQ ID NO:3). (Note that the “synthetic intron” refers to all three components together.) FIG. 1D Top shows RT-PCR demonstrating higher levels of the isoform encoding IL-2 protein produced in MARIMO cells expressing SRSF2P95H relative to WT cells following introduction of the construct illustrated in FIG. 1C. The isoform encoding IL-2 protein arises when the cassette exon within the synthetic intron construct is skipped (e.g., not included in the mature mRNA). FIG. 1D Bottom shows RT-PCR demonstrating splicing of the corresponding endogenous MELK region in these cells. FIG. 1E depicts the sequence of the MELK cassette exon (exon 12) within the synthetic intron construct (SEQ ID NO:2). GGNG (N=any nucleotide) sequence motifs, which have been shown to be associated with reduced exon inclusion in SRSF2-mutant cells due to reduced binding of mutant versus WT SRSF2 protein, are highlighted.

FIGS. 2A and 2B depict SRSF2 wildtype and mutant patient RNA-sequencing data from three independent studies showing intron retention events in the genes INTS3 (FIG. 2A) and ARFIP2 (FIG. 2B). Reference genome Hg19.

FIGS. 3A to 3D depict RNA-sequencing data from isogenic cells line K562 SRSF2^{+/+} and SRSF2^{+/P95H} showing alternative splicing events in the genes GTF3C1 (FIG. 3A), MELK (FIG. 3B), ARFIP2 (FIG. 3C), ZNF19 (FIG. 3D) and INTS3 (FIG. 3E).

FIGS. 4A to 4C depict RT-PCR results of endogenous splicing events in SRSF2^{+/+} and SRSF2^{+/P95H} cell lines. FIG. 4A depicts gel electrophoresis of RT-PCR products from SRSF2^{+/+} or SRSF2^{+/P95H} isogenic cell lines Marimo, OCI-AML2 and K562 amplified using primers for an endogenous alternative exon skipping even in the MELK gene. FIG. 4B depicts gel electrophoresis of RT-PCR products from SRSF2^{+/+} or SRSF2^{+/P95H} isogenic cell line K562 or TF-1 (SRSF2^{+/+}) and KO52 (SRSF2^{+/P95H}) amplified using primers for endogenous alternative splicing events in the genes GTF3C1 (5′ alternative splice site (ss)), ZNF19 (3′ alternative ss) and INTS3 (intron retention). FIG. 4C depicts gel electrophoresis of RT-PCR products from SRSF2^{+/+} or SRSF2+/P95H isogenic cell line K562 or TF-1 (SRSF2^{+/+}) and KO52 (SRSF2^{+/P95H}) amplified using primers for an endogenous alternative splicing event in the gene ARFIP2 (3′ alternative ss). The event of interest is highlighted by boxes.

FIGS. 5A and 5B depict RT-PCR results of splicing events in K562 cells transduced with MELK or GTF3C1 synthetic introns. FIG. 5A shows gel electrophoresis of RT-PCR products from SRSF2^{+/+} or SRSF2^{+/P95H} isogenic K562 cells amplified using primers for HSV-TK, endogenous MELK or endogenous EZH2 events. Cells utilized include non-transduced cells, cells with construct #487 (positive control), cells with construct #497 (negative control) and cells with GFP/HSV-TK MELK synthetic introns MELK 623nt, MELK 397nt, and MELK 249nt. FIG. 5B shows gel electrophoresis of RT-PCR products from SRSF2^{+/+} or SRSF2^{+/P95H} isogenic K562 cells amplified using primers for HSV-TK, endogenous MELK or endogenous EZH2 events. Cells utilized include non-transduced cells, cells with construct #487 (positive control), cells with construct #497 (negative control) and cells with GFP/HSV-TK GTF3C1 synthetic introns GTF3C1_325nt and GTF3C1_250nt.

FIGS. 6A to 6E depict the design schematics of various synthetic introns derived from endogenous alternative splicing events in MELK (FIG. 6A), GTF3C1 (FIG. 6B), ARFIP2 (FIG. 6C), INTS3 (FIG. 6D), and ZNF19 (FIG. 6E) genes.

FIGS. 7A to 7C depict schematics of additional vectors generated to study synthetic introns.

FIG. 8 depicts the schematic for a combination MELK and GTF3C1 synthetic intron design. The first 125 nt synthetic intron replaces the 5′ splice sequence of the MELK 249 nt synthetic intron just adjacent to the HSV coding sequence creating a 356 nt continuation synthetic intron.

FIGS. 9A and 9B depict flow cytometry of isogenic K562 cells transduced with retroviral Ef1a bichromatic synthetic intron MELK 623 nt. FIG. 9A shows flow cytometry plots of mCherry+K562 cells (left plot) that are dichotomized by mCherry and GFP expression (right plot) in K562 cells transduced with retroviral Ef1a bichromatic synthetic intron MELK 623 nt. FIG. 9B shows a GFP histogram plot of mCherry+K562 cells transduced with retroviral Ef1a bichromatic synthetic intron MELK 623 nt.

FIGS. 10A and 10B depict flow cytometry of isogenic K562 cells transduced with retroviral Ef1a bichromatic synthetic intron MELK 249 nt. FIG. 10A shows flow cytometry plots of mCherry+K562 cells (left plot) that are dichotomized by mCherry and GFP expression (right plot) in K562 cell lines transduced with retroviral Ef1a bichromatic synthetic intron MELK 249 nt.

FIGS. 11A and 11B depict flow cytometry of isogenic K562 cells transduced with retroviral Ef1a bichromatic synthetic intron GTF3C1_250 nt. FIG. 11A shows flow cytometry plots of mCherry+K562 cells (left plot) that are dichotomized by mCherry and GFP expression (right plot) in K562 cell lines transduced with retroviral Ef1a bichromatic synthetic intro GTF3C1_250 nt. FIG. 11B shows GFP histogram plot of mCherry+K562 cells transduced with retroviral Ef1a bichromatic synthetic intron GTF3C1_250 nt.

FIGS. 12A and 12B depict flow cytometry of isogenic K562 cells transduced with lentiviral PGK bichromatic synthetic intron MELK 249 nt. FIG. 12A shows flow cytometry plots of mCherry+K562 cells (left plot) that are dichotomized by mCherry and GFP expression (right plot) in K562 cell lines transduced with lentiviral bichromatic synthetic intron MELK 249 nt. FIG. 12B shows a GFP histogram plot of mCherry⁺ K562 cells transduced with lentiviral PGK bichromatic synthetic intron MELK 249 nt.

FIGS. 13A to 13C depicts a K562 ganciclovir in vitro killing assay. Relative viability of non-transduced (FIG. 13A), GFP/HSV-TK MELK 623 nt synthetic intron (FIG. 13B) or #497 negative control (FIG. 13C) K562 isogenic cell lines treated with increasing concentrations of ganciclovir (GCV) in vitro. Cell viability was analyzed at day 11 of plating utilizing a commercial cell viability assay (CellTiter-Glo® Luminescent Cell Viability Assay, ProMega). Data represents two independent experiments performed in triplicate.

FIGS. 14A to 14F depict a K562 ganciclovir in vitro killing assay. Relative viability of non-transduced (FIG. 14A), #497 negative control (FIG. 14B), or #487 positive control (FIG. 14C) K562 isogenic cell lines treated with increasing concentrations of ganciclovir (GCV) in vitro. Relative viability of GFP/HSV-TK MELK 397nt synthetic intron (FIG. 14D), GFP/HSV-TK MELK 249nt synthetic intron (FIG. 14E), or GFP/HSV-TK GTF3C1_250 nt synthetic intron (FIG. 14F) K562 isogenic cell lines treated with increasing concentrations of ganciclovir (GCV) in vitro. Cell viability was analyzed at day 7 of plating utilizing a commercial cell viability assay (CellTiter-Glo® Luminescent Cell Viability Assay, ProMega). Data represent two independent experiments performed in triplicate.

### DETAILED DESCRIPTION

Many cancers carry recurrent mutations in RNA splicing factor genes, or “spliceosomal mutations,” which induce sequence-specific changes in RNA splicing. In this usage, “cancer” may refer to any dysplastic disease, neoplastic disease, or other disease characterized by disordered cell differentiation, insufficient cell production, impaired cell death, or accelerated cell proliferation. These diseases include solid tumors, malignant ascites, myelodysplastic syndromes, leukemias, lymphomas, and other malignancies and disorders of the bone marrow and hematopoietic system, bone marrow failure syndromes, connective tissue malignancies, metastatic disease, minimal residual disease following transplantation of organs or stem cells, multi-drug resistant cancers, primary or secondary malignancies, angiogenesis related to malignancy, or other forms of cancer. For example, mutations in SRSF2 are primarily found in myeloid malignancies including myelodysplastic syndrome (MDS), acute myeloid leukemia (AML), chronic myelomonocytic leukemia (CMML), and myeloproliferative neoplasms (MPN), as well as solid tumors including uveal melanoma, bladder cancer, lung adenocarcinoma, and others.

The inventors have previously shown that SRSF2 and other common splicing factor mutations cause highly specific changes in RNA splicing mechanisms, such that cancer cells carrying mutations in SRSF2 or other common splicing factor mutations cause highly specific changes in RNA splicing mechanisms, such that cancer cells carrying mutations in SRSF2 or other RNA splicing factors do or do not efficiently remove introns with particular sequences.

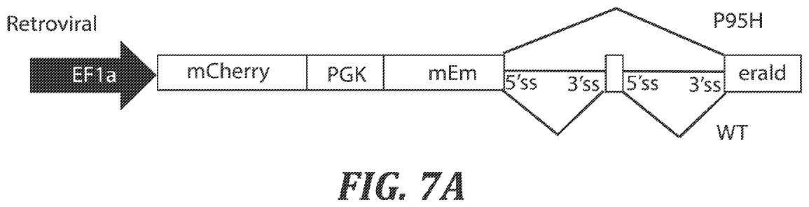

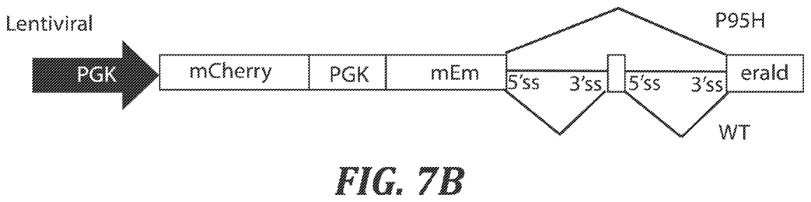

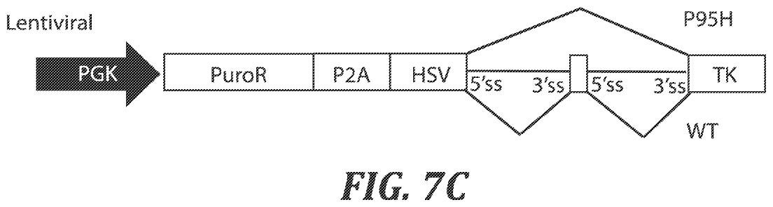

The inventors previously developed a method for constructing synthetic introns that respond to cancer-associated SF3B1 mutations, thereby allowing for specific expression of proteins of interest in SF3B1-mutant cells, but not wildtype cells. Because SF3B1 and SRSF2 mutations cause entirely distinct and mechanistically unrelated changes in RNA splicing, synthetic introns that respond to SF3B1 mutations do not respond to SRSF2 mutations. This was experimentally demonstrated in Example 1 of International Application No. WO 2022/087427 (see, e.g., FIGS. 7A, 7B, and 7F, incorporated herein by reference in its entirety.) Therefore, developing synthetic introns that respond to SRSF2 mutations required an entirely new and distinct effort.

This disclosure describes the generation of a novel approach and related compositions for specific expression of a protein of interest in cells bearing a cancer-associated mutation in SRSF2, but not in cells lacking such a mutation, or vice versa. Several endogenous intronic splicing events have been identified in the human genome that were spliced differently in cancer cells with SRSF2 mutations than in cancer and healthy normal cells without SRSF2 mutations. Alternative splicing events in the following genes: ARFIP2, GTF3C1, MELK, INTS3, and ZNF19 were identified by analysis of human RNA-sequencing data om cancer patients with SRSF2 mutations compared to cancer patients without SRSF2 mutations and to healthy controls. The identified endogenous alternatively spliced events were confirmed via reverse transcriptase polymerase chain reaction (RT-PCR) in human cell lines. Because these endogenous introns are too long to be useful for gene therapy, shorter, synthetic versions of these endogenous introns were created by removing all sequences that were believed to be non-essential for SRSF2 mutation-dependent splicing. Additionally, a completely novel synthetic intron termed MELK/GTF3C1 (356 nt; SEQ ID NO:16), which incorporates a combination of splicing elements utilized in the MELK and GTF3C1 synthetic introns. Shortened synthetic intronic versions were then cloned into the coding sequence (CDS) of a gene of interest in several different vectors to test for functionality.

More specifically, a synthetic intron is described herein that can be inserted into an open reading frame encoding any protein of interest, such that providing the resulting construct into SRSF2-mutant cells results in protein expression, while providing the resulting construct into wild-type (WT) cells results in no protein expression, or vice versa.

Many different cancer types carry recurrent mutations affecting RNA splicing factors. SRSF2 is one of the most commonly mutated splicing factor genes. SRSF2 mutations are particularly common in myelodysplastic syndromes and related disorders, such as chronic myelomonocytic leukemia. SRSF2 mutations preferentially affect the proline residue at position 95 (the P95 residue) and most commonly occur as missense changes, particularly SRSF2P95H/L/R, and cause highly specific changes in RNA splicing regulation. Insertions and deletions in SRSF2 do occur in a recurrent fashion in cancers as well, although less commonly than do missense changes affecting P95, and the inventors have shown that these insertions and deletions preferentially occur near or overlapping with the P95 residue and cause highly similar alternations in RNA splicing regulation (e.g., that all recurrent SRSF2 mutations cause highly specific changes in RNA splicing regulation that are distinct from the splicing dysregulation that results from mutations affecting other RNA splicing factor genes). Therefore, synthetic introns were developed that were spliced differently in cells with or without SRSF2 mutations, in a manner that harnessed the splicing dysregulation cause by SRSF2 mutations.

### Artificial Nucleic Acid Intron Construct

In accordance with the forgoing, in one aspect the disclosure provides an artificial nucleic acid intron construct. The artificial nucleic acid intron construct comprises an intron sequence, hereafter referred to as artificial intron, intron sequence, intron domain, or simply intron. The term “artificial” refers to the sequence of the construct (e.g., including the intron sequence), which does not occur in nature, but has been newly created or derived from a naturally occurring sequence. As used in this context, the term “derived” indicates that the resulting construct sequence has been engineered and contains structural (e.g., sequence) alterations from the naturally occurring sequence.

As explained in more detail in the Examples, the inventors have determined several features that can be leveraged to modify the susceptibility for splicing in cells characterized by a mutation in an RNA splicing factor gene, which permits selective splicing, selective inhibition of splicing, or selective modification of splicing of the intron from the context sequence (e.g., surrounding exonic sequences), compared to cells that lack the mutation in the RNA splicing factor gene.

In some embodiments, synthetic “introns” that respond to SRSF2 mutations frequently comprise a structure having: an (upstream flanking exon)+(upstream intron)+(alternatively spliced “cassette” exon)+(downstream intron)+(downstream flanking exon). In some embodiments, synthetic “introns” that respond to SRSF2 mutations frequently comprise a structure having: an (upstream flanking exon)+(intron)+(downstream flanking exon). In some embodiments, synthetic “introns” that respond to SRSF2 mutations frequently comprise a structure having: an (upstream flanking exon)+(intron containing one or more cryptic 5′ splice sites)+(downstream flanking exon). In some embodiments, synthetic “introns” that respond to SRSF2 mutations frequently comprise a structure having: an (upstream flanking exon)+(intron containing one or more cryptic 3′ splice sites)+(downstream flanking exon). These and other possible structures are illustrated in the figures provided herein. One possible way to capture this is to define a synthetic intron construct as consisting of one of these possibilities:
an alternatively spliced cassette exon flanked by upstream and downstream introns
an intron containing at least two competing 5′ splice sites
an intron containing at least two competing 3′ splice sites
an intron that is sometimes retained (e.g., incompletely spliced).

The term “canonical” 5′ splice site refers to a splice site whose usage results in preservation of the open reading frame if the intron is inserted into a coding DNA sequence and subsequently spliced, such that no in-frame termination codons are introduced into the coding sequence if the canonical 5′ splice site is used during the splicing process. For example, a canonical 5′ splice site may lie at the 5′ end of an intron, such that insertion of this intron into a coding sequence and subsequent usage of the canonical 5′ splice site during splicing results in complete excision of the intron from the mature RNA transcript, thereby preserving the open reading frame. The term “cryptic” 5′ splice site refers to a splice site whose usage results in disruption of the open reading frame if the intron is inserted into a coding DNA sequence and subsequently spliced, such that one or more in-frame termination codons are introduced into the coding sequence if the cryptic 5′ splice site is used during the splicing process. For example, a cryptic 5′ splice site may lie downstream, or 3′ to, the canonical 5′ splice site, such that insertion of this intron into a coding sequence and subsequent usage of the cryptic 5′ splice site during splicing does not result in complete excision of the intron from the mature RNA transcript, thereby disrupting the open reading frame.

Addressing the 5′ splice site, the disclosed artificial intron can comprise any functional canonical 5′ splice site sequence that is typically recognized by splicing factors. Canonical 5′ splice sites are known in the art and are encompassed by the present disclosure. Exemplary, non-limiting canonical 5′ splice sites encompassed by the present disclosure comprise a sequence starting with a GT dinucleotide and can include those selected from GTGAG, GTAAG, GTGCG, GTACG, GTGGG, GTAGG, GTGTG, GTATG, and GTATC. As would be evident to a person of ordinary skill in the art, the canonical 5′ splice site is by definition positioned upstream, or 5′ to, the other recited elements of the intron sequence.

The at least one cryptic 5′ splice site is positioned within about 100 nucleotides (e.g., including within about 90, 80, 70, 60, 50, 40, 30, 20, 10 nucleotides or any range therein) downstream of the canonical 5′ splice site or within about 50 nucleotides (e.g., including within about 40, 30, 20, 10 nucleotides, or any range therein) upstream of the canonical 5′ splice site. As used herein, the term “upstream” refers to a position in a nucleic acid molecule or sequence that is on the 5′ side of the reference position within the nucleic acid molecule or sequence. Conversely, the term “downstream” refers to a position in a nucleic acid molecule or sequence that is on the 3′ side of the reference position within the nucleic acid molecule or sequence.

The artificial intron can comprise a plurality (e.g., 2, 3, 4, 5, 6, 7, 8, 9, 10, or more) of cryptic 5′ splice sites, which can be the same or different from each other. For example, each of the plurality of the cryptic 5′ splice sites can comprise a sequence independently selected from GTA, GTC, GTG, and GTT. In some embodiments, the intron comprises a plurality of cryptic 5′ splice sites within about 100 nucleotides (e.g., including within about 90, 80, 70, 60, 50, 40, 30, 20, 10 nucleotides or any range therein) downstream of the 5′ canonical splice site. In some embodiments, the intron comprises a plurality of cryptic 5′ splice sites within about 100 nucleotides (e.g., including within about 90, 80, 70, 60, 50, 40, 30, 20, 10 nucleotides or any range therein) upstream of the 5′ canonical splice site. In some embodiments, the intron comprises one or more cryptic 5′ splice sites within about 100 nucleotides (e.g., including within about 90, 80, 70, 60, 50, 40, 30, 20, 10 nucleotides or any range therein) downstream of the 5′ canonical splice site and one or more cryptic 5′ splice sites within about 100 nucleotides (e.g., including within about 90, 80, 70, 60, 50, 40, 30, 20, 10 nucleotides or any range therein) upstream of the 5′ canonical splice site.

The term “canonical” 3′ splice site refers to a splice site whose usage results in preservation of the open reading frame if the intron is inserted into a coding DNA sequence and subsequently spliced, such that no in-frame termination codons are introduced into the coding sequence if the canonical 3′ splice site is used during the splicing process. For example, a canonical 3′ splice site may lie at the 3′ end of an intron, such that insertion of this intron into a coding sequence and subsequent usage of the canonical 3′ splice site during splicing results in complete excision of the intron from the mature RNA transcript, thereby preserving the open reading frame. The term “cryptic” 3′ splice site refers to a splice site whose usage results in disruption of the open reading frame if the intron is inserted into a coding DNA sequence and subsequently spliced, such that one or more in-frame termination codons are introduced into the coding sequence if the cryptic 3′ splice site is used during the splicing process. For example, a cryptic 3′ splice site may lie upstream, or 5′ to, the canonical 3′ splice site, such that insertion of this intron into a coding sequence and subsequent usage of the cryptic 3′ splice site during splicing does not result in complete excision of the intron from the mature RNA transcript, thereby disrupting the open reading frame.

Canonical 3′ splice sites are known in the art, which are encompassed by the present disclosure. Exemplary, non-limiting, canonical 3′ splice sites encompassed by the present disclosure comprise a site that ends with an AG dinucleotide and can comprise at least a core sequence of AAG, CAG, GAG, and TAG. The 3′ splice sites can be longer, however, such as selected from the non-limiting list including AACAG, AATAG, ACCAG, ACTAG, ATCAG, ATTAG, AGCAG, AGTAG, CACAG, CATAG, CCCAG, CCTAG, CTCAG, CTTAG, CGCAG, CGTAG, TACAG, TATAG, TCCAG, TCTAG, TTCAG, TTTAG, TGCAG, TGTAG, GACAG, GATAG, GCCAG, GCTAG, GTCAG, GTTAG, GGCAG, and GGTAG, all of which are encompassed by the present disclosure. Exemplary, non-limiting cryptic 3′ splice sites can comprise a sequence selected from AAG, CAG, GAG, TAG, ATG, CTG, GTG, and TTG.

The at least one cryptic 3′ splice site is positioned within about 100 nucleotides (e.g., including within about 90, 80, 70, 60, 50, 40, 30, 20, 10 nucleotides or any range therein) upstream of the canonical 3′ splice site or within about 50 nucleotides (e.g., including within about 40, 30, 20, 10 nucleotides, or any range therein) downstream of the canonical 3′ splice site. As used herein, the term “upstream” refers to a position in a nucleic acid molecule or sequence that is on the 5′ side of the reference position within the nucleic acid molecule or sequence. Conversely, the term “downstream” refers to a position in a nucleic acid molecule or sequence that is on the 3′ side of the reference position within the nucleic acid molecule or sequence.

The artificial intron can comprise a plurality (e.g., 2, 3, 4, 5, 6, 7, 8, 9, 10, or more) of cryptic 3′ splice sites, which can be the same or different from each other. For example, each of the plurality of the cryptic 3′ splice sites can comprise a sequence independently selected from AAG, CAG, GAG, TAG, ATG, CTG, GTG, and TTG. In some embodiments, the intron comprises a plurality of cryptic 3′ splice sites within about 100 nucleotides (e.g., including within about 90, 80, 70, 60, 50, 40, 30, 20, 10 nucleotides or any range therein) upstream of the 3′ canonical splice site. In some embodiments, the intron comprises a plurality of cryptic 3′ splice sites within about 100 nucleotides (e.g., including within about 90, 80, 70, 60, 50, 40, 30, 20, 10 nucleotides or any range therein) downstream of the 3′ canonical splice site. In some embodiments, the intron comprises one or more cryptic 3′ splice sites within about 100 nucleotides (e.g., including within about 90, 80, 70, 60, 50, 40, 30, 20, 10 nucleotides or any range therein) upstream of the 3′ canonical splice site and one or more cryptic 3′ splice sites within about 100 nucleotides (e.g., including within about 90, 80, 70, 60, 50, 40, 30, 20, 10 nucleotides or any range therein) downstream of the 3′ canonical splice site.

A workflow and rules have been established to identify locations for successful insertion of a synthetic intron of the present disclosure into a coding sequence (CDS) encoding a protein of interest. The workflow comprises the steps of:

1. Identifying all dinucleotides (two adjacent nucleotides) within the selected coding sequence (CDS) that are identical to the last and first nucleotides of the upstream and downstream exons flanking the endogenous intron from which the synthetic intron is derived.

2. For each such dinucleotide, computationally inserting the synthetic intron in between the two nucleotides and compute the resulting strengths of the 5′ and 3′ splice sites (e.g., using the MaxEnt algorithm).

3. The positions that are most likely to be successful are those which (a) have splice site strengths which are as close as possible to the strengths of the endogenous spice sites, and (b) divide the CDS into two sequences (exons) of roughly equal length.

4. If steps 2-3 cannot be achieved, then the CDS can be recoded by introducing synonymous codon changes as necessary in order to create the desired splice site strengths.

5. Additional synonymous codon changes can be subsequently introduced to create exonic splicing enhancers (e.g., CCNG, GGNG, CGNG, GCNG, and other sequences that are bound by serine/arginine-rich (SR) proteins or other factors that promote exon recognition) and/or exonic splicing silencers (e.g., TTTGTTCCGT; SEQ ID NO:32, GGGTGGTTTA; SEQ ID NO:33) and other examples of exonic splicing silencers enumerated in Supplemental Table 1 from Wang et al., Cell 119:831-845 (2004), incorporated herein by reference) within the exons in order to alter exon recognition and splicing.

6. The above procedure can be modified to split a CDS into three or more exons and insert two or more different synthetic introns in between the resulting exons, by iteratively applying the procedure in such a manner as to generate exons of appropriate lengths at the end of the procedure.

The above procedure can also be used to insert one or more synthetic introns into an endogenous or exogenous gene, rather than into a CDS as described above (e.g., to insert a synthetic intron into an endogenous gene in the genome). This gene can already contain zero, one, or more introns.

Intron and exon lengths can vary widely in natural settings and still be functionally spliced to result in a contiguous coding sequence in mature RNA transcripts. For example, typical intron lengths in the human genome can be approximately 6,400 nucleotides. Accordingly, the disclosed intron is not limited by length. In some embodiments, the intron is at least about 20 nucleotides, such as 20 nucleotides to about 1500 nucleotides, such as at least about 20 nucleotides to about 1250 nucleotides, about 20 nucleotides to about 1000 nucleotides, about 20 nucleotides to about 900 nucleotides, about 20 nucleotides to about 800 nucleotides, about 20 nucleotides to about 700 nucleotides, about 20 nucleotides to about 600 nucleotides, about 20 nucleotides to about 500 nucleotides, about 100 nucleotides to about 1500 nucleotides, about 100 nucleotides to about 1250 nucleotides, about 100 nucleotides to about 1000 nucleotides, about 100 nucleotides to about 900 nucleotides, about 100 nucleotides to about 800 nucleotides, about 100 nucleotides to about 700 nucleotides, about 100 nucleotides to about 600 nucleotides, about 100 nucleotides to about 500 nucleotides, and any length or range therein.

The intron or exon can be derived from a naturally occurring intron from any eukaryotic organism (referred to as a “source” intron) or from a naturally occurring exon from any eukaryotic organism (referred to as a “source” exon). As indicated above, the term “derived from” refers to the retention of certain structural features of the source intron or exon, but wherein the artificial intron or exon also has certain variations that deviate from the source intron or exon, respectively. In some embodiments, a sequence “derived from” a source can comprise a sequence or subsequence (i.e., subdomain) is about 30%, 35%, 40%, 45%, 50%, 55%, 60%, 65%, 70%, 75%, 80%, 85%, 90%, 95%, 98%, or 99% identical to the source sequence or subsequence (i.e., subdomain), as determined by standard methods. The subdomain can be, e.g., at least about 20, 30, 40, 50, 60, 70, 80, 90, 100, 125, 150, 175, 200, 225, 250, 275, 300, 325, 350, 375, 300, or more contiguous nucleotides of the overall sequence. As a non-limiting example, the intron or exon can be derived from a human wildtype intron or exon, respectively. Examples of such human source introns or exons from which the disclosed intron or exon can be derived include intron 10 (SEQ ID NO:22), exon 10 (SEQ ID NO:23), and intron 11 (SEQ ID NO:24) of the human MELK gene; intron 34 of the human GTF3C1 gene (SEQ ID NO:25); intron 1 of the human ARFIP2 gene (SEQ ID NO:26); intron 4 (SEQ ID NO:27) and exon 5 (SEQ ID NO: 28) of the human INTS3 gene; and exon 3 (SEQ ID NO:29), intron 3 (SEQ ID NO: 30), and exon 4 (SEQ ID NO:31) of the human ZNF19 gene.

The disclosed intron can be obtained, in part, by removing an interior portion from the source intron sequence. Accordingly, in some embodiments, the disclosed intron has a higher sequence similarity to 5′ end and 3′ end domains of the source intron sequence compared to an interior domain of the source sequence. For example, the 5′ end domain and/or 3′ end domain can have a minimal sequence identity to a corresponding 5′ end and/or 3′ end domain of the source intron sequence of at least approximately 25% or 30% and lack any discernable identity or similarity to an interior domain of the source intron sequence. In some embodiments, the disclosed intron has a 5′ end domain with a length of about 10 to about 150 nucleotides (e.g., about 10, 11, 12, 13, 14, 15, 16, 17, 18, 19, 20, 25, 30, 35, 40, 45, 50, 55, 60, 65, 70, 80, 85, 90, 95, 100, 105, 110, 115, 120, 125, 130, 135, 140, 145, or 150 nucleotides), wherein the sequence has at least about 30% sequence identity (e.g., at least about 30%, 40%, 50%, 60%, 70%, 80%, or 90% sequence identity) to a corresponding sequence of the 5′-most 10 to about 150 nucleotides of the wildtype intron. Exemplary wildtype source intron sequences are indicated above. In some embodiments, the disclosed intron has a 3′ end domain with about 50 to about 350 nucleotides (e.g., about 50, 55, 60, 65, 70, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, or 350 nucleotides) having at least 30% sequence identity (e.g., at least about 30%, 40%, 50%, 60%, 70%, 80%, or 90% sequence identity) to a corresponding sequence of the 3′-most 50 to about 350 nucleotides of the wildtype intron. In one embodiment, the disclosed intron has a 5′ end domain with a length of about 15-30 nucleotides (e.g., about 15, 20, 25, or 30 nucleotides) wherein the sequence has at least about 30% sequence identity (e.g., at least about 30%, 40%, 50%, 60%, 70%, 80%, or 90% sequence identity) to a corresponding sequence of a 15-30 nucleotide portion (e.g., the 5′-most 15 to about 30 nucleotides) of the wildtype intron and a 3′ end domain with about 80 to about 130 nucleotides (e.g., about 80, 85, 90, 95, 100, 105, 110, 115, 120, 125, 130 nucleotides) having at least 30% sequence identity (e.g., at least about 30%, 40%, 50%, 60%, 70%, 80%, or 90% sequence identity) to a corresponding sequence of a 80-130 nucleotide portion (e.g., the 3′-most 80 to about 130 nucleotides) of the wildtype intron. In further embodiments, the disclosed intron has a 5′ end domain with a length of about 25 nucleotides (e.g., about 20-30 nucleotides) wherein the sequence has at least about 30% sequence identity (e.g., at least about 30%, 40%, 50%, 60%, 70%, 80%, or 90% sequence identity) to a corresponding sequence of a 25 nucleotide portion (e.g., the 5′-most 20 to about 30 nucleotides) of the wildtype intron and a 3′ end domain with about 80 to about 130 nucleotides (e.g., about 80, 85, 90, 95, 100, 105, 110, 115, 120, 125, 130 nucleotides) having at least 30% sequence identity (e.g., at least about 30%, 40%, 50%, 60%, 70%, 80%, or 90% sequence identity) to a corresponding sequence of 80-130 nucleotides (e.g., the 3′-most 80 to about 130 nucleotides) of the wildtype intron. In some embodiments, the disclosed intron has a 5′ end domain with a length of about 15 nucleotides wherein the sequence has at least about 30% sequence identity (e.g., at least about 30%, 40%, 50%, 60%, 70%, 80%, or 90% sequence identity) to a corresponding sequence of a 15 nucleotide portion (e.g., the 5′-most 15 nucleotides) of the wildtype intron and a 3′ end domain with about 85 nucleotides having at least 30% sequence identity (e.g., at least about 30%, 40%, 50%, 60%, 70%, 80%, or 90% sequence identity) to a corresponding sequence of 85 nucleotides (e.g., the 3′-most 85 nucleotides) of the wildtype intron.

The one or more sequence modifications imposed can be any form of sequence modification, such as insertions, deletions, or substitutions, alone or in any combination. Such modifications can be implemented with any technique available in the art without limitation.

Exemplary embodiments of the one or more sequence modifications are now described. The one or more modifications can comprise one or more of the following in any combination and implemented in any order:
(a) mutating a single nucleotide;
(b) mutating any pair of nucleotides within 10 nucleotides of the 5′ end of the abbreviated intron sequence or 30 nucleotides of the 3′ end of the abbreviated intron sequence;
(c) deleting any consecutive stretch of about 5, 10, 15, 20, 25, 30, 35, 40, 45, 50, 100, 125, 150, 200, 250, or more nucleotides, or any number or range contained therein;
(d) mutating any pair of nucleotides within the 5 nucleotides upstream of and 5 nucleotides downstream of each SSNG sequence, where S=C or G and N=any nucleotide;
(e) deleting any combination of SSNG sequences;
(f) inserting any combination of SSNG sequences;
(g) mutating any one or more branchpoint and flanking sequence contexts to one or more strong branchpoint and flanking sequence contexts.
(h) mutating any four consecutive nucleotides to cAGg;
(i) inserting a polypyrimidine tract immediately followed by a 3′ splice site at any position;
(j) mutating any consecutive stretch of nucleotides to one or more thymines;
(k) mutating all pyrimidines within any six or more consecutive positions to guanines;
(l) inserting a strong branchpoint and flanking sequence context at any position;
(m) inserting one or more intronic splicing enhancers, including SSNG sequences, at any position; and
(n) inserting one or more intronic splicing silencers at any position.

In some embodiments, any 2, 3, 4, 5, 6, 7, 8, 9, 10, 11, 12, 13, or all the modifications (a) through (n) are implemented. Terms such as “upstream”, “downstream”, are described in more detail above in the context of the artificial nucleic acid intron construct. Such descriptions apply here and are not repeated for brevity.

In some embodiments, the polypyrimidine tract immediately followed by a 3′ splice site, as described in modification (i), comprises at least six consecutive nucleotides containing at least four pyrimidines. The stretch of the at least four pyrimidines are immediately followed by a sequence selected from AAG, CAG, GAG, TAG, ATG, CTG, GTG, TTG, or any other known 3′ splice site.

In some embodiment, a sequence serving as a splice enhancer can be incorporated. Sequences serving as splicing enhancers or splicing silencers are described in more detail above and are encompassed by this aspect of the disclosure. In some embodiments, the one or more exonic splicing enhancers is/are selected from CCNG, CGNG, GCNG, GGNG, and other sequences with known enhanced likelihood of binding by serine/arginine-rich (SR) proteins, in any combination. Sequences serving as splicing enhancers are described in more detail above and are encompassed by this aspect of the disclosure. In some embodiments, the one or more exonic splicing enhancers is/are selected from CCNG, CGNG, GCNG, GGNG, and other sequences with known enhanced likelihood of binding by serine/arginine-rich (SR) proteins, in any combination. The designation of N refers to any nucleotide.

In some embodiments, the artificial nucleic acid intron construct is selected from SEQ ID NOS: 9-18 or has an intron comprising a sequence with at least 70% sequence identity (e.g., about 70%, 75%, 80%, 85%, 90%, 95% or 98% sequence identity) of a sequence selected from SEQ ID NOS: 9-18. In some embodiments, the sequence identity of the disclosed synthetic intron to the reference SEQ ID NOS: 9-18 and is higher at the 5′ end and/or the 3′ end. For example, in some embodiments, the disclosed intron has a 5′ end subsequence with at least 70% sequence identity (e.g., about 70%, 75%, 80%, 85%, 90%, 95% or 98% sequence identity) to the 5′-most 15 nucleotide positions of one of SEQ ID NOS: 1-18. In some embodiments, the disclosed intron has a 3′ end subsequence with at least 70% sequence identity (e.g., about 70%, 75%, 80%, 85%, 90%, 95% or 98% sequence identity) to the 3′-most 50 nucleotide positions of one of SEQ ID NOS: 1-18.

The disclosed synthetic introns fall into one of five (5) families:
Family 1: synMELK derived from intron 10, exon 10, and intron 11 of the human MELK gene;
Family 2: synGTF3C1 derived from intron 34 of the human GTF3C1 gene;
Family 3: synARFIP2 derived from intron 1 of the human ARFIP2 gene;
Family 4: synINTS3 derived from intron 4 or from intron 4 and exon 5 of the human INTS3 gene; and
Family 5: synZNF19 derived from exon 3 and intron 3 of the human ZNF19 gene.

Each of the SRSF2 mutation specific synthetic introns indicated above are, in exemplary wild-type contexts, flanked by upstream and downstream sequences as follows:

ParentSynMELK: in total is 623 nt. The first 50 nt of MELK intron 10 which encompasses the endogenous 5′ splice site (ss) of intron 10 was ligated to the last 200 nt of MELK intron 10 which encompasses the endogenous 3′ ss of intron 10. This sequence was ligated to endogenous MELK exon 10 (123 nt). This segment was ligated to the first 50 nt of MELK intron 11 which encompasses the endogenous 5′ ss of intron 11 and was ligated to the last 200 nt of MELK intron 11 which encompasses the endogenous 3′ ss of intron 11. Additional shortened variations of this parent synthetic intron have been created and are described infra.

ParentSynGTF3C1: in total is 325 nt. The first 125 nt of GTF3C1 intron 34 which encompasses the alternative 5′ ss (1-75 nt) and the canonical 5′ ss (76-125 nt). This sequence was ligated to the last 200 nt of GTF3C1 intron 34 which encompasses the endogenous 3′ splice site of intron 34. Additional shortened variations of this parent synthetic intron have been created and are described infra.

ParentSynARFIP2: in total is 250 nt. The first 50 nt of ARFIP2 intron 1 encompasses the endogenous 5′ ss of intron 1 and was ligated to the last 200 nt of ARFIP2 intron 1 which comprises the endogenous canonical 3′ ss (160-200 nt). Additional shortened variations of this parent synthetic intron have been created and are described infra.

ParentSynINTS3: in total is 411 nt. All of INTS3 exon 4 (114 nt) was ligated to 174 nt of INTS3 intron 4. Inserted at position 175 of the synthetic intron was the sequence GCCGCCA which encompasses a Kozak sequence (GCCGCC) and an adenosine nucleotide. This sequence is followed by the remaining 33 nt of INTS3 intron 4, 83 nt of INTS3 exon 5 and full-length HSV-TK minus the first 3 nucleotides that code for methionine. The addition of a Kozak sequence functions as a protein translation initiation site and the addition of adenosine allows for a methionine residue to be translated immediately after the Kozak sequence. This allows for the synthesis of a transcript with the first 117 nt (coding for 39 amino acid residues) derived from INTS3 intron 4 and exon 5 followed by the entire HSV-TK coding sequence except for the first 3 nucleotides coding for methionine.

ParentSynZNF19: in total 378 nt. All of ZNF19 exon 3 (30 nt) was ligated to the first 50 nt of ZNF3 intron 3 which encompasses the endogenous 5′ ss. This segment was ligated to the last 200 nt of ZNF19 intron 3 encompassing the canonical 3′ ss. This segment was then added to the first 55 nt of the ZNF19 alternatively ss. The nucleotides gccatg were added to this sequence to create a Kozak sequence for protein translation initiation followed by 3 nucleotides to code for a methionine residue. The remaining 37 nt of the ZNF19 alternative splice sequence was then added followed by the coding sequence of HSV-TK with the exception of the first adenosine. This allows for the synthesis of a transcript with the first 439 nt (coding for 13 amino acids) derived from the ZNF19 3′ alternative splice sequence followed by the entire HSV-TK coding sequence whereby the first amino acid coded for is valine instead of methionine.

In a further embodiment, the MELK-derived synthetic intron comprising in total 623 nucleotides (nt). The first 250 nt are derived from MELK intron 12, a 123 nt alternatively spliced (“cassette”) exon that is derived from exon 12 of the MELK gene, and a 250 nt intron that is derived from intron 13 of the endogenous MELK gene.

In a further embodiment, the derived introns comprise a canonical 5′ splice site comprising a GT dinucleotide immediately followed by a consensus 5′ splice site context. Exemplary 5′ splice site contexts include, but are not limited to AAG, GAG, and GTG, which would result in a sequence of GTAAG, GTGAG, GTGTG, respectively, when including the GT dinucleotide.

In a further embodiment, the derived introns comprise at least one cryptic 5′ splice site located at least 5 nucleotides upstream of the canonical 5′ splice site. The at least one cryptic 5′ splice site comprises a GT dinucleotide and has a sequence that is a weaker 5′ splice site than is the canonical 5′ splice site. The relative strength or weakness can be estimated computationally, for example with the MaxEntScan algorithm or similar methods.

In a further embodiment, the canonical 3′ splice site of the derived introns comprise an AG dinucleotide immediately preceded by a C or T, which would result in a sequence of CAG or TAG, respectively.

In a further embodiment, the derived introns comprise at least one cryptic 3′ splice site located at least 5 nucleotides upstream of the canonical 3′ splice site. The at least one cryptic 3′ splice site comprises an AG dinucleotide and has a sequence that is a weaker 3′ splice site than is the canonical 3′ splice site. The relative strength or weakness can be estimated computationally, for example with the MaxEntScan algorithm or similar methods.

The embodiments of the intron, including those described above, are configured to be spliced differently in a cell (e.g., cancer cell) comprising a change-of-function or loss-of-function mutation in a recurrently mutated RNA splicing factor gene. The difference in splicing is relative to the splicing pattern of the intron in a cell lacking a change-of-function or loss-of-function mutation in a recurrently mutated RNA splicing factor gene. In some embodiments, the artificial intron is more likely to be recognized and spliced in a cell comprising a change-of-function or loss-of-function mutation in a recurrently mutated RNA splicing factor gene compared to in a cell without the mutation. In some embodiments, the artificial intron is less likely to be recognized and spliced in a cell comprising a change-of-function or loss-of-function mutation in a recurrently mutated RNA splicing factor gene compared to in a cell without the mutation. In some embodiments, the artificial intron is preferentially partially spliced out in a cell comprising a change-of-function or loss-of-function mutation in a recurrently mutated RNA splicing factor gene, such that a portion of the intron is not excised from the mature transcript, while the entire intron is preferentially spliced out in a cell without the mutation. In some embodiments, the entire intron is preferentially spliced out in a cell comprising a change-of-function or loss-of-function mutation in a recurrently mutated RNA splicing factor gene, while the intron is partially spliced out in a cell without the mutation, such that a portion of the intron is not excised from the mature transcript.

There are a variety of RNA splicing factor genes that have recurrent mutations. As used herein, the term “recurrent” refers to a mutation that has been observed in multiple cell types (e.g., multiple cancer types) and/or in multiple individuals with the same cancer type, such that there is an established association with the recurrent mutation and the aberrant phenotype of the cell (e.g., cancer phenotype).

To illustrate, an exemplary and non-limiting RNA splicing factor gene encompassed by this disclosure is SRSF2, which can have a recurrent mutation that leads to a change-of-function or loss-of-function in the expressed splicing factor. Various recurrent mutations in SRSF2 have been previously characterized and are encompassed by this disclosure.

The artificial nucleic acid intron construct can consist of the intron sequence, consist essentially of the intron sequence, or comprise the intron sequence with additional domains or elements. For example, in some embodiments, the artificial nucleic acid intron construct comprises the artificial intron, such as described above, in addition to coding sequence flanking one or both ends. In one embodiment, the artificial nucleic acid intron construct further comprises a first exon domain and a second exon domain, wherein the intron is disposed between the first exon domain and the second exon domain. Upon proper splicing, the combination of the first exon domain and the second exon domain in a contiguous sequence, i.e., without the intron) encodes part or all of a protein of interest. In an alternative embodiment the artificial intron comprises at least one alternatively spliced cassette exon is embedded within the artificial intron. In some embodiments, the artificial nucleic acid intron construct can comprise, or be comprised of, an expression cassette to facilitate transcription. An expression cassette in the present context is a construct that generally includes a gene (e.g., including coding and noncoding, or intron, sequence) and regulatory non-coding sequence to facilitate expression. In some embodiments, the expression cassette comprises a promoter sequence and the gene sequence. In additional embodiments, the expression cassette can further comprise a 5′ untranslated region and/or a 3′ untranslated region.

The term “promoter” refers to a regulatory nucleotide sequence that can activate transcription (expression) of a gene. As indicated, a promoter is typically located upstream of a gene, but can be located at other regions proximal to the gene, or even within the gene. The promoter typically contains binding sites for RNA polymerase and one or more transcription factors, which participate in the assembly of the transcriptional complex. As used herein, the term “operatively linked” indicates that the promoter and the gene region (e.g., including coding and noncoding, or intron, sequence) are configured and positioned relative to each other a manner such that the promoter can activate transcription of the encoding nucleic acid by the transcriptional machinery of the cell. The promoter can be constitutive or inducible. Constitutive promoters can be determined based on the character of the target cell and the particular transcription factors available in the cytosol. A person of ordinary skill in the art can select an appropriate promoter based on the intended use, as various promoters are known and commonly used in the art. In some embodiments, the nucleic acid intron construct comprises an expression cassette comprising the first exon domain, the intron, the second exon domain, and a promoter sequence operatively linked thereto.

The expression cassette can be incorporated into a vector, such as a plasmid or viral vector, configured for delivery into a cell. Accordingly, in some embodiments, the disclosure provides a vector comprising the artificial nucleic acid intron construct described above. The vector can be any construct that facilitates the delivery of the nucleic acid to the target cell and/or expression of the nucleic acid within the cell. The vectors can be viral vectors, circular nucleic acid constructs (e.g., plasmids), or nanoparticles. Various viral vectors are known in the art and are encompassed by the present disclosure. See, e.g., Machida, C. A. (ed.), Viral Vectors for Gene Therapy: Methods and Protocols, Humana Press, Totowa, New Jersey (2003); Muzyczka, N., (ed.), Current Topics in Microbiology and Immunology: Viral Expression Vectors, Springer-Verlag, Berlin, Germany (2012), each incorporated herein by reference in its entirety. In some embodiments, the viral vector is an adeno-associated virus (AAV) vector, an adenovirus vector, a herpes simplex virus vector, a retrovirus vector, a lentivirus vector, an alphavirus vector, a flavivirus vector, a rhabdovirus vector, a measles virus vector, a Newcastle disease virus vector, a Coxsackievirus vector, or a poxvirus vector. An exemplary embodiment of an AAV vector includes the AAV2/5 serotype.

In another aspect, the disclosure provides a method of modifying a nucleic acid sequence to permit selective modification of expression in a cell characterized by a mutation in an RNA splicing factor gene. The selective modification of expression can refer to selective expression in the cell, e.g., increased expression in the cell, compared to a cell without the mutation. Increased expression can include any expression in the cell if the reference cell without the expression has no detectable expression. For example, the cell can be a cancer cell with a recurrently mutated RNA splicing factor and the nucleic acid is modified to be selectively expressed to produce a protein in the cancer cell, but to avoid having the production of the protein in non-cancer cells. Alternatively, the selective modification of expression can refer to selective reduction or lack of expression in the cell, compared to a cell without the mutation. For example, the cell can be a cancer cell with a recurrently mutated RNA splicing factor and the nucleic acid is modified to be selectively expressed to produce a protein in the non-cancer cells, but to avoid having the production of the protein in the cancer cells. Furthermore, in the present context, the term “expressed” and grammatical variants thereof refer to successful transcription, processing (including splicing) to produce a mature transcript (i.e., mRNA), and translation of the mature transcript to produce a functional polypeptide molecule (i.e., protein). The artificial nucleic acid introns disclosed herein can modify the expression, i.e., the ultimate production of protein, by being selectively subject to different patterns of splicing (i.e., being selectively susceptible or resistant to excision of the full intron versus excision of none or only part of the intron) from the initial transcribed RNA (i.e., pre-mRNA) before translation occurs.

The workflow for identifying successful locations for inserting a synthetic intron into the coding sequence (CDS) encoding a protein of interest is as follows:

1. Identify all dinucleotides (two adjacent nucleotides) within the CDS that are identical to the last and first nucleotides of the upstream and downstream exons flanking the endogenous intron from which the synthetic intron is derived.

2. For each such dinucleotide, computationally insert the synthetic intron in between the two nucleotides and compute the resulting strengths of the 5′ and 3′ splice sites (e.g., using the MaxEnt algorithm).

3. The positions that are most likely to be successful are those which (a) have splice site strengths which are as close as possible to the strengths of the endogenous splice sites, and (b) divide the CDS into two sequences (exons) of roughly equal length.

4. If steps 2-3 cannot be achieved, then the CDS can be re-coded by introducing synonymous codon changes as necessary in order to create the desired splice site strengths.

5. Additional synonymous codon changes can be subsequently introduced to create exonic splicing enhancers (e.g., CCNG, GGNG, CGNG, GCNG, and other sequences that are bound by serine/arginine-rich (SR) proteins or other factors that promote exon recognition) and/or exonic splicing silencers (e.g., TTTGTTCCGT (SEQ ID NO:32), GGGTGGTTTA (SEQ ID NO:33)) and other examples exonic splicing silencers enumerated in Supplemental Table S1 from Wang et al., Cell 119:831-845 (2004); incorporated herein by reference) within the exons in order to alter exon recognition and splicing.

6. The above procedure can be modified to split a CDS into three or more exons, and insert two or more different synthetic introns in between the resulting exons, by iteratively applying the procedure in such a manner as to generate exons of appropriate lengths at the end of the procedure.

In some embodiments, the selecting activity in step (3) further comprises the following design steps:
(a) computationally inserting the sequence of the artificial nucleic acid intron at the selected insertion point to create a hypothetical exonic flanking sequence context for a 5′-most 5′ splice site and a 3′-most 3′ splice site;
(b) computing strength scores for the 5′-most 5′ splice site and the 3′-most 3′ splice site, respectively, in their hypothetical exonic contexts;
(c) comparing the computed strength scores for the 5′ splice site and 3′-most 3′ splice site within their hypothetical exonic contexts to strength scores of the respective 5′-most 5′ splice site and 3′-most 3′ splice site of the wildtype intron in its wildtype exonic context from which the artificial nucleic acid intron is derived; and
(d) selecting a dinucleotide wherein computational insertion of the artificial nucleic acid intron sequence results in strength scores for the 5′-most 5′ splice site and 3′-most 3′ splice site in their hypothetical exonic contexts that differ by about 50% or less of the respective 5′ splice site and 3′-most 3′ splice site scores of the wildtype intron in its wildtype exonic context (i.e., the scores in the hypothetical exonic contexts are between 50% and 150% of the respective scores in the wildtype exonic context).

Strength scores can be computed using any available program or algorithm that models splicing performance. For example, in some non-limiting embodiments, the strength scores can be computed with a standard method such as MaxEntScan::scores5ss, MaxEntScan::score3ss, HumanSplicingFinder, and other similar algorithms known in the art. See, e.g., Desmet, et al., Human Splicing Finder: an online bioinformatics tool to predict splicing signals, Nucleic Acids Res. 2009 May; 37 (9): e67; and Yeo, G. and Burge C., Maximum entropy modeling of short sequence motifs with applications to RNA splicing signals, J Comput Biol. 2004; 11 (2-3): 377-94, each of which is incorporated herein by reference in its entirety.

In some embodiments, the selecting activity in step (3) can further comprise introducing one or more synonymous codon mutations into the nucleic acid that improve or weaken one or both scores for the 5′-most 5′ and 3′-most 3′ splice sites in their hypothetical exonic contexts. Synonymous codon mutations are substitutions in the encoding DNA sequence that encode for the same amino acid (i.e., are redundant to) the original sequence. By this approach, the relative splice strength scores can be adjusted as necessary for the desired application of the artificial intron construct.

In some embodiments, the method can further comprise introducing one or more synonymous codon mutations into the nucleic acid that result in creation of one or more exonic splicing enhancers and/or one or more exonic splicing silencers. As indicated above, exonic enhancers and/or silencers can be incorporated to fine tune the construct's susceptibility to splicing. A practitioner might include an exonic splicing enhancer if the artificial intron construct is not effectively spliced out at high rates even in target cells, e.g., cells with recurrent mutation in an RNA splicing factor gene. Conversely, a practitioner might incorporate an exonic splicing silencer if the artificial intron is spliced out at high rates in the target cells, including at unacceptable rates in wildtype cells without the RNA splicing factor gene mutation (e.g., wild type reference cells). Sequences serving as splicing enhancers or splicing silencers are described in more detail above and are encompassed by this aspect of the disclosure. In some embodiments, the one or more exonic splicing enhancers is/are selected from CCNG, CGNG, GCNG, GGNG, and other sequences with known enhanced likelihood of binding by serine/arginine-rich (SR) proteins, in any combination. The designation of N refers to any nucleotide. In some embodiments, the one or more exonic splicing silencers is/are selected from TTTGTTCCGT (SEQ ID NO:32), GGGTGGTTTA (SEQ ID NO:33), GTAGGTAGGT (SEQ ID NO:34), TTCGTTCTGC (SEQ ID NO:35), GGTAAGTAGG (SEQ ID NO:36), GGTTAGTTTA (SEQ ID NO:37), TTCGTAGGTA (SEQ ID NO:38), GGTCCACTAG (SEQ ID NO:39), TTCTGTTCCT (SEQ ID NO:40), TCGTTCCTTA (SEQ ID NO:41), GGGATGGGGT (SEQ ID NO:42), GTTTGGGGGT (SEQ ID NO:43), TATAGGGGGG (SEQ ID NO:44), GGGGTTGGGA (SEQ ID NO:45), TTTCCTGATG (SEQ ID NO:46), TGTTTAGTTA (SEQ ID NO:47), TTCTTAGTTA (SEQ ID NO:48), GTAGGTTTG, GTTAGGTATA (SEQ ID NO:49), TAATAGTTTA (SEQ ID NO:50), TTCGTTTGGG (SEQ ID NO:51), and the like, or sequences with at least 50% identity thereto. See, e.g., Wang, Z., et al., Systematic Identification and Analysis of Exonic Splicing Silencers, Cell 119 (6): 831-845, 2004, incorporated herein by reference in its entirety, for disclosure of exonic splicing silencers encompassed by the present disclosure.

In some embodiments, the disclosed steps are performed multiple times for a given target nucleic acid molecule such that two or more (e.g., 3, 4, 5, 6, or more) artificial intron molecules are ultimately inserted into the target nucleic acid molecule. The insertion of the two or more artificial introns results in a plurality of target molecule domains, wherein each of the plurality of target molecule domains are separated by the artificial intron molecules. The plurality of target molecule domains can each correspond to a different portion of the same CDS. The plurality of separated target molecule domains can be of any size in relation to each other. In some embodiments, however, each of the plurality of the separated target molecule domains is at least about 50% (e.g., about 50%, 55%, 60%, 65%, 70%, 75%, 80%, 85%, 90%, 95%) of the length of the longest separated target molecule domain.

The target nucleic acid molecule can an isolated nucleic acid molecule with a protein-coding sequence (CDS) that encodes a protein of interest. The target nucleic acid modified with the artificial intron construct molecule is configured to permit selective modified expression (e.g., selective increased expression, or alternately selective lack of expression) of the protein of interest in a cell characterized by a mutation in an RNA splicing factor gene. As indicated above, the term selective refers to the modified expression (e.g., increased or lack of expression) in the cell characterized by a mutation in an RNA splicing factor gene in contrast to reference cells characterized by the wildtype RNA splicing factor gene. As described above, the term expression refers to the ultimate production of a protein product translated from a gene transcript. The expression involves proper splicing of the intron construct to permit expression of the final protein product. The artificial intron construct can be configured for selective proper splicing by the cell in the context of the mutated RNA splicing factor, or alternatively to selectively prevent proper splicing by the cell in the context of the mutated RNA splicing factor.

In some embodiments, the method further comprises introducing the modified target nucleic acid molecule to a cancer cell with a mutation in an RNA splicing factor gene and permitting expression, or alternately selective lack of expression, of the protein of interest. The modified target nucleic acid molecule can be incorporated into a functional expression cassette, as described above. In some embodiments, the modified target nucleic acid molecule is incorporated into an expression vector, such as a viral expression vector, or other cell delivery/expression system, as described herein, to promote delivery into and expression in the cancer cell.

In some embodiments, the target nucleic acid molecule is a gene in the chromosome of a cell, wherein the gene encodes a protein of interest, and the modified target nucleic acid molecule is configured for selective expression, or alternately selective lack of expression, in a cell characterized by a mutation in an RNA splicing factor gene.

In some embodiments, the cell is a cancer cell and the mutation in an RNA splicing factor gene is a change-of-function or loss-of-function mutation in a recurrently mutated RNA splicing factor gene. As indicated above, the artificial intron sequence is configured to be spliced differently in a cancer cell comprising the change-of-function or loss-of-function mutation in the recurrently mutated RNA splicing factor gene, relative to the splicing pattern of the intron in a cell lacking the change-of-function or loss-of-function mutation in the recurrently mutated RNA splicing factor gene. The different splicing pattern of the artificial intron sequence results in production of different mature transcripts of the modified target nucleic acid molecule in a cancer cell comprising the change-of-function or loss-of-function mutation in the recurrently mutated RNA splicing factor gene, relative to the splicing pattern of the intron in a cell lacking the change-of-function or loss-of-function mutation in the recurrently mutated RNA splicing factor gene. The production of different mature transcripts of the modified nucleic acid molecule permits either selective expression, or alternately selective lack of expression, of a desired protein from the target nucleic acid molecule in the cancer cell, and the opposite pattern in a cell lacking the change-of-function or loss-of-function mutation in the recurrently mutated RNA splicing factor gene.

RNA splicing factor genes that are subject to recurrent mutations are known. As used herein, the term “recurrent mutation” and grammatical variants thereof refer to the mutation (or mutations) being observed in multiple individuals such that there is an association between the mutation and the altered functionality of the RNA splicing factor expressed from the mutated gene. In some embodiments, the mutation (or mutations) is associated with or demonstrably contribute to the phenotype of a transformed (e.g., cancer) cell. One illustrative, non-limiting example of the RNA splicing factor gene is SRSF2, which is known to have recurrent mutations associated with change of function.

In another aspect, the disclosure provides a method of selectively expressing, or alternately selectively not expressing, a gene of interest in a cell. The cell comprises a change-of-function or loss-of-function mutation in a recurrently mutated RNA splicing factor gene.

The method comprises introducing to the cell an expression cassette comprising a coding sequence (CDS) interrupted by at least one artificial nucleic acid intron described hereinabove. The expression cassette further comprises a promoter operatively linked to the CDS. The terms “promoter” and “operatively linked” are defined above. The method further comprises permitting transcription of the coding sequence and modified splicing of the transcript induced by the artificial nucleic acid intron in the resulting transcript in conjunction with the mutated splicing factor. As described above, the modified splicing of the transcript can encompass an increased likelihood of a splicing event such that the resulting protein is expressed, or a decreased likelihood of a splicing event such that the resulting translation product is not the protein in its functional form. The modification is selective in that the outcome is specific to the cell(s) with the cell comprising a change-of-function or loss-of-function mutation in a recurrently mutated RNA splicing factor gene in comparison to a cell without the change-of-function or loss-of-function mutation. It will be appreciated that while the RNA splicing factor expressed from the mutated RNA splicing factor gene is necessary for the modified splicing of the artificial intron, it does not itself perform the direct catalytic reaction of splicing. Instead, the mutated splicing factor alters splice site, intron, or exon recognition to allow subsequent splicing of the artificial intron domain by other factors. Alternately, for the case of recurrent loss-of-function mutations in an RNA splicing factor gene, absence of the functional RNA splicing factor results in modified recognition or loss of recognition of splice sites, introns, or exons.

The expression cassette can be incorporated into an expression vector, such as a viral expression vector, or other cell delivery/expression system, as described herein, to promote delivery into and expression in the cell.

The cell can be a cancer cell and the mutation in an RNA splicing factor gene can be a change-of-function or loss-of-function mutation in a recurrently mutated RNA splicing factor gene, as described above. As described above, an exemplary and non-limiting RNA splicing factor gene encompassed by the disclosure is SRSF2.

The cancer cell can be from any cancer, myelodysplastic syndrome or other hematologic disease, or other dysplastic, proliferative, or malignant disease that is characterized by or associated with a recurrently mutated RNA splicing factor gene. For example, with respect to recurrent mutations in SRSF2, the cancer is a myelodysplastic syndrome (MDS), chronic myelomonocytic leukemia (CMML), acute myeloid leukemia (AML), myeloproliferative neoplasms (MDN), uveal melanoma, bladder cancer, lung adenocarcinoma, or other neoplasm with recurrent SRSF2 mutations.

In some embodiments, upon splicing of the at least one artificial nucleic acid intron from the gene transcript, the mature transcript, i.e., with the CDS lacking the intron, encodes a functional therapeutic protein. The functional therapeutic protein can be any protein that, when expressed, can have a detrimental effect on the cancer cell, whether directly or indirectly, alone or in conjunction with other therapeutics or immune system factors. For example, the functional therapeutic protein can be a toxin, chemokine, cytokine, growth factor, targetable cell-surface protein, targetable antigen, druggable enzyme, detectable marker, and the like. Exemplary functional therapeutic proteins are described in more detail below.

In another aspect, the disclosure provides a method of treatment in a subject for a subject with cancer. The method incorporates cancer-specific gene therapy. In this aspect, the cancer is characterized by a change-of-function or loss-of-function mutation in a recurrently mutated RNA splicing factor gene, as have been described above. The method comprises administering to the subject an effective amount of a therapeutic composition comprising an expression cassette comprising a coding sequence (CDS) interrupted by at least one artificial nucleic acid intron as described herein. The expression cassette further comprises a promoter operatively linked to the CDS, as described herein.

As described above, an exemplary and non-limiting RNA splicing factor gene encompassed by the disclosure that can have recurrent mutations is SRSF2.

The cell can be from any cancer, myelodysplastic syndrome or other hematologic disease, or other dysplastic, proliferative, or malignant disease that is characterized by or associated with a recurrently mutated RNA splicing factor gene. For example, with respect to recurrent mutations in SRSF2, the cancer is a myelodysplastic syndrome (MDS), chronic myelomonocytic leukemia (CMML), acute myeloid leukemia (AML), myeloproliferative neoplasms (MDN), uveal melanoma, bladder cancer, lung adenocarcinoma, or other neoplasm with recurrent SRSF2 mutations.

In some embodiments, upon splicing of the at least one artificial nucleic acid intron from the gene transcript, the mature transcript, i.e., with the CDS lacking the intron, encodes a functional therapeutic protein. The functional therapeutic protein can be any protein that, when expressed, can have a detrimental effect on the cancer cell, whether directly or indirectly, alone or in conjunction with other therapeutics or immune system factors. For example, the functional therapeutic protein can be a toxin, chemokine, cytokine, growth factor, targetable cell-surface protein, targetable antigen, druggable enzyme, detectable marker, and the like. Exemplary functional therapeutic proteins are now described.

In some embodiments, the functional therapeutic protein is a chemokine, cytokine, or growth factor, and wherein the chemokine, cytokine, or growth factor stimulates an increased immune response against the cancer cell. As non-limiting examples, the functional therapeutic protein can be IFNα, IFNβ, IFNγ, IL-2, IL-12, IL-15, IL-18, IL-24, TNFα, GM-CSF, and the like, or functional domains or derivatives thereof. Exemplary cytokines and derivatives are known (see, e.g., Levin, A. M., et al. Exploiting a natural conformational switch to engineer an interleukin-2 ‘superkine’. Nature 484 (7395): 529-533 (2012) and Silva, D. A., et al. De novo design of potent and selective mimics of IL-2 and IL-15. Nature 565 (7738): 186-191 (2019), incorporated herein by reference in its entirety) and are encompassed by the disclosure. In one embodiment, the functional therapeutic protein is IL-2 or IL-2-derived variant proteins, such as IL-2 “superkines,” that exhibit desirable therapeutic properties such as enhanced activation of cytotoxic CD8⁺ T cells. For example, as described in Example 2 below, a CDS for IL-2 was divided by the disclosed artificial introns in an expression cassette. When transcribed and properly spliced in leukemic and melanoma cells with a change of function mutation in the RNA splicing factor gene SRSF2, the exons are combined in the mRNA leading to proper expression and secretion of the IL-2 protein by the cells. Exemplary IL-2 exon sequences that can be used in conjunction with the disclosed artificial introns to implement such cell-specific expression of functional IL-2 protein include exon 1 and exon 2 of IL-2. See Example 1 infra. Use of these disclosed exons, or exons with sequences that encode the same protein sequences in an expression cassette with the disclosed artificial intron constructs, and functional variants and derivatives thereof, are encompassed by the disclosure.

In some embodiments, the functional therapeutic protein is a targetable cell-surface protein or targetable antigen. In further embodiments, the method further comprises administering to the subject an effective amount of a second therapeutic composition comprising an affinity reagent that specifically binds the target cell-surface protein or targetable antigen. Useful targetable antigens include proteins that are not typically expressed in healthy cells, or not typically expressed at high levels in healthy cells, such that a targeting affinity reagent will bind with substantial specificity to the transformed cell induced to express the targetable antigen. Non-limiting examples of targetable cell-surface proteins or targetable antigens include CD19, CD22, CD23, CD123, ROR1, truncated EGFR (EGFRt), or functional domains thereof, and the like.

As used herein, the term “affinity reagent” refers to a molecule that specifically binds to a target antigen, and typically a specific epitope on a target antigen. As used herein, the term “specifically bind” or variations thereof refer to the ability of the affinity reagent(s) to bind to the antigen of interest (e.g., the targetable antigen or cell-surface protein), without significant binding to other molecules, under standard conditions known in the art.

Exemplary, non-limiting categories of affinity reagent include antibodies, an antibody-like molecule (including antigen-binding fragments of antibodies and derivatives thereof), peptides that specifically interact with a particular antigen (e.g., peptibodies), antigen-binding scaffolds (e.g., DARPins, HEAT repeat proteins, ARM repeat proteins, tetratricopeptide repeat proteins, and other scaffolds based on naturally occurring repeat proteins, and the like (See, e.g., Boersma and Plückthun, Curr. Opin. Biotechnol. 22:849-857 (2011), and references cited therein, each incorporated herein by reference in its entirety)), aptamers, or a functional antigen-binding domain or fragment thereof.

In some embodiments, the indicated affinity reagent is an antibody. As used herein, the term “antibody” encompasses antibodies and antigen-binding antibody fragments or derivatives thereof, derived from any antibody-producing mammal (e.g., mouse, rat, rabbit, camel, and primate including human), that specifically bind to an antigen of interest (e.g., the targetable antigen or cell-surface protein). Exemplary antibodies include multi-specific antibodies (e.g., bispecific antibodies); humanized antibodies; murine antibodies; chimeric, mouse-human, mouse-primate, primate-human monoclonal antibodies; and anti-idiotype antibodies. The antigen-binding molecule can be any intact antibody molecule or fragment or derivative thereof (e.g., with a functional antigen-binding domain).

An antibody fragment is a portion derived from or related to a full-length antibody, preferably including the complementarity-determining regions (CDRs), antigen-binding regions, or variable regions thereof. Illustrative examples of antibody fragments and derivatives useful in the present disclosure include Fab, Fab′, F(ab)₂, F(ab′)₂ and Fv fragments, nanobodies (e.g., V_{H}H fragments and V_{NAR} fragments), linear antibodies, single-chain antibody molecules, multi-specific antibodies formed from antibody fragments, and the like. Single-chain antibodies include single-chain variable fragments (scFv) and single-chain Fab fragments (scFab). A “single-chain Fv” or “scFv” antibody fragment, for example, comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Single-chain antibodies can also include diabodies, triabodies, and the like. Antibody fragments can be produced recombinantly, or through enzymatic digestion.

The above affinity reagents do not have to be naturally occurring or naturally derived, but can be further modified to, e.g., reduce the size of the domain or modify affinity for the antigen (e.g., the targetable antigen or cell-surface protein) as necessary. For example, complementarity-determining regions (CDRs) can be derived from one source organism and combined with other components of another, such as human, to produce a chimeric molecule.

Production of antibodies or antibody-like molecules can be accomplished using any technique commonly known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. Bispecific antibodies can incorporate CDR regions of two different identified monoclonal antibodies by fusing encoding gene portions for the relevant binding domains followed by cloning into an expression vector that also comprises nucleic acids encoding the remaining structure(s) of the bispecific molecule.

Antibody fragments that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art.

The affinity reagent employed as the agent can also be an aptamer. As used herein, the term “aptamer” refers to oligonucleic or peptide molecules that can bind to specific antigens of interest. Nucleic acid aptamers usually are short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamers are thioaptamers, in which some or all of the non-bridging oxygen atoms of phosphodiester bonds have been replaced with sulfur atoms, which increases binding energies with proteins and slows degradation caused by nuclease enzymes. In some embodiments, nucleic acid aptamers contain modified bases that possess altered side-chains that can facilitate the aptamer-antigen binding.

Peptide aptamers are protein molecules that often contain a peptide loop attached at both ends to a protein scaffold. The loop typically is between 10 and 20 amino acids long, and the scaffold is typically any protein that is soluble and compact. One example of the protein scaffold is Thioredoxin-A, wherein the loop structure can be inserted within the reducing active site. Peptide aptamers can be generated/selected from various types of libraries, such as phage display, mRNA display, ribosome display, bacterial display and yeast display libraries.

The affinity reagents can be configured to carry a toxic payload that is detrimental to the cell with induced expression of the targetable antigen or cell surface protein. Alternatively, the affinity reagent can be configured to induce an immune response against the cell with induced expression of the targetable antigen or cell-surface protein.

In some embodiments, the second therapeutic composition comprises an antibody, or a fragment or derivative thereof. In other embodiments, the second therapeutic composition comprises an immune cell expressing an antibody, or fragment or derivative thereof, or an immune cell expressing a T cell receptor, or fragment or derivative thereof. The expressed antibody or T cell receptor, or fragment or derivative thereof, specifically binds the antigen. For example, in some embodiments, the immune cell expresses a chimeric antigen receptor with an antigen-binding domain and an intracellular domain that induces a response by the immune cell upon binding of the antigen-binding domain to the antigen or cell-surface receptor whose expression is selectively induced in the cancer cell.

In some embodiments, the functional therapeutic protein is a toxin. Any toxin that is locally detrimental or lethal to the expressing cell is encompassed by this disclosure. Some non-limiting examples include Caspase 9, TRAIL, Fas ligand, and the like, or functional fragments thereof.

In some embodiments, the functional therapeutic protein is a druggable enzyme. A druggable enzyme is an enzyme that is ideally not substantially prevalent in healthy cells, but when expressed presents a target for a known therapeutic, which can be additionally administered to the specific detriment of the cancer cell expressing the druggable enzyme target. Various druggable enzymes and their associated therapeutics are known and are encompassed by this disclosure. Non-limiting examples are provided below.

In one embodiment, the druggable enzyme is herpes simplex virus thymidine kinase and the method further comprises administering to the subject an effective amount of ganciclovir. For example, as described in the Examples below, a CDS for herpes simplex virus thymidine kinase (HSV-TK) was divided by the disclosed artificial introns in an expression cassette. When transcribed and properly spliced in leukemic or other cancer cells with a change of function mutation in the RNA splicing factor gene SRSF2, the exons are combined in the mRNA leading to proper expression of the HSV-TK protein in the cells. Upon treatment with ganciclovir, the cells are selectively killed compared to cells not properly expressing the HSV-TK (i.e., cell not receiving the expression cassette or cells with receiving the expression cassette but having wild-type SRSF2). Exemplary HSV-TK exon sequences that can be used in conjunction with the disclosed artificial introns to implement such cell-specific expression of functional HSV-TK protein are set forth as SEQ ID NOS: 19 and 20, and functional variants and derivatives thereof, are encompassed by the disclosure.

In one embodiment, the druggable enzyme is cytosine deaminase and the method further comprises administering to the subject an effective amount of 5-fluorocytosine. In one embodiment, the druggable enzyme is nitroreductase and the method further comprises administering to the subject an effective amount of CB1954 or analogs thereof. In one embodiment, the druggable enzyme is carboxypeptidase G2 and the method further comprises administering to the subject an effective amount of CMDA, ZD-2767P, and the like. In one embodiment, the druggable enzyme is purine nucleoside phosphorylase and the method further comprises administering to the subject an effective amount of 6-methylpurine deoxyriboside, and the like. In one embodiment, the druggable enzyme is cytochrome P450 and the method further comprises administering to the subject an effective amount of cyclophosphamide, ifosfamide, and the like. In one embodiment, the druggable enzyme is horseradish peroxidase and the method further comprises administering to the subject an effective amount of indole-3-acetic acid, and the like. In one embodiment, the druggable enzyme is carboxylesterase and the method further comprises administering to the subject an effective amount of irinotecan, and the like.

In some embodiments, the functional therapeutic protein is a detectable marker and can be useful in monitoring and/or guiding surgical procedures in the removal of the cancer cells. In some embodiments, the detectable marker provides a visual detectable signal (e.g., fluorescent signal) and the method further comprises surgically removing the cancer cells expressing the detectable marker. For example, as described in an Example below, a CDS for mEmerald was divided by the disclosed artificial introns in an expression cassette. When transcribed and properly spliced in target cancer cells with a change of function mutation in the RNA splicing factor gene SRSF2, the exons are combined in the mRNA leading to proper expression of the mEmerald protein in the cells. As a result, the cells are selectively fluorescent compared to cells not properly expressing the mEmerald protein (i.e., cell not receiving the expression cassette or cells with receiving the expression cassette but having wild-type SRSF2). Exemplary mEmerald exon sequences that can be used in conjunction with the disclosed artificial introns to implement such cell-specific expression of functional mEmerald protein are set forth below. Use of these disclosed exons in an expression cassette with the disclosed artificial intron constructs, and functional variants and derivatives thereof, are encompassed by the disclosure.

In yet other embodiments, multiple therapeutic proteins are simultaneously expressed. For example, as described in the Examples below, a CDS for herpes simplex virus thymidine kinase (HSV-TK) was divided by the disclosed artificial introns in an expression cassette. When transcribed and properly spliced in leukemic and other cancer cells with a change of function mutation in the RNA splicing factor gene SRSF2, the HSV-TK exons are combined in the mRNA leading to proper expression of the HSV-TK protein. Exemplary HSV-TK exon sequences that can be used in conjunction with the disclosed artificial introns to implement such cell-specific expression of functional HSV-TK protein are set forth below. Sequences for P2A CDS that can be used to implement such cell-specific expression are well known in the art and in addition to the sequence used here P2A CDSs (e.g., from foot-and-mouth disease virus, equine rhinitis A virus, Thosea asigna virus, and porcine teschovirus-1) are known and can be used. An exemplary IL-2 CDS is set forth in the Examples. Use of these disclosed sequences, individually or in combination, in an expression cassette with the disclosed artificial intron constructs, and functional variants and derivatives thereof, are encompassed by the disclosure.

The therapeutic compositions and/or additional therapeutic agents described herein can be formulated for any local or systemic mode of administration to facilitate efficient delivery and, with respect to the disclosed therapeutic composition with the artificial intron construct, expression in the target cells.

In some embodiments, the artificial nucleic acid intron construct, and expression cassette comprising a coding sequence (CDS) interrupted by at least one artificial nucleic acid intron, is comprised in a vector, e.g., viral expression vector, that facilitates expression of the heterologous nucleic acid in the nucleus of the target cell. In some embodiments, the vector promotes integration of the heterologous nucleic acid in the genome of the cell.

As indicated above, the construct may be present in a vector (e.g., a bacterial vector, a viral vector) or can be integrated into a genome. A “vector” is a nucleic acid molecule that is capable of transporting another nucleic acid molecule. Vectors can be, for example, plasmids, cosmids, viruses, an RNA vector or a linear or circular DNA or RNA molecule that can include chromosomal, non-chromosomal, semi-synthetic or synthetic nucleic acid molecules. Exemplary vectors are those capable of autonomous replication (episomal vector) or expression of nucleic acid molecules to which they are linked (expression vectors).

Viral vectors include retrovirus, adenovirus, parvovirus (e.g., adeno-associated viruses (AAV)), adenovirus, coronavirus, Newcastle disease virus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (see, e.g., Coffin, J. M., Retroviridae: The viruses and their replication, In Fundamental Virology, Third Edition, B. N. Fields et al., Eds., Lippincott-Raven Publishers, Philadelphia, 1996, incorporated herein by reference in its entirety).

As used herein, “expression vector” refers to a DNA construct containing a nucleic acid molecule that is operatively-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host cell, the vector may replicate and function independently of the host genome, or may, in some instances, integrate into the genome itself. In the present specification, “plasmid,” “expression plasmid,” “virus” and “vector” can be used interchangeably.

In some embodiments, the therapeutic composition further comprises a vehicle for intracellular delivery and a pharmaceutically acceptable carrier. The vehicle can be a liposome, nanocapsule, nanoparticle, exosome, microparticle, microsphere, lipid particle, vesicle, and the like, configured for the introduction of the expression cassette into cancer cells.

In some embodiments, the therapeutic composition further comprises a non-viral gene editing system and a pharmaceutically acceptable carrier. Chromosomal editing can be performed using, for example, endonucleases. As used herein, “endonuclease” refers to an enzyme capable of catalyzing cleavage of a phosphodiester bond within a polynucleotide chain. In certain embodiments, an endonuclease can be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule, such as the artificial synthetic introns herein, can be used for a donor gene “knock-in”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a FokI endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double-strand break (DSB) in the genome, and targeted integration of a transgene comprising flanking sequences homologous to the genome at the site of DSB is facilitated by homology-directed repair. Alternatively, a DSB generated by a ZFN can result in knock out of a target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation, or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histidine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entireties). TALENs can be used to direct site-specific double-strand breaks (DSBs) in the genomes of cells. Non-homologous end joining (NHEJ) ligates DNA from both sides of a double-strand break in which there is little, or no, sequence overlap for annealing, thereby introducing errors that knock out gene expression. Alternatively, homology-directed repair can introduce a transgene at the site of DSB, providing homologous flanking sequences are present in the transgene. In certain embodiments, a gene knockout comprises an insertion, a deletion, a mutation, or a combination thereof, made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. The type II system, the most studied, comprises at least three components: an RNA-guided Cas9 nuclease, a crRNA, and a trans-acting crRNA (tracrRNA). The tracrRNA comprises a duplex-forming region. A crRNA and a tracrRNA form a duplex that is capable of interacting with a Cas9 nuclease and guiding the Cas9/crRNA: tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology-directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programmed to target a desired sequence (Xie et al., PLOS One 9: e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference in its entirety). In certain embodiments, a gene knockout comprises an insertion, a deletion, a mutation, or a combination thereof, made using a CRISPR/Cas nuclease system.

As used herein, a “meganuclease,” also referred to as a “homing endonuclease,” refers to an endodeoxyribonuclease characterized by a large recognition site (double-stranded DNA sequences of about 12 to about 40 base pairs). Meganucleases can be divided into five families based on sequence and structure motifs: LAGLIDADG, GIY-YIG, HNH, His-Cys box and PD-(D/E)XK. Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nuc'l. Acids Res. 25:3379-3388, 1997; Dujon et al., Gene 82:115-118, 1989; Perler et al., Nuc'l. Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998, each of which is incorporated herein by reference in its entirety).

As indicated above, the CDS generated by splicing the artificial intron can be a protein that provides a detectable signal. The selective expression of such a reporter protein in a cancer cell can be leveraged to guide more specific and targeted surgical techniques. Accordingly, in another aspect, the disclosure provides a method of enhancing surgical resection of a tumor from a subject. In this aspect, the tumor is characterized by a change-of-function or loss-of-function mutation in a recurrently mutated RNA splicing factor gene. The method comprises administering to the subject an effective amount of a therapeutic composition comprising an expression cassette comprising a coding sequence (CDS) encoding a detectable marker, wherein the CDS is interrupted by at least one artificial nucleic acid intron as described above, and wherein the expression cassette further comprises a promoter operatively linked to the CDS.

In one embodiment, the RNA splicing factor gene is SRSF2. Cancer types associated with recurrent change-of-function mutations in RNA splicing factor genes, such as SRSF2, are known and are encompassed by this aspect of the disclosure. Exemplary cancer types include uveal melanoma, mucosal melanoma, skin melanoma, breast cancer, pancreatic cancer, endometrial cancer, liver cancer, lung cancer, mesothelioma, or other solid tumor or neoplasm with recurrent SRSF2 mutations.

In some embodiments, the detectable marker is a fluorescent or luminescent protein. For example, any fluorescent protein at any detectable spectrum (blue/uv, cyan, green, yellow, orange, red, far-red, and the like) can be used. See, e.g., Snapp E. Design and use of fluorescent fusion proteins in cell biology. Curr Protoc Cell Biol. 2005; Chapter 21:21.4.1-21.4.13. doi:10.1002/0471143030.cb2104s27, incorporated herein by reference in its entirety. Non-limiting examples of fluorescent and luminescent proteins include TagBFP2, BFP, mTurquoise2, TagGFP2, GFP, eGFP, Superfolder GFP, TurboGFP, mEmerald, Azamin Green, mTFP1 (Teal), EYFP, Topaz, T-Sapphire, mWasabi, m Venus, mKO, EBFP, ABFP2, Azurite, mTagBFP, ECFP, Cerulean, mTurquoise, CyPet, AmCyan1, Midori-Ishi Cyan, TagCFP, mCitrine, YPet, TagYFP, PhiYFP, ZsYellow1, mBanana, mOrange, dTomato, TagRFP, DsRed/2, mTangerine, mRuby, mStrawberry, Jred, mRaspberry, mPlum, mApple, mCherry, mKate2, Katushka, mCardinal, firefly luciferase, renilla luciferase, and the like.

The method can further comprise the step of detecting fluorescent or luminescent tumor cells and surgically resecting the fluorescent or luminescent tumor cells.

The expression cassette can be disposed in a vector, e.g., a viral vector, or otherwise formulated with a vehicle (e.g., nanoparticle, liposome, and the like) for intracellular delivery, as described above in more detail.

In another aspect, the disclosure provides an in vitro method of screening candidate compositions for activity in a cell. The cell has a genetic background comprising a change-of-function or loss-of-function mutation in a recurrently mutated RNA splicing factor gene. The cells can be established transformed cell lines with known genetic backgrounds or can be cells derived from a subject with a suspected genetic background that comprises a change-of-function or loss-of-function mutation in a recurrently mutated RNA splicing factor gene. For example, in some embodiments, the RNA splicing factor gene is SRSF2.

The method comprises contacting the cell with an expression cassette comprising a coding sequence (CDS) interrupted by at least one artificial nucleic acid intron, as disclosed herein. The cell is contacted with a candidate composition and transcription from the expression cassette, with any transcriptional processing (i.e., RNA splicing), is permitted. The cells are monitored for modulation of the expression of a functional reporter protein, which indicates whether the candidate composition modulates the activity of the recurrently mutated RNA splicing factor. In some embodiments, the modulation is the presence or increase of functional reporter protein when a mutated RNA splicing factor is present and functionally active. In alternative embodiments, the modulation is the decrease or absence of functional reporter protein in when a mutated RNA splicing factor is present and functionally active.

The expression cassette can comprise a promoter and/or appropriate enhancers operatively linked to the CDS. Upon processing of the transcript encoded, and potential splicing of the artificial nucleic acid intron, the CDS encodes or does not encode a functional detectable reporter protein. Splicing depends upon mutant splicing factor activity in the cell and, therefore, differs between cells with a genetic background comprising a change-of-function or loss-of-function mutation in a recurrently mutated RNA splicing factor gene and cells lacking such a mutation.

For example, as described in the Examples below, a CDS for mEmerald was divided by the disclosed artificial intron in an expression cassette. When transcribed and properly spliced in breast epithelial and melanoma cells, which possess a change of function mutation in the RNA splicing factor gene SRSF2, the exons were combined in the mRNA leading to expression of intact mEmerald protein by the cells. In contrast, cells lacking such a mutation in SRSF2 did not express mEmerald, which replicated the effect of a compound that successfully modulates (i.e., inhibits) the activity of the recurrently mutated RNA splicing factor. This difference between cells with or without aberrant SRSF2 splicing activity is readily detectable as a difference in the relative fluorescence signal.

In some embodiments, detection of a functional reporter protein or a relative increase of functional reporter protein in the cell indicates the candidate composition does not suppress activity of the mutated RNA splicing factor in the cell. In contrast, detection of an absence or relative reduction in functional reporter protein in the cell indicates the candidate composition does suppress activity of the mutated RNA splicing factor in the cell.

The screen can be scaled up to assess the impact of a library of candidate compounds on aberrant RNA splicing due to change-of-function or loss-of-function mutation(s) in a recurrently mutated RNA splicing factor gene. The screen can be characterized as a positive screen, i.e., assessing for a positive effect in inhibiting aberrant RNA splicing. In some embodiments, indicated above, the cells are derived from a subject, e.g., from a biopsy. The screen can be implemented to assess how the suspected cancer in the subject might respond to a variety of candidate therapeutics. For example, the cells can be expanded and arranged in an array plate and individual cells, or groups of cells, are transformed with the expression cassette comprising the artificial intron and contacted with different potential therapeutics. In some embodiments, the detection of reporter protein is indicative of the aberrant splicing activity and, thus, is inversely proportional to the efficacy of the therapeutic contacted to the cells.

In other embodiments, the screen can be characterized as a negative screen. The expression cassette comprising the synthetic intron can be configured, as described above, to preferentially result in expression of a functional reporter protein in the absence of a mutated RNA splicing factor or in the presence of an inhibited mutated RNA splicing factor. Accordingly, detection of a functional reporter protein in the cell indicates the candidate composition suppresses activity of the mutated RNA splicing factor in the cell. In contrast, an absence or relative reduction in detected functional reporter protein in the cell indicates the candidate composition does not suppress activity of the mutated RNA splicing factor in the cell.

The step of detecting the presence of a functional reporter protein can comprise quantifying the amount of reporter protein. This can be performed according to standard techniques in the art and depends on the nature of the reporter protein incorporated into the method.

Reporter proteins, and their sequences, appropriate for these methods are well-known in the art and are encompassed by the present disclosure. A nonlimiting list of exemplary reporter proteins are described above. In some embodiments, the reporter protein is a fluorescent protein or a luminescent protein. Other reporter proteins can be enzymatic proteins, such as β-galactosidase, that catalyze reactions that can be readily assayed.

In some embodiments, the method further comprises contacting a control cell without a change-of-function or loss-of-function mutation in a recurrently mutated RNA splicing factor gene with the expression cassette and further contacting the control cell with the candidate composition. This can provide a reference or standard reporter protein level to which the experimental screen results can be compared.

The candidate composition can be any composition suspected of having a potential direct or indirect effect on the transcription or splicing functionality in a cell. For example, the candidate composition can be selected from a small molecule, protein (e.g., antibody, or fragment or derivative thereof, enzyme, and the like), and nucleic acid construct to alter the genome or transcriptome of the cell, or a complex of a nucleic acid and protein.

In some embodiments, the nucleic acid construct is an interfering RNA construct. In other embodiments, the candidate composition comprises a Transcription Activator-Like Effector Nuclease (TALEN), Zinc Finger Nuclease (ZFN), or recombinant fusion protein. In some embodiments, the candidate composition comprises a guide nucleic acid specific for a target sequence and an associated nuclease that modifies and/or cleaves a nucleic acid molecule upon binding of the guide nucleic acid to its target sequence. Alternatively, the candidate composition comprises a guide nucleic acid specific for a target sequence and an associated catalytically inactive nuclease, wherein binding of the guide nucleic acid to the target sequence results in modification of transcription, splicing, or translation of the target sequence. In further embodiments, the associated nuclease is Cas9, Cas12, Cas13, Cas14, variants thereof, and the like.

In a similar aspect, the disclosure provides a method of screening a cell with suspected genetic background comprising a change-of-function or loss-of-function mutation in a recurrently mutated RNA splicing factor gene. The cell can be derived from a subject, e.g., a suspected cancer cell obtained from the subject. As above, the cell is contacted with an expression cassette comprising a coding sequence (CDS) interrupted by at least one artificial nucleic acid intron, as disclosed herein. The cell is monitored for expression of an intact protein resulting from a complete CDS, e.g., an intact reporter protein, which indicates aberrant activity of an RNA splicing factor and, thus, indicates the presence of a change-of-function or loss-of-function mutation in a recurrently mutated RNA splicing factor gene, as described above. Cells that exhibit aberrant RNA splicing, as indicated by presence of a protein encoded by the CDS, can be further subjected to a screen of candidate compounds that may inhibit aberrant RNA splicing to determine the appropriateness of the candidate compounds as a therapeutic.

### Additional Definitions

Unless specifically defined herein, all terms used herein have the same meaning as they would to one skilled in the art of the present invention. Practitioners are particularly directed to Sambrook J., et al., (eds.), Molecular Cloning: A Laboratory Manual, 3rd ed., Cold Spring Harbor Press, Plainsview, New York (2001); Ausubel, F. M., et al., (eds.), Current Protocols in Molecular Biology, John Wiley & Sons, New York (2010); and Coligan, J. E., et al. (eds.), Current Protocols in Immunology, John Wiley & Sons, New York (2010) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having cancer. While subjects may be human, the term also encompasses other mammals, particularly those mammals useful as laboratory models for human disease, e.g., mouse, rat, dog, non-human primate, and the like.

The term “treating,” and grammatical variants thereof can refer to any indicia of success in the treatment or amelioration or prevention of a disease or condition (e.g., a cancer, infectious disease, or autoimmune disease), including any objective or subjective parameter such as abatement; remission; diminishing of symptoms or making the disease condition more tolerable to the patient; slowing in the rate of degeneration or decline; or making the final point of degeneration less debilitating.

The treatment or amelioration of symptoms can be based on objective or subjective parameters, including the results of an examination by a physician. Accordingly, the term “treating” includes the administration of the compounds or agents of the present disclosure to prevent or delay, to alleviate, to improve clinical outcomes, to decrease occurrence of symptoms, to improve quality of life, to lengthen disease-free status, to stabilize, to prolong survival, to arrest or inhibit development of the symptoms or conditions associated with a disease or condition (e.g., a cancer), or any combination thereof. The term “therapeutic effect” refers to the reduction, elimination, or prevention of the disease or condition, symptoms of the disease or condition, or side effects of the disease or condition in the subject.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds, or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:
(1) Alanine (A), Serine(S), Threonine (T),
(2) Aspartic acid (D), Glutamic acid (E),
(3) Asparagine (N), Glutamine (Q),
(4) Arginine (R), Lysine (K),
(5) Isoleucine (I), Leucine (L), Methionine (M), Valine (V), and
(6) Phenylalanine (F), Tyrosine (Y), Tryptophan (W).

As used herein, the term “nucleic acid” refers to a polymer of nucleotide monomer units or “residues”. The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

Reference to sequence identity addresses the degree of similarity of two polymeric sequences, such as nucleic acid or protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention and are not intended to limit the scope of what the inventors regard as their invention nor are they intended to represent that the experiments below are all or the only experiments performed.

### Example 1

### Experimental Strategy

Endogenous introns in the human genome that were spliced differently in primary samples from patients with myeloid malignancies with or without SRSF2 mutations were identified. Shorter synthetic versions of these introns were created by removing sequences that were not essential for responding to SRSF2 mutations, inserting the synthetic intron into open reading frames encoding proteins of interest (e.g., fluorescent proteins or cytokines), and identifying synthetic introns that yielded SRSF2 mutation-dependent protein production.

### Results

An exemplar synthetic intron was created that responded specifically to SRSF2 mutations. This synthetic intron was derived from an endogenous region of the human MELK gene. This synthetic intron consists of three contiguous components (Table 1): an approximately 250 nt intron that is derived from intron 12 of the endogenous MELK gene (See, for example, SEQ ID NO:1), an approximately 123 nt alternatively spliced (“cassette”) exon that is derived from exon 12 of the endogenous MELK gene (See, for example, SEQ ID NO:2), and an approximately 250 nt intron that is derived from intron 13 of the endogenous MELK gene (See for example, SEQ ID NO:3). It should be noted that the term “synthetic intron” refers to all three components. Additional key features of the cassette exons were identified that were important for the response to SRSF2 mutations, particularly, the presence of GGNG (N=any nucleotide) motifs that were previously shown to be associated with reduced exon recognition in cells with an SRSF2 mutation relative to wildtype (WT) cells due to reduced binding of GGNG motifs by mutant SRSF2 relative to WT SRSF2. When inserted into an open reading frame encoding a protein of interest, this synthetic intron has the property of yielding more protein in SRSF2-mutant cells than in WT cells. This difference in protein production arises because SRSF2-mutant cells preferentially skip the cassette exon within the synthetic intron construct, whereas WT cells preferentially include the cassette exon. Inclusion of the cassette exon disrupts the open reading frame. Therefore, SRSF2-mutant cells preferentially produce mature mRNA with a disrupted open reading frame that does not encode the protein of interest.

This synthetic intron was inserted into open reading frames encoding various proteins of interest.

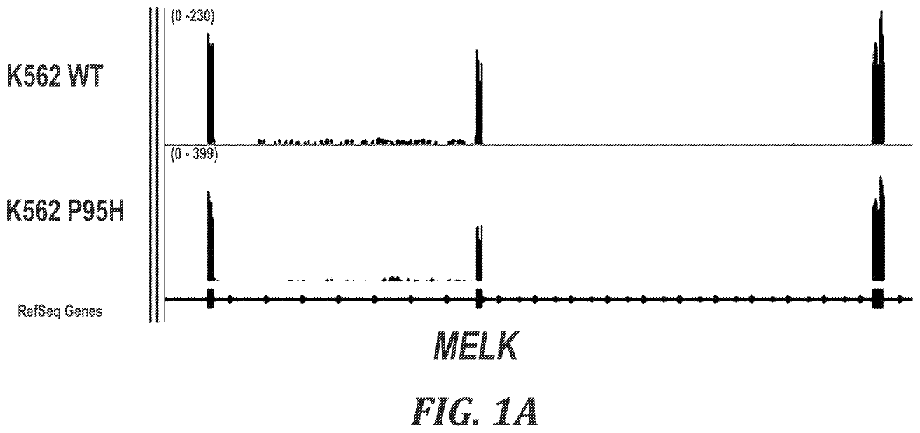

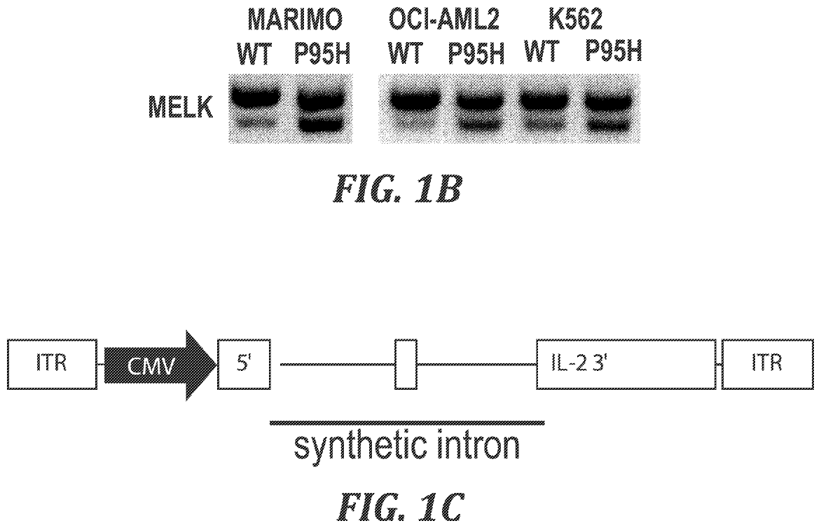

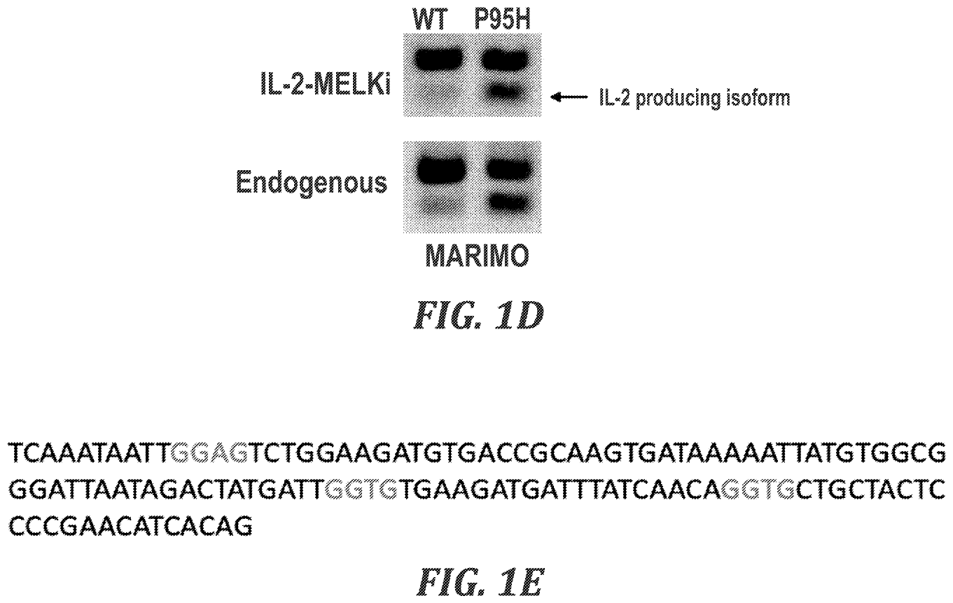

The open reading frames included, IL-2 (See Table 1 for the flanking exon sequences), mEmerald (See Table 1 for the flanking exon sequences), and HSV-TK (See Table 1 for the flanking exon sequences). The resulting constructs can be used to specifically produce a cytokine, fluorescent protein, or druggable enzyme in SRSF2-mutant cells, while leaving WT cells unaffected. See FIGS. 1A-1E.

TABLE 1
Nucleotide Sequences for Components of Exemplar Synthetic Intron that
Responded Specifically to SRSF2 Mutations

| | | |
|---|---|---|
| synMELKi12-250 | gtaagtgttactgcctgttgtgtcttgccacgtgccctccctgttc | SEQ ID NO: 1 |
| | ctgacagcctaggtgtggcctaggtgtgtagtaggctctgacgt | |
| | ctcagtttgtgtaagtacattttatgatgttcacacagtgataaa | |
| | attgcctaatacatttctcagaatgtgtctctgtcgttaagtgac | |
| | gcgtgactgtattttaaatattgaatcatagtactgtcatctttaa | |
| | gttcttctgtctttcattgagtag | |
| synMELKe12 | tcaaataattggagtctggaagatgtgaccgcaagtgataa | SEQ ID NO: 2 |
| | aaattatgtggcgggattaatagactatgattggtgtgaagatg | |
| | atttatcaacaggtgctgctactccccgaacatcacag | |
| synMELKi130250 | gttcgtcattcttattactatttaaggcagaatctattgtttcaatt | SEQ ID NO: 3 |
| | atatgtcaatagaacagactaaagtgatctacatgtattaaag | |
| | aaaactatatattttcaaatttcctaatgtcaataatatccttttga | |
| | taattcattttaataaaaatagtacaaggtagtttgcaaatgatc | |
| | aaggaatgttgtaaagaaattgacttttcttcttcagtgtgcttta | |
| | tctagtaactttttttttccag | |
| IL2e1-synMELK | atgtacag | |
| IL2e2-synMELK | gatgcaactcctgtcttgcattgcactaagtcttgcacttgtcac | SEQ ID NO:4 |
| | aaacagtgcacctacttcaagttctacaaagaaaacacagc | |
| | tacaactggagcatttactgctggatttacagatgattttgaat | |
| | ggaattaataattacaagaatcccaaactcaccaggatgctc | |
| | acagccaagttttacatgcccaagaaggccacagaactga | |
| | aacatcttcagtgtctagaagaagaactcaaacctctggagg | |
| | aagtgctaaatttagctcaaagcaaaaactttcacttcgaccc | |
| | cagggacgtggtgagcaatatcaacgtattcgttctggaact | |
| | aaagggatctgaaacaacattcatgtgtgaatatgctgatga | |
| | gacagcaaccattgtagaatttctgaacagatggattaccttt | |
| | gtctcaaagcatcatctcaacactgacttga | |
| HSV-TKe1- | atggcttcgtacccctgccatcaacacgcgtctgcgttc | SEQ ID NO: 5 |
| synMELK | gaccaggctgcgcgttctcgcggccatagcaaccgac | |
| | gtacggcgttgcgccctcgccggcagcaaggaagcca | |
| | cggaagtccgcctggagcagaaaatgcccacgctactg | |
| | cgggtttatatagacggtcctcacgggatggggaaaac | |
| | caccaccacgcaactgctggtggccctgggttcgcgc | |
| | gacgatatcgtctacgtacccgagccgatgacttactgg | |
| | caggtgctgggggcttccgagacaatcgcgaacatcta | |
| | caccacacaacaccgcctcgaccagggtgagatatc | |
| | ggccgg | |
| HSV-TKe2- | ggacgcggcggtggtaatgacaagcgcccagataac | SEQ ID NO: 6 |
| synMELK | aatgggcatgccttatgccgtgaccgacgccgttctgg | |
| | ctcctcatatcgggggggaggctgggagctcacatgc | |
| | cccgcccccggccctcaccctcatcttcgacctccatcc | |
| | catcgccgccctcctgtgctacccggccgcgcgatacc | |
| | ttatgggcagcatgaccccccaggccgtgctggcgttcg | |
| | tggccctcatcccgccgaccttgcccggcacaaacatc | |
| | gtgttgggggcccttccggaggacagacacatcgacc | |
| | gcctggccaaacgccagcgccccggcgagcggcttg | |
| | acctggctatgctggccgcgattcgccgcgtttacggg | |
| | ctgcttgccaatacggtgcggtatctgcagggcggc | |
| | gggtcgtggcgggaggattggggacagctttcggg | |
| | gacggccgtgccgccccagggtgccgagcccca | |
| | gagcaacgcgggcccacgaccccatatcggggaca | |
| | cgttatttaccctgtttcgggcccccgagttgctggcc | |
| | cccaacggcgacctgtacaacgtgtttgcctgggcct | |
| | tggacgtcttggccaaacgcctccgtcccatgcacgt | |
| | ctttatcctggattacgaccaatcgcccgccggctgc | |
| | cgggacgccctgctgcaacttacctccgggatggtc | |
| | cagacccacgtcaccacccccggctccataccgacgat | |
| | ctgcgacctggcgcgcacgtttgcccgggagatgg | |
| | gggaggctaactga | |
| mEmeralde1- | atggtgagcaagggcgaggagctgttcaccggggt | SEQ ID NO: 7 |
| synMELK | ggtgcccatcctggtcgagctggacggcgacgtaa | |
| | acggccacaagttcagcgtgtccggcgagggcgag | |
| | ggcgatgccacctacggcaagctgaccctgaagttca | |
| | tctgcaccaccggcaagctgcccgtgccctggcccacc | |
| | ctcgtgaccaccttgacctacggcgtgcagtgcttcgccc | |
| | gctaccccgaccacatgaagcagcacgacttcttcaagt | |
| | ccgccatgcccgaaggctacgtccaggagcgcaccatc | |
| | ttcttcaaggacgacggcaactacaagacccgcgccgag | |
| | gtgaagttcgag | |
| mEmeralde2- | ggcgacaccctggtgaaccgcatcgagctgaagggcat | SEQ ID NO: 8 |
| synMELK | cgacttcaaggaggacggcaacatcctggggcacaagc | |
| | tggagtacaactacaacagccacaaggtctatatcaccgc | |
| | cgacaagcagaagaacggcatcaaggtgaacttcaaga | |
| | cccgccacaacatcgaggacggcagcgtgcagctcgcc | |
| | gaccactaccagcagaacacccccatcggcgacggccc | |
| | cgtgctgctgcccgacaaccactacctgagcacccagtcc | |
| | aagctgagcaaagaccccaacgagaagcgcgatcacatg | |
| | gtcctgctggagttcgtgaccgccgccgggatcactctcgg | |
| | catggacgagctgtacaagtaa | |

### Example 2

This Example describes a method to harness this abnormal splicing activity to drive splicing factor mutation-dependent gene expression in cancers and selectively eliminate these tumors. Synthetic introns were engineered that were efficiently spliced in cancer cells bearing SRFS2 mutations, but unspliced in otherwise isogenic wild-type cells, to yield mutation-dependent protein production. A massively parallel screen of introns delineated ideal intronic size and mapped essential sequence elements underlying mutation-dependent splicing. Synthetic introns enabled mutation-dependent expression of herpes simplex virus thymidine kinase and subsequent ganciclovir-mediated elimination of SRSF2-mutant cancer cells, while leaving wild-type cells unaffected. The modular, compact, and specific nature of synthetic introns provides a power platform for inducing gene expression in selectively in specific cancer cells. This can be leveraged, for example, as a means to exploit cancer-specific changes in RNA splicing for gene therapy, among other applications.

### Results and Discussion

Recurrent mutations affecting an RNA splicing factor occur in many cancer types, with frequencies ranging from about 40% in CMML and about 10% in non-RS MDS and 25% in acute myeloid leukemia (AML) in patient over 60 years of age and about 5% in AML is patients under 60 years of age. These lesions are attractive targets for therapeutic development thanks to their pan-cancer nature, frequent occurrence as initiating or early events, presence in the dominant clone, and particular enrichment in diseases with few effective therapies. Accordingly, several studies have demonstrated that cancer cells bearing spliceosomal mutations are preferentially sensitive to further splicing perturbation, including treatment with compounds that inhibit normal spliceosome assembly or function. However, the therapeutic index of drugs that inhibit global splicing activity is not yet clear. Moreover, therapeutic approaches that target the function of the mutant splicing machinery itself have not yet been identified.

Spliceosomal mutations alter splice site and exon recognition to cause dramatic mis-splicing of a restricted set of genes, while leaving most genes unaffected. Although these splicing changes promote aberrant self-renewal, transformation, and other pro-tumorigenic phenotypes, the inventors hypothesized that this splicing dysregulation could be exploited for therapeutic development. Accordingly, synthetic constructs were designed, developed, and tested for differential splicing in cells with or without recurrent mutations in SRSF2, (one of the most commonly mutated spliceosomal gene in cancer), to allow for cancer cell-specific protein production.

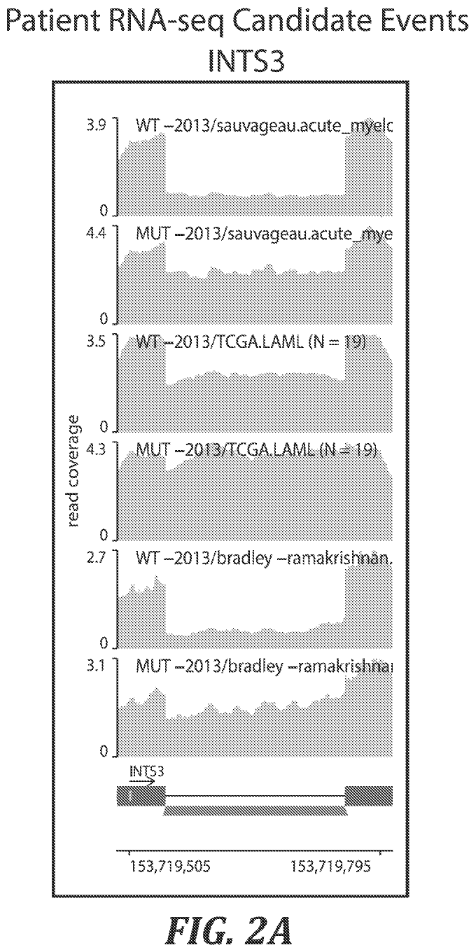

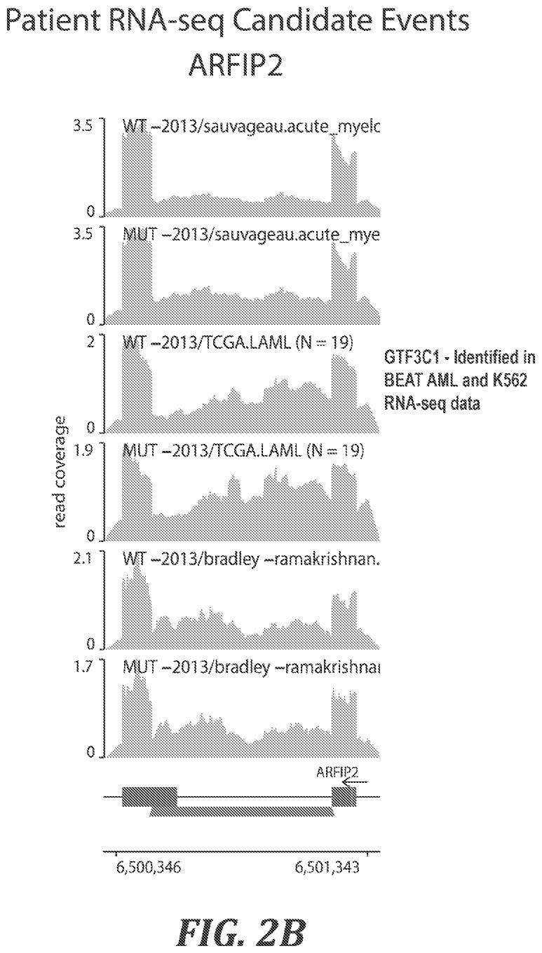

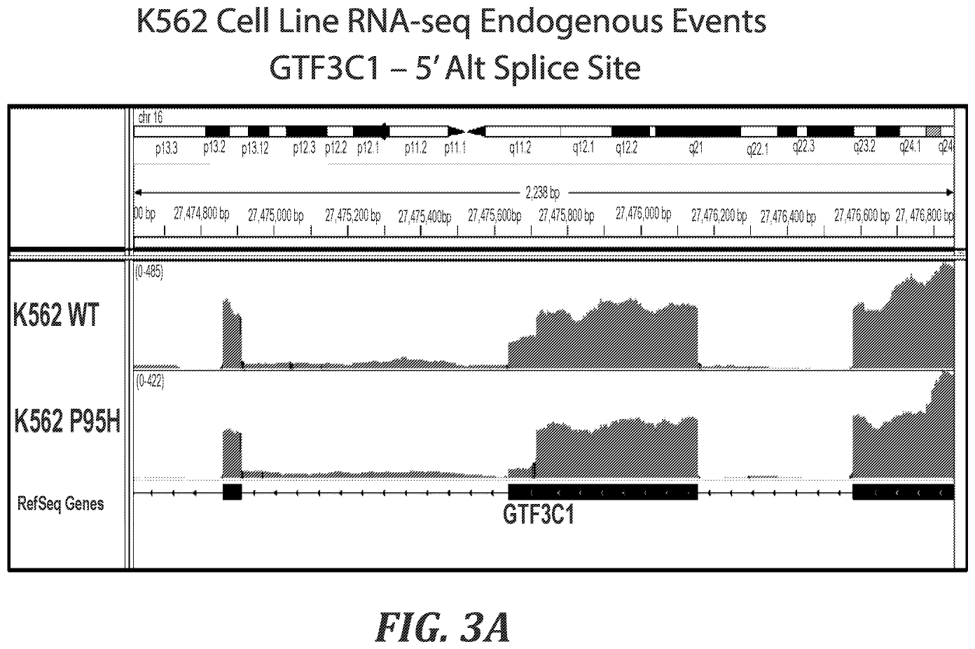

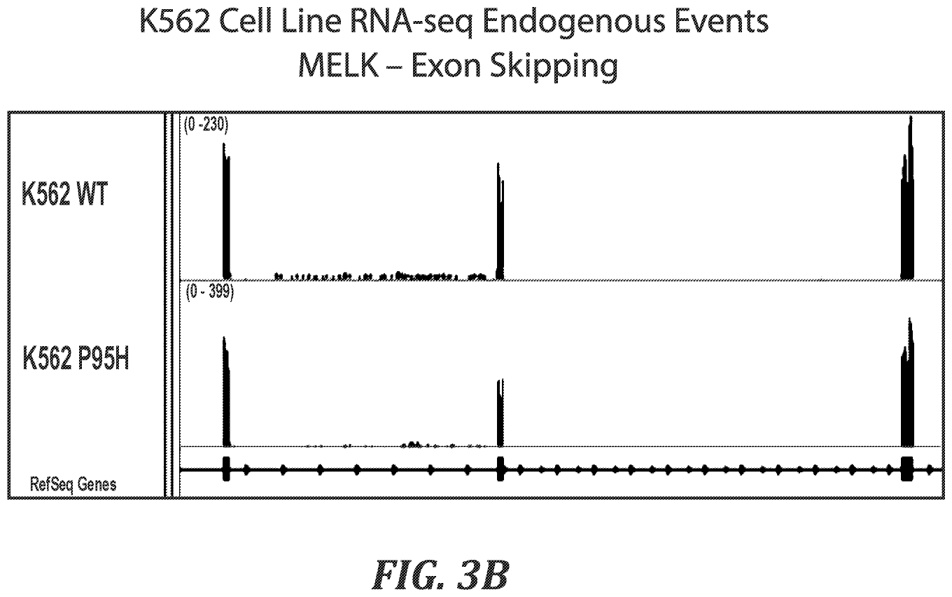

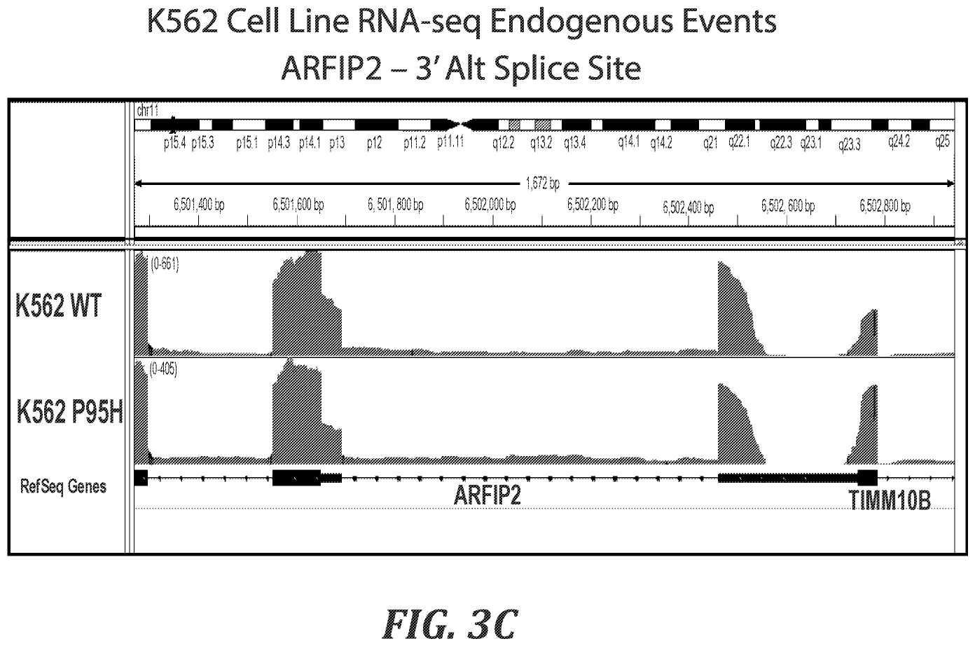

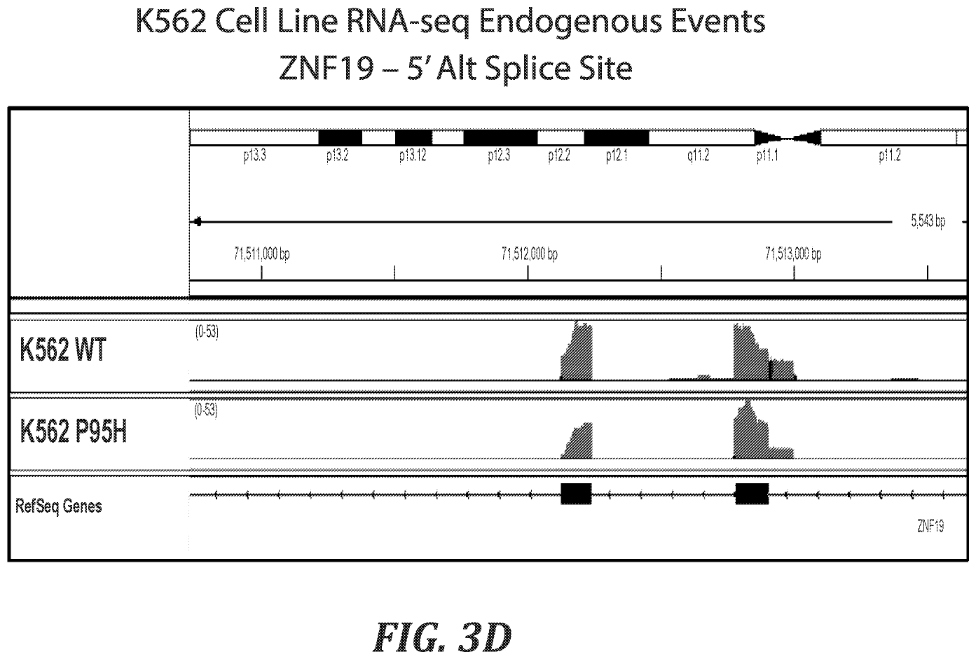

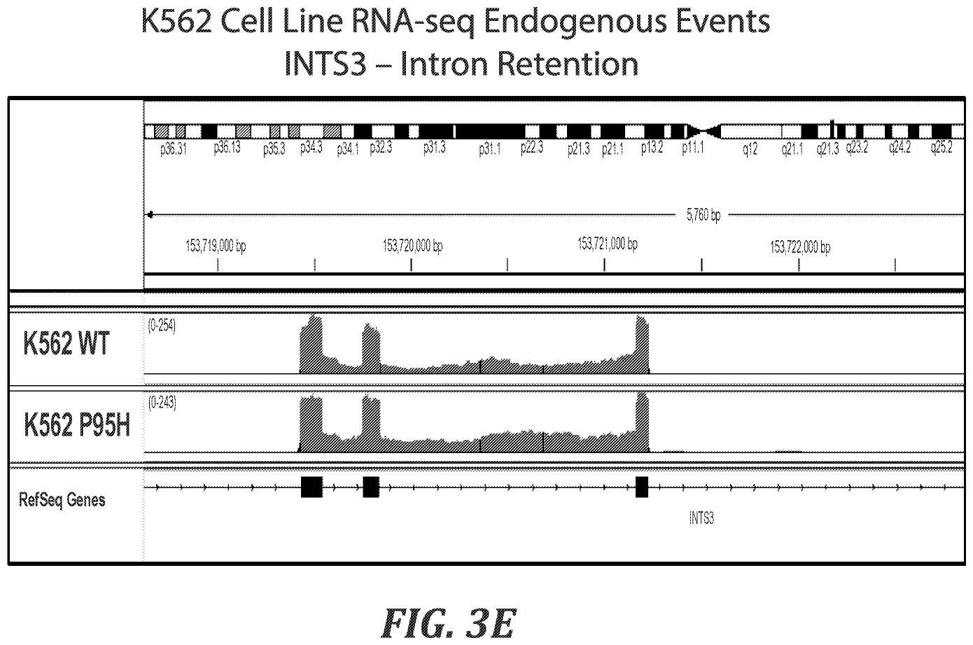

Endogenous genes were first identified that responded most strongly and consistently to SRSF2 mutations, which are near-universally present as heterozygous, missense changes affecting a few residues. SRSF2 wildtype and mutant patient RNA-sequencing data from 3 independent studies (Sauvageau AML data set (Lavallee et al., The transcriptomic landscape and directed chemical interrogation of MLL-rearranged acute myeloid leukemias, Nat. Genet. 47:1032-1037 (2015)); and the Bradley/Ramakrishnan data set (Kim et al., SRSF2 mutations contribute to myelodysplasia by mutant-specific effects on exon recognition, Cancer Cell 27 (5): 617-630 (2015)) were queried to identify intron retention events in the genes INTS3 and ARFIP2. FIG. 2A-2B. In addition, RNA-sequencing data from the isogenic cell line K562 SRSF2^{+/+} and SRSF2^{+/P95H} demonstrated alternative splicing events in the genes GTF3C1 (FIG. 3A), MELK (FIG. 3B), ARFIP2 (FIG. 3C), ZNF19 (FIG. 3D), and INTS3 (FIG. 3E). SRSF2 mutations were associated with diverse splicing changes, including altered 3′ splice site (3′ss) selection, exon recognition, and intron retention.

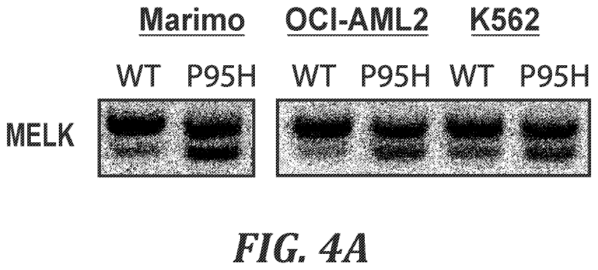

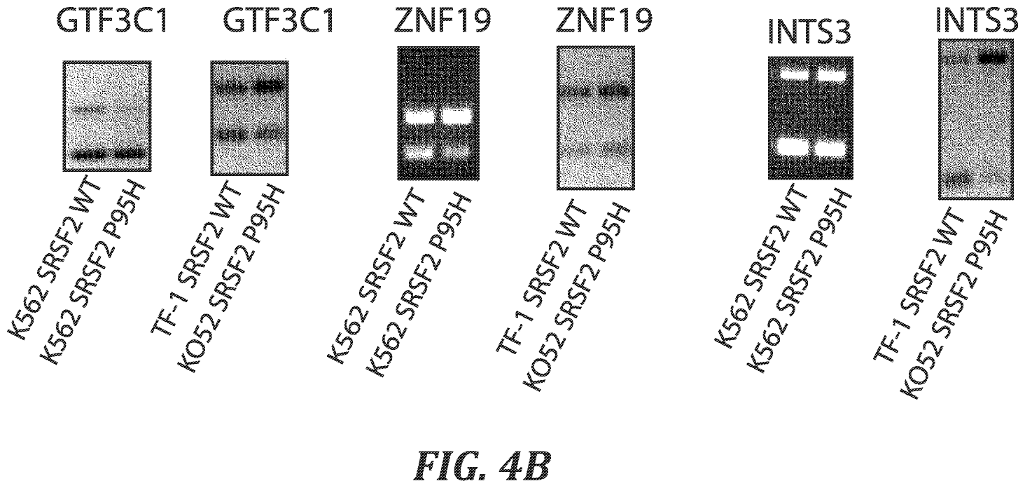

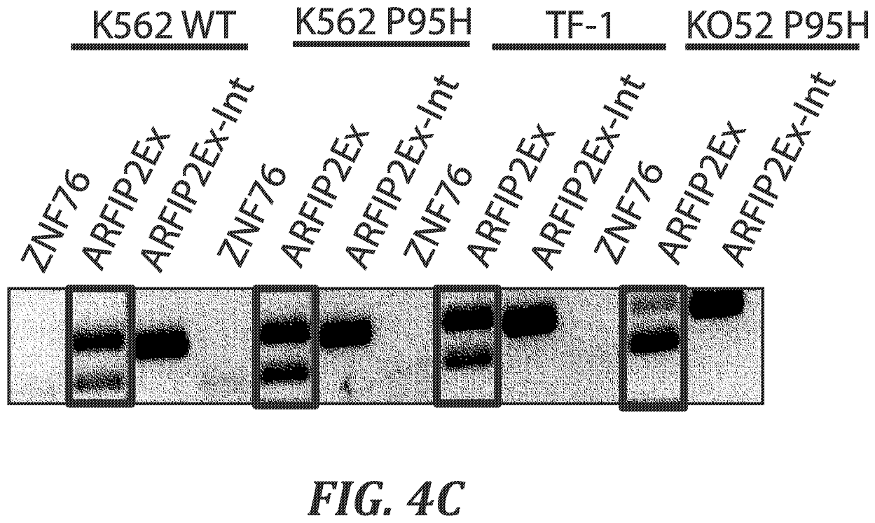

The identified endogenous alternative splicing events were confirmed via reverse transcriptase polymerase chain reaction (RT-PCR) products from various cells lines including WT SRSF2^{+/+}) and SRSF2^{+/P95H} isogenic cell lines MARIMO, OCI-AML2 and K562 (FIG. 4A), SRSF2^{+/+} or SRSF2^{+/P95H} isogenic cell line K562 or TF-1 (SRSF2^{+/+}) and KO52 (SRSF2^{+/P95H}) amplified using primers for endogenous alternative splicing events in the genes GTF3C1 (5′ alternative splice site), ZNF19 (3′ alternative splice site) and INTS3 (intron retention) (FIG. 4B); and SRSF2^{+/+} or SRSF2^{+/P95H} isogenic cell line K562 or TF-1 (SRSF2^{+/+}) and KO52 (SRSF2^{+/P95H}) amplified using primers for an endogenous alternative splicing event in the gene ARFIP2 (3′ alternative splice site) (FIG. 4C). These proof-of-principle studies confirmed the feasibility of using synthetic introns for mutation-dependent gene expression.

Because these endogenous introns were too long to be useful in gene therapy, shorter, synthetic versions of these endogenous introns were created by removing all sequence that were believed nonessential for SRSF2 mutation-dependent splicing. Additionally, a completely novel synthetic intron was designed and created termed MELK/GTF3C1_356 nt, which incorporates a combination of splicing elements utilized in the MELK and GTF3D1 synthetic introns.

Synthetic introns falling into the following 5 families were created:
Family 1: synMELK derived from intron 10, exon 10, and intron 11 of the human MELK gene;
Family 2: synGTF3C1 derived from intron 34 of the human GTF3C1 gene;
Family 3: synARFIP2 derived from intron 1 of the human ARFIP2 gene;
Family 4: synINTS3 derived from intron 4 and exon 5 of the human INTS3 gene; and
Family 5: synZNF19 derived from exon 3 and intron 3 of the human ZNF19 gene. Table 1. Nucleotide Sequence of the Endogenous Introns and Exons for the MELK, gtf3

Each of the mutation specific synthetic introns within each of the above families were created as described herein:

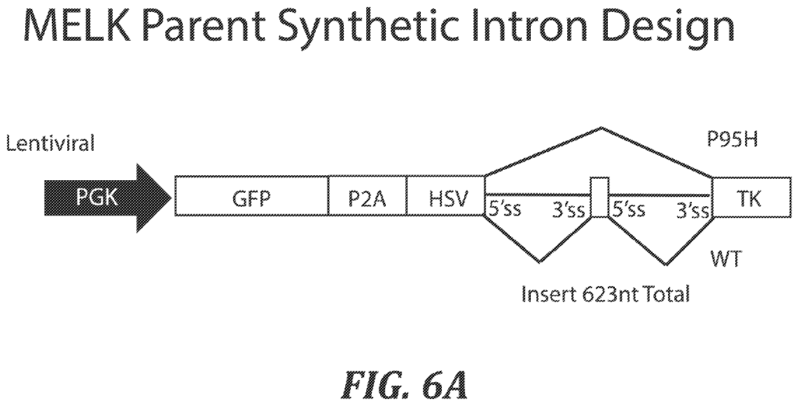

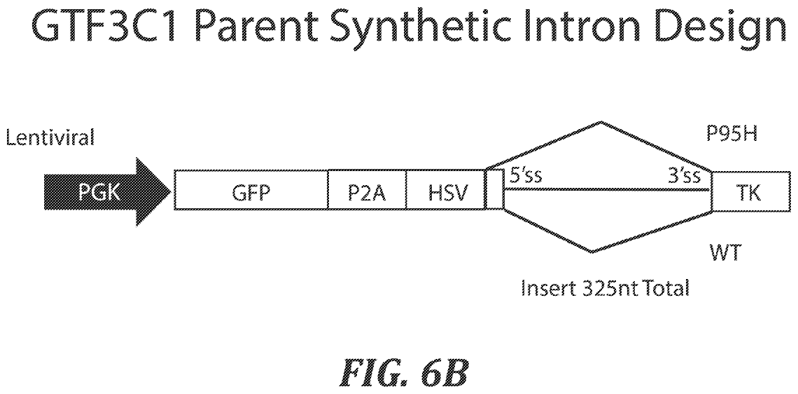

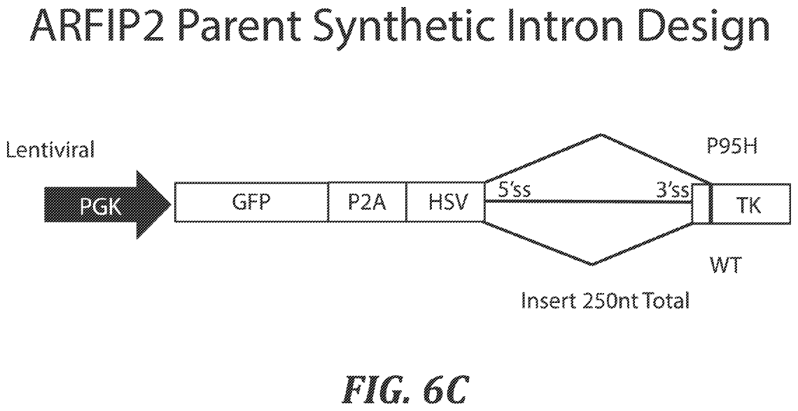

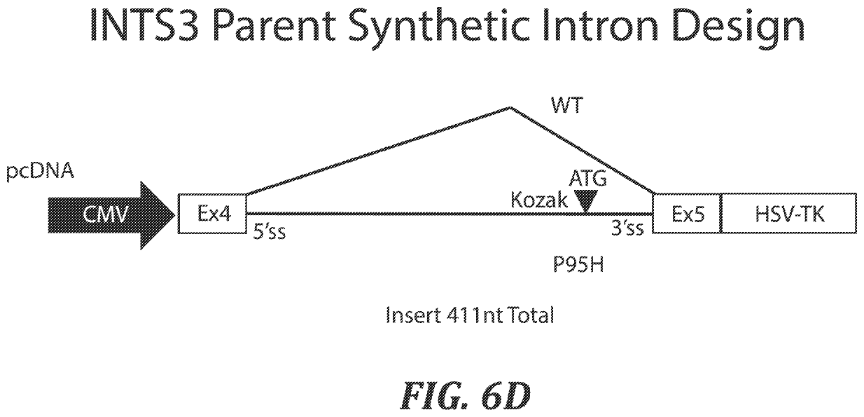

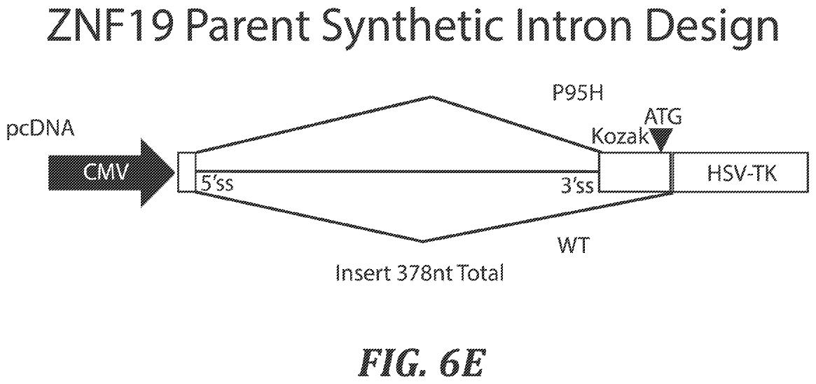

ParentSynMELK comprises a total of 623 nucleotides (nt). (FIG. 6A) The first 50 nt of MELK intron 10 which encompasses the endogenous 5′ splice site of intron 10 was ligated to the last 200 nt of MELK intron 10 which encompasses the endogenous 3′ splice site of intron 10. This sequence was ligated to endogenous MELK exon 10 (123 nt), This segment was ligated to the first 50 nt of MELK intron 11 which encompasses the endogenous 5′ splice site of intron 11 and was ligated to the last 200 nt of MELK intron 11 which encompasses the endogenous 3′ splice site of intron 11. Additional shortened variations of this parent synthetic intro were created a per the Vector Sequence List (See Table 2).

ParentSynGTF3C1 is comprises a total of 325 nt. (FIG. 6 B.) The first 125 nt of GTF3C1 intron 34 encompasses the alternative 5′ splice site (1-75 nt) and the canonical 5′ splice site (76-125 nt). This sequence was ligated to the last 200 nt of GTF3C1 intron 34 which encompasses the endogenous 3′ splice site of intron 34. Additional shortened variations of this parent synthetic intron were created as per the Vector Sequence List (See Table 2).

ParentSynARFIP2 comprises a total of 250 nt. FIG. 6C. The first 50 nt of ARFIP2 intron 1 encompasses the 5′ splice site of intron 1 and was ligated to the last 200 nt of ARFIP2 intron 1 which encompasses the endogenous canonical 3′ splice site (1-159 nt) and the alternative 3′ splice site (160-200 nt). Additional shortened variations of this parent sequence synthetic intron were created as per the Vector Sequence List. See Table 2.

ParentSynINTS3 comprises a total of 411 nt. FIG. 6D. All of the INTS3 exon 4 (114 nt) was ligated to 174 nt of INTS3 intron 4. Inserted at position 175 of the synthetic intron was the following sequence: GCCGCCA which encompasses a Kozak sequence (GCCGCC) and an additional adenosine nucleotide. This sequence was followed by the remaining 33 nt of INTS intron 4. 83 nt of INTS3 exon 5 and full-length HSV-TK minus the first 3 nucleotides that code for methionine. The addition of a Kozak sequence functions as a protein translation initiation site and the addition of adenosine allows for a methionine residue to be translated immediately after the Kozak sequence. This allows for synthesis of a transcript with the first 117 nt (coding for 39 amino acid residues) derived from INTS3 intron 4 and exon 5 followed by the entire HSV-TK coding sequence except for the first 3 nucleotides coding for methionine.

ParentSynZNF19 comprises a total of 378 nt. (FIG. 6E.) All of ZNF19 exon 3 (30 nt) was ligated to the first 50 nt of ZNF19 intron 3 which encompasses the endogenous 5′ splice site. This segment was ligated to the last 200 nt of ZNF19 intron 3 encompassing the canonical 3′ splice site. This sequence was then added to the first 55 nt of the ZNF19 alternative splice sequence. The nucleotides GCCATG were added to this sequence to create a Kozak sequence for protein translation initiation followed by 3 nucleotides to code for a methionine residue. The remaining 37 nt of the ZNF19 alternative splice sequence was then added followed by the coding sequence of HSV-TK with the exception of the first adenosine. This allows for the synthesis of a transcript with the first 439 nt (coding for 13 amino acid residues) derived from the ZNF19 3′ alternative splice sequence followed by the entire HSV-TK coding sequence whereby the first amino acid coded for is a valine residue instead of methionine.

TABLE 2
Vector List -

>Lenti_PGK_GFP_P2A_HSV-TK_ARFIP2_150nt<
>Lenti_PGK_GFP_P2A_HSV-TK_ARFIP2_250nt<
>Lenti_PGK_GFP_P2A_HSV-TK_GTF3C1_251nt<
>Lenti_PGK_GFP_P2A_HSV-TK_GTF3C1_250nt_ALT5′SS_MUT<
>Lenti_PGK_GFP_P2A_HSV-TK_GTF3C1_325nt<
>Lenti_PGK_GFP_P2A_HSV-TK_MELK_249nt<
>Lenti_PGK_GFP_P2A_HSV-TK_MELK_397nt<
>Lenti_PGK_GFP_P2A_HSV-TK_MELK_623nt<
>Lenti_PGK_GFP_P2A_HSV-TK-MELK_GTF3C1_356nt_ALT5′SS_MUT<
>MSCV_Ef1a_mCherry_P2A_mEmerald_GTF3C1_251nt<
>MSCV_Ef1a_mCherry_P2A_mEmerald_MELK_249nt<
>MSCV_Ef1a_mCherry_P2A_mEmerald_MELK_623nt<
>MCSV_Ef1a_mCherry_P2A_mEmerald_MELK_GTF3C1_Combo_356nt<
>Lenti_PGK_mCherry_P2A_mEmerald_GTF3C1_250nt<
>Lenti_PGK_mCherry_P2A_mEmerald_MELK_249nt<
>Lenti_PGK_PURO_P2A_HSV-TK_GTF3C1_250nt_ALT5′SS_MUT<
>Lenti_PGK_PURO_P2A_HSV-TK_MELK_249nt<
>Lenti_PGK_PURO_P2A_HSV-TK_MELK_GTF3C1_356nt_ALT5′_MUT<
>pcDNA3.1_CMV_ZNF19_378nt_HSV-TK<
>pcDNA3.1_CMV_INTS3_411nt_HSV-TK<

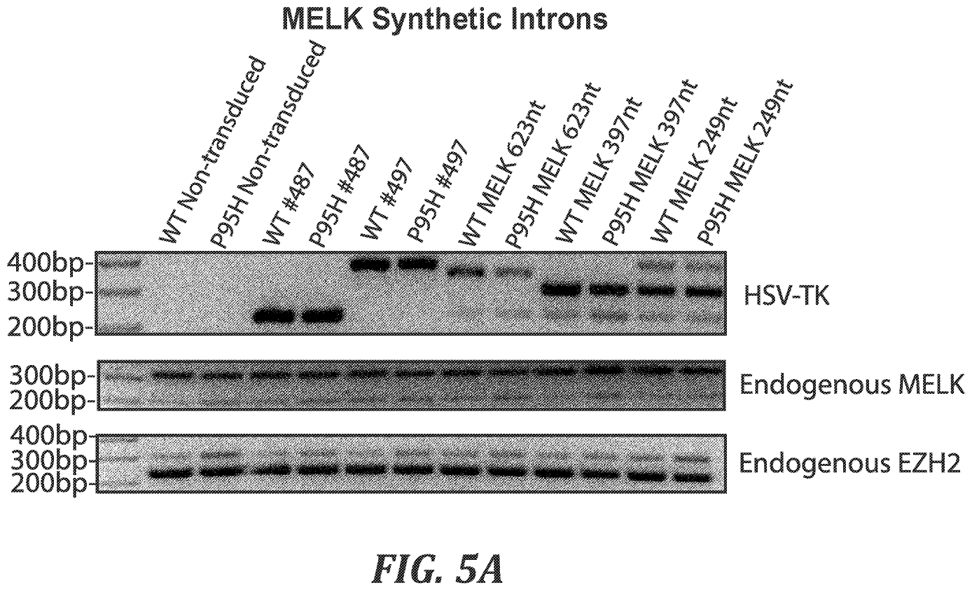

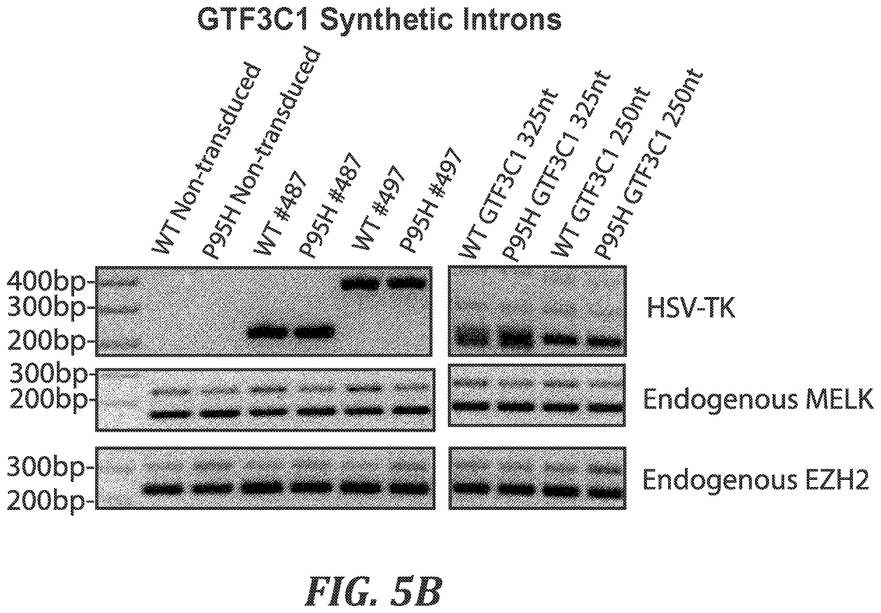

Splicing events for the MELK and GTF3C1 synthetic introns were test in K562 cells. RT-PCR results of splicing events in K562 cells transduced with MELK or GTF3C1 synthetic introns are presented in FIGS. 5A and 5B. WT non-transduced cells were parental SRSF2 wild type K562 cells whereas mutant cells were K562 cells with heterozygous knock-in of the SRSF2 P95H mutation in one allele of the SRSF2 gene (“p95H” cells), P95H Non-transduced, WT #487, P95H #487, WT #497, P95H #497. Gel electrophoresis of RT-PCR products from SRSF2^{+/+} or SRSF2^{+/P95H} isogenic K562 cells amplified using primers for HSV-TK, endogenous MELK or endogenous EZH2 events. Cells utilized include non-transduced cells, cells with construct #487 (positive control), cells with construct #497 (negative control) and cells with GFP/HSV-TK MELK synthetic introns MELK 623nt, MELK 397nt, and MELK 249nt. (FIG. 5A). Gel electrophoresis of RT-PCR products from SRSF2^{+/+} or SRSF2^{+/P95H} isogenic K562 cells amplified using primers for HSV-TK, endogenous MELK or endogenous EZH2 events. Cells utilized in the assays included non-transduced cells, cells with construct #487 (positive control), cells with construct #497 (negative control) and cells with GFP/HSV-TK GTF3C1 synthetic introns GTF3C1_325nt and GTF3C1_250nt.

Next, the therapeutic potential of using synthetic introns to achieve mutation-dependent toxin delivery to cancer cells was tested. The herpes simplex virus thymidine kinase (HSV-TK) system was selected, in which treatment of HSV-TK-expressing cells with the prodrug ganciclovir (GCV) causes cytotoxic metabolite production (Smith, K. O., Galloway, K. S., Kennell, W. L., Ogilvie, K. K. & Radatus, B. K. A new nucleoside analog, 9-[[2-hydroxy-1-(hydroxymethyl) ethoxyl]methyl]guanine, highly active in vitro against herpes simplex virus types 1 and 2. Antimicrob Agents Ch 22, 55-61 (1982), incorporated herein by reference in its entirety), was selected. As GCV is an FDA-approved antiviral therapy with low toxicity for cells lacking HSV-TK, HSV-TK is an attractive system for cancer gene therapy.

As SRSF2 mutations are common across diverse cancer types, synthetic introns may facilitate the development of pan-cancer gene therapies. Furthermore, because synthetic intron function exploits a fundamental property of SRSF2 mutations from which their pro-oncogenic activity arises, resistance to mutation-dependent splicing may be unlikely to develop. Synthetic introns will thereby complement other synthetic biology-based methods for targeted protein expression in response to molecular signals (e.g., Lienert, F., et al. Synthetic biology in mammalian cells: next generation research tools and therapeutics. Nat Rev Mol Cell Bio 15, 95-107 (2014); Wu, M.-R., et al. Engineering advanced cancer therapies with synthetic biology. Nat. Rev. Cancer 19, 187-195 (2019), each of which is incorporated herein by reference in its entirety), including splicing-based devices that utilize RNA aptamers to sense NF-κB and Wnt signaling (Culler, et al. Reprogramming cellular behavior with RNA controllers responsive to endogenous proteins. Science (New York, NY) 330, 1251-1255 (2010), incorporated herein by reference in its entirety) and protease-based devices that sense pro-oncogenic ErbB receptor activity (Chung, H. K. et al. A compact synthetic pathway rewires cancer signaling to therapeutic effector release. Science 364, eaat6982 (2019), incorporated herein by reference in its entirety).

The disclosed synthetic introns are expected to be widely applicable beyond the HSV-TK system. For example, synthetic introns could be used to achieve mutation-dependent expression of other proteins with anti-cancer potential, such as cytokines, chemokines, and cell-surface proteins (Nissim, L. et al. Synthetic RNA-Based Immunomodulatory Gene Circuits for Cancer Immunotherapy. Cell 171, 1138-1150.e15 (2017), incorporated herein by reference in its entirety). Importantly, as synthetic introns yield mutation-dependent splicing and protein expression, delivery of a synthetic intron-bearing therapeutic vector to healthy cells is expected to have negligible consequences. The disclosed synthetic intron-containing fluorescent reporters (FIGS. 1A-1G) could be used to screen for genes and compounds which suppress cancer-specific alterations in RNA splicing. Finally, this study illustrates the power of massively parallel assays for functional interrogation of splicing, including the derivation of rational rules governing mutation-dependent splicing that will facilitate the future design and improvement of these and other synthetic introns.

### Methods

### Work Flow

The workflow for identifying successful locations for inserting a synthetic intron into the coding sequence (CDS) encoding a protein of interest is as follows:

(i) Identify all dinucleotides (two adjacent nucleotides) within the CDS that are identical to the last and first nucleotides of the upstream and downstream exons flanking the endogenous intron from which the synthetic intron is derived.

(ii) For each such dinucleotide, computationally insert the synthetic intron in between the two nucleotides and compute the resulting strengths of the 5′ and 3′ splice sites (e.g., using the MaxEnt algorithm).

(iii) The positions that are most likely to be successful are those which (a) have splice site strengths which are as close as possible to the strengths of the endogenous splice sites, and (b) divide the CDS into two sequences (exons) of roughly equal length.

(iv) If steps 2-3 cannot be achieved, then the CDS can be re-coded by introducing synonymous codon changes as necessary in order to create the desired splice site strengths.

(v) Additional synonymous codon changes can be subsequently introduced to create exonic splicing enhancers (e.g., CCNG, GGNG, CGNG, GCNG, and other sequences that are bound by serine/arginine-rich (SR) proteins or other factors that promote exon recognition) and/or exonic splicing silencers (e.g., TTTGTTCCGT (SEQ ID NO:32), GGGTGGTTTA (SEQ ID NO:33)) and other examples exonic splicing silencers enumerated in Supplemental Table S1 from Wang et al., Cell 119:831-845 (2004); incorporated herein by reference) within the exons in order to alter exon recognition and splicing.

The above procedure can be modified to split a CDS into three or more exons and insert two or more different synthetic introns in between the resulting exons, by iteratively applying the procedure in such a manner as to generate exons of appropriate lengths at the end of the procedure.

This procedure was followed herein to split the CDSs for mEmerald as well as HSV-TK into two exons, separated by a synthetic intron.

The above procedure can also be used to insert one or more synthetic introns into an endogenous or exogenous gene, rather than into a CDS as described above (e.g., to insert a synthetic intron into an endogenous gene in the genome). This gene can already contain zero, one, or more introns.

### Expression Vector Cloning

Several different vector backbones were used for expression and testing the synthetic introns described herein. The vector constructs nomenclature is as follows: >ViralVectorType_Promoter_1^{st}CodingSequence_Linker_2^{nd}CodingSequence_Synthetici ntronName_SyntheticintronNucleotideLength<

### Vector Backbones Utilized:

### 1) Lenti_PGK_GFP_P2A_HSV-TK_Syntheticintron_Nucleotidelength

This nomenclature relates to Lentiviral vectors with a PGK promoter, Green Fluorescent Protein (GFP) as the first coding sequence and selection marker, P2A which serves as a linker between the first and second coding sequence, HSV-TK which serves as the second coding sequence and gene of interest. In these constructs, HSV-TK is interrupted by the synthetic intron of interest. The nucleotide length of each synthetic intron is listed.

This vector was used for the following sequences:
>Lenti_PGK_GFP_P2A_HSV-TK_ARFIP2_150nt
>Lenti_PGK_GFP_P2A_HSV-TK_ARFIP2_250nt
>Lenti_PGK_GFP_P2A_HSV-TK_GTF3C1_250nt
>Lenti_PGK_GFP_P2A_HSV-TK_GTF3C1_250nt_ALT5′SS_MUT
>Lenti_PGK_GFP_P2A_HSV-TK_GTF3C1_325nt
>Lenti_PGK_GFP_P2A_HSV-TK_MELK_249nt
>Lenti_PGK_GFP_P2A_HSV-TK_MELK_397nt
>Lenti_PGK_GFP_P2A_HSV-TK_MELK_623nt
>Lenti_PGK_GFP_P2A_HSV-TK_MELK_GTF3C1_356nt_ALT5′SS_MUT

### 2) MSCV_EF1a_mCherry_P2A_mEmerald_SyntheticIntron_NucleotideLength

This retroviral vector comprises an EF1a promoter, mCherry florescent protein which serves as the 1st coding sequence and selection marker, P2A which severs as a linker between 1st and 2nd coding sequences, and mEmerald florescent protein which serves as the 2nd coding sequence and gene of interest. In these constructs mEmerald is interrupted by the synthetic intron of interest. The nucleotide length of each synthetic intron is listed.

This vector was used for the following sequences:
>MSCV_EF1a_mCherry_P2A_mEmerald_GTF3C1_250nt
>MSCV_EF1a_mCherry_P2A_mEmerald_MELK_249nt
>MSCV_EF1a_mCherry_P2A_mEmerald_MELK_623nt
>MSCV_EF1a_mCherry_P2A_mEmerald_MELK_GTF3C1_Combo_356nt

### 3) Lenti_PGK_mCherry_P2A_mEmerald_SyntheticIntron_NucleotideLength

This Lentiviral vector construct comprises a PGK promoter, mCherry florescent protein which serves as the 1st coding sequence and selection marker, P2A which severs as a linker between 1st and 2nd coding sequences, and mEmerald florescent protein which serves as the 2nd coding sequence and gene of interest. In these constructs mEmerald is interrupted by the synthetic intron of interest. The nucleotide length of each synthetic intron is listed.

This vector was used for the following sequences:
>Lenti_PGK_mCherry_P2A_mEmerald_GTF3C1_250nt
>Lenti_PGK_mCherry_P2A_mEmerald_MELK_249nt

### 4) Lenti_PGK_PURO_P2A_HSV-TK_SyntheticIntron_NucleotideLength

This Lentiviral vector comprises a PGK promoter, Puromycin as the 1st coding sequence and selection marker, P2A which serves as a linker between 1st and 2nd coding sequences, and HSV-TK which serves as the 2nd coding sequence and gene of interest. In these constructs HSV-TK is interrupted by the synthetic intron of interest. The nucleotide length of each synthetic intron is listed.

This vector was used for the following sequences:
>Lenti_PGK_PURO_P2A_HSV-TK_GTF3C1_250nt_ALT5′SS_MUT
>Lenti_PGK_PURO_P2A_HSV-TK_MELK_249nt
>Lenti_PGK_PURO_P2A_HSV-TK_MELK_GTF3C1_356nt_ALT5′SS_MUT
   5) pcDNA3.1_CMV_SyntheticIntronName_NucleotideLength_HSV-TK

The pcDNA3.1 vectors comprise the CMV promoter, and the synthetic intron sequence is inserted upstream of the coding sequence and gene of interest HSV-TK.

This vector was used for the following sequences:
>pcDNA3.1_CMV_ZNF19_378nt_HSV-TK
>pcDNA3.1_CMV_INTS3_411nt_HSV-TK

Table 3 provides the name of the vector construct followed by the nucleotide sequences of the split gene of interest and the synthetic intron.

TABLE 3
Nucleotide sequences for the gene of interest and intron and exon sequences of
the synthetic introns.

1) >Lenti_PGK_GFP_HSV-TK_ARFIP2_150 nt
HSV-TK 5′
atggcttcgtacccctgccatcaacacgcgtctgcgttcgaccaggctgcgcgttctcgcggccatagcaaccgacgtacggcg
ttgcgccctcgccggcagcaagaagccacggaagtccgcctggagcagaaaatgcccacgctactgcgggtttatatagacg
gtcctcacgggatggggaaaaccaccaccacgcaactgctggtggccctgggttcgcgcgacgatatcgtctacgtacccgag
ccgatgacttactggcaggtgctgggggcttccgagacaatcgcgaacatctacaccacacaacaccgcctcgaccagggtga
gatatcggccgg (SEQ ID NO: 5)
ARFIP2 150 nt
gtgagaggaacatgcttgggcgacgggaagttgaacgcacaaacctgtccgaggcctccttcttctagagtgctggccagtgtc
cagaacttctgtgttgggctttgcagggtgctggggtggagagttttactccaatacctttcctag (SEQ ID NO: 9)
HSV-TK 3′
ggacgcggcggtggtaatgacaagcgcccagataacaatgggcatgccttatgccgtgaccgacgccgttctggctcctcatat
cgggggggaggctgggagctcacatgccccgcccccggccctcaccctcatcttcgaccgccatcccatcgccgccctcctgt
gctacccggccgcgcgataccttatgggcagcatgaccccccaggccgtgctggcgttcgtggccctcatcccgccgaccttg
cccggcacaaacatcgtgttgggggcccttccggaggacagacacatcgaccgcctggccaaacgccagcgccccggcgag
cggcttgacctggctatgctggccgcgattcgccgcgtttacgggctgcttgccaatacggtgcggtatctgcagggcggcggg
tcgtggcgggaggattggggacagctttcggggacggccgtgccgccccagggtgccgagccccagagcaacgcgggccc
acgaccccatatcggggacacgttatttaccctgtttcgggcccccgagttgctggcccccaacggcgacctgtacaacgtgttt
gcctgggccttggacgtcttggccaaacgcctccgtcccatgcacgtctttatcctggattacgaccaatcgcccgccggctgcc
gggacgccctgctgcaacttacctccgggatggtccagacccacgtcaccacccccggctccataccgacgatctgcgacctg
gcgcgcacgtttgcccgggagatgggggaggctaactga (SEQ ID NO: 6)
2) >Lenti_PGK_GFP_P2A_HSV-TK_ARFIP2_250 nt
HSV-TK 5′
atggcttcgtacccctgccatcaacacgcgtctgcgttcgaccaggctgcgcgttctcgcggccatagcaaccgacgtacggcg
ttgcgccctcgccggcagcaagaagccacggaagtccgcctggagcagaaaatgcccacgctactgcgggtttatatagacg
gtcctcacgggatggggaaaaccaccaccacgcaactgctggtggccctgggttcgcgcgacgatatcgtctacgtacccgag
ccgatgacttactggcaggtgctgggggcttccgagacaatcgcgaacatctacaccacacaacaccgcctcgaccagggtga
gatatcggccgg (SEQ ID NO: 5)
ARFIP2 250 nt
gtgagaggaacatgcttgggcgacgggaagttgaacgcacaaacctgtcctgcgttttggaggggagaaatacatatatctgaa
gaaaggaatgatggccaaacgctgggacactcaaaccctgggacagcctttaaagaaatgacagaggaggcctccttcttctag
agtgctggccagtgtccagaacttctgtgttgggctttgcagggtgctggggtggagagttttactccaatacctttcctag
(SEQ ID NO: 10)
HSV-TK 3′
ggacgcggcggtggtaatgacaagcgcccagataacaatgggcatgccttatgccgtgaccgacgccgttctggctcctcatat
cgggggggaggctgggagctcacatgccccgcccccggccctcaccctcatcttcgaccgccatcccatcgccgccctcctgt
gctacccggccgcgcgataccttatgggcagcatgaccccccaggccgtgctggcgttcgtggccctcatcccgccgaccttg
cccggcacaaacatcgtgttgggggcccttccggaggacagacacatcgaccgcctggccaaacgccagcgccccggcgag
cggcttgacctggctatgctggccgcgattcgccgcgtttacgggctgcttgccaatacggtgcggtatctgcagggcggcggg
tcgtggcgggaggattggggacagctttcggggacggccgtgccgccccagggtgccgagccccagagcaacgcgggccc
acgaccccatatcggggacacgttatttaccctgtttcgggcccccgagttgctggcccccaacggcgacctgtacaacgtgttt
gcctgggccttggacgtcttggccaaacgcctccgtcccatgcacgtctttatcctggattacgaccaatcgcccgccggctgcc
gggacgccctgctgcaacttacctccgggatggtccagacccacgtcaccacccccggctccataccgacgatctgcgacctg
gcgcgcacgtttgcccgggagatgggggaggctaactga (SEQ ID NO: 6)
3) >Lenti_PGK_GFP_P2A_HSV-TK_GTF3C1_250 nt<
HSV-TK 5′
atggcttcgtacccctgccatcaacacgcgtctgcgttcgaccaggctgcgcgttctcgcggccatagcaaccgacgtacggcg
ttgcgccctcgccggcagcaagaagccacggaagtccgcctggagcagaaaatgcccacgctactgcgggtttatatagacg
gtcctcacgggatggggaaaaccaccaccacgcaactgctggtggccctgggttcgcgcgacgatatcgtctacgtacccgag
ccgatgacttactggcaggtgctgggggcttccgagacaatcgcgaacatctacaccacacaacaccgcctcgaccagggtga
gatatcggccgg (SEQ ID NO: 5)
GTF3C1 250 nt
gtgagttcagttccccaggccaagagcagctgagcggccaggcgcagcctccagagggctctgaagaccccagaggtaccg
cgcgccttgtcccccaccccacctcaccccaccctggctttccctttgacacttcatcccctgagcctcgaggtgccctgctcatg
ctaggtaccatgctgaaggttggagggcagagatggcttccctgactttccgagtctctcacattctcttcctcttccctcag
(SEQ ID NO: 11)
HSV-TK 3′
ggacgcggcggtggtaatgacaagcgcccagataacaatgggcatgccttatgccgtgaccgacgccgttctggctcctcatat
cgggggggaggctgggagctcacatgccccgcccccggccctcaccctcatcttcgaccgccatcccatcgccgccctcctgt
gctacccggccgcgcgataccttatgggcagcatgaccccccaggccgtgctggcgttcgtggccctcatcccgccgaccttg
cccggcacaaacatcgtgttgggggcccttccggaggacagacacatcgaccgcctggccaaacgccagcgccccggcgag
cggcttgacctggctatgctggccgcgattcgccgcgtttacgggctgcttgccaatacggtgcggtatctgcagggcggcggg
tcgtggcgggaggattggggacagctttcggggacggccgtgccgccccagggtgccgagccccagagcaacgcgggccc
acgaccccatatcggggacacgttatttaccctgtttcgggcccccgagttgctggcccccaacggcgacctgtacaacgtgttt
gcctgggccttggacgtcttggccaaacgcctccgtcccatgcacgtctttatcctggattacgaccaatcgcccgccggctgcc
gggacgccctgctgcaacttacctccgggatggtccagacccacgtcaccacccccggctccataccgacgatctgcgacctg
gcgcgcacgtttgcccgggagatgggggaggctaactga (SEQ ID NO: 6)
4) >Lenti_PGK_GFP_P2A_HSV-TK_GTF3C1_250 nt_ALT5′SS_MUT<
HSV-TK 5′ with alt5′ss mutation T > G at position 330 of HSV-TK
atggcttcgtacccctgccatcaacacgcgtctgcgttcgaccaggctgcgcgttctcgcggccatagcaaccgacgtacggcg
ttgcgccctcgccggcagcaagaagccacggaagtccgcctggagcagaaaatgcccacgctactgcgggtttatatagacg
gtcctcacgggatggggaaaaccaccaccacgcaactgctggtggccctgggttcgcgcgacgatatcgtctacgtacccgag
ccgatgacttactggcaggtgctgggggcttccgagacaatcgcgaacatctacaccacacaacaccgcctcgaccaggggg
agatatcggccgg (SEQ ID NO: 5)
GTF3C1 250 nt
gtgagttcagttccccaggccaagagcagctgagcggccaggcgcagcctccagagggctctgaagaccccagaggtaccg
cgcgccttgtcccccaccccacctcaccccaccctggctttccctttgacacttcatcccctgagcctcgaggtgccctgctcatg
ctaggtaccatgctgaaggttggagggcagagatggcttccctgactttccgagtctctcacattctcttcctcttccctcag
(SEQ ID NO: 10)
HSV-TK 3′
ggacgcggcggtggtaatgacaagcgcccagataacaatgggcatgccttatgccgtgaccgacgccgttctggctcctcatat
cgggggggaggctgggagctcacatgccccgcccccggccctcaccctcatcttcgaccgccatcccatcgccgccctcctgt
gctacccggccgcgcgataccttatgggcagcatgaccccccaggccgtgctggcgttcgtggccctcatcccgccgaccttg
cccggcacaaacatcgtgttgggggcccttccggaggacagacacatcgaccgcctggccaaacgccagcgccccggcgag
cggcttgacctggctatgctggccgcgattcgccgcgtttacgggctgcttgccaatacggtgcggtatctgcagggcggcggg
tcgtggcgggaggattggggacagctttcggggacggccgtgccgccccagggtgccgagccccagagcaacgcgggccc
acgaccccatatcggggacacgttatttaccctgtttcgggcccccgagttgctggcccccaacggcgacctgtacaacgtgttt
gcctgggccttggacgtcttggccaaacgcctccgtcccatgcacgtctttatcctggattacgaccaatcgcccgccggctgcc
gggacgccctgctgcaacttacctccgggatggtccagacccacgtcaccacccccggctccataccgacgatctgcgacctg
gcgcgcacgtttgcccgggagatgggggaggctaactga (SEQ ID NO: 6)
5) >Lenti_PGK_PURO_P2A_HSV-TK_GTF3C1_250 nt_ALT5′SS_MUT<
HSV-TK 5′ with alt5′ss mutation T > G at position 330 of HSV-TK
atggcttcgtacccctgccatcaacacgcgtctgcgttcgaccaggctgcgcgttctcgcggccatagcaaccgacgtacggcg
ttgcgccctcgccggcagcaagaagccacggaagtccgcctggagcagaaaatgcccacgctactgcgggtttatatagacg
gtcctcacgggatggggaaaaccaccaccacgcaactgctggtggccctgggttcgcgcgacgatatcgtctacgtacccgag
ccgatgacttactggcaggtgctgggggcttccgagacaatcgcgaacatctacaccacacaacaccgcctcgaccaggggg
agatatcggccgg (SEQ ID NO: 19)
GTF3C1 250 nt
gtgagttcagttccccaggccaagagcagctgagcggccaggcgcagcctccagagggctctgaagaccccagaggtaccg
cgcgccttgtcccccaccccacctcaccccaccctggctttccctttgacacttcatcccctgagcctcgaggtgccctgctcatg
ctaggtaccatgctgaaggttggagggcagagatggcttccctgactttccgagtctctcacattctcttcctcttccctcag
(SEQ ID NO: 11)
HSV-TK 3′
ggacgcggcggtggtaatgacaagcgcccagataacaatgggcatgccttatgccgtgaccgacgccgttctggctcctcatat
cgggggggaggctgggagctcacatgccccgcccccggccctcaccctcatcttcgaccgccatcccatcgccgccctcctgt
gctacccggccgcgcgataccttatgggcagcatgaccccccaggccgtgctggcgttcgtggccctcatcccgccgaccttg
cccggcacaaacatcgtgttgggggcccttccggaggacagacacatcgaccgcctggccaaacgccagcgccccggcgag
cggcttgacctggctatgctggccgcgattcgccgcgtttacgggctgcttgccaatacggtgcggtatctgcagggcggcggg
tcgtggcgggaggattggggacagctttcggggacggccgtgccgccccagggtgccgagccccagagcaacgcgggccc
acgaccccatatcggggacacgttatttaccctgtttcgggcccccgagttgctggcccccaacggcgacctgtacaacgtgttt
gcctgggccttggacgtcttggccaaacgcctccgtcccatgcacgtctttatcctggattacgaccaatcgcccgccggctgcc
gggacgccctgctgcaacttacctccgggatggtccagacccacgtcaccacccccggctccataccgacgatctgcgacctg
gcgcgcacgtttgcccgggagatgggggaggctaactga (SEQ ID NO: 6)
6) >Lenti_PGK_mCherry_P2A_mEmerald_GTF3C1_250 nt<
mEmerald 5′
atggtgagcaagggcgaggagctgttcaccggggtggtgcccatcctggtcgagctggacggcgacgtaaacggccacaagt
tcagcgtgtccggcgagggcgagggcgatgccacctacggcaagctgaccctgaagttcatctgcaccaccggcaagctgcc
cgtgccctggcccaccctcgtgaccaccttgacctacggcgtgcagtgcttcgcccgctaccccgaccacatgaagcagcacg
acttcttcaagtccgccatgcccgaaggctacgtccaggagcgcaccatcttcttcaaggacgacggcaactacaagacccgcg
ccgaggtgaagttcgag (SEQ ID NO: 7)
GTF3C1 250 nt
gtgagttcagttccccaggccaagagcagctgagcggccaggcgcagcctccagagggctctgaagaccccagaggtaccg
cgcgccttgtcccccaccccacctcaccccaccctggctttccctttgacacttcatcccctgagcctcgaggtgccctgctcatg
ctaggtaccatgctgaaggttggagggcagagatggcttccctgactttccgagtctctcacattctcttcctcttccctcag
(SEQ ID NO: 11)
mEmerald 3′
ggcgacaccctggtgaaccgcatcgagctgaagggcatcgacttcaaggaggacggcaacatcctggggcacaagctggag
tacaactacaacagccacaaggtctatatcaccgccgacaagcagaagaacggcatcaaggtgaacttcaagacccgccacaa
catcgaggacggcagcgtgcagctcgccgaccactaccagcagaacacccccatcggcgacggccccgtgctgctgcccga
caaccactacctgagcacccagtccaagctgagcaaagaccccaacgagaagcgcgatcacatggtcctgctggagttcgtga
ccgccgccgggatcactctcggcatggacgagctgtacaagtaa (SEQ ID NO: 8)
7) >MSCV_EF1a_mCherry_P2A_mEmerald_GTF3C1_250 nt<
mEmerald 5′
atggtgagcaagggcgaggagctgttcaccggggtggtgcccatcctggtcgagctggacggcgacgtaaacggccacaagt
tcagcgtgtccggcgagggcgagggcgatgccacctacggcaagctgaccctgaagttcatctgcaccaccggcaagctgcc
cgtgccctggcccaccctcgtgaccaccttgacctacggcgtgcagtgcttcgcccgctaccccgaccacatgaagcagcacg
acttcttcaagtccgccatgcccgaaggctacgtccaggagcgcaccatcttcttcaaggacgacggcaactacaagacccgcg
ccgaggtgaagttcgag (SEQ ID NO: 7)
GTF3C1 250 nt
gtgagttcagttccccaggccaagagcagctgagcggccaggcgcagcctccagagggctctgaagaccccagaggtaccg
cgcgccttgtcccccaccccacctcaccccaccctggctttccctttgacacttcatcccctgagcctcgaggtgccctgctcatg
ctaggtaccatgctgaaggttggagggcagagatggcttccctgactttccgagtctctcacattctcttcctcttccctcag
(SEQ ID NO: 11)
mEmerald 3′
ggcgacaccctggtgaaccgcatcgagctgaagggcatcgacttcaaggaggacggcaacatcctggggcacaagctggag
tacaactacaacagccacaaggtctatatcaccgccgacaagcagaagaacggcatcaaggtgaacttcaagacccgccacaa
catcgaggacggcagcgtgcagctcgccgaccactaccagcagaacacccccatcggcgacggccccgtgctgctgcccga
caaccactacctgagcacccagtccaagctgagcaaagaccccaacgagaagcgcgatcacatggtcctgctggagttcgtga
ccgccgccgggatcactctcggcatggacgagctgtacaagtaa (SEQ ID NO: 8)
8) >Lenti_PGK_GFP_P2A_HSV-TK_GTF3C1_325 nt<
HSV-TK 5′
atggcttcgtacccctgccatcaacacgcgtctgcgttcgaccaggctgcgcgttctcgcggccatagcaaccgacgtacggcg
ttgcgccctcgccggcagcaagaagccacggaagtccgcctggagcagaaaatgcccacgctactgcgggtttatatagacg
gtcctcacgggatggggaaaaccaccaccacgcaactgctggtggccctgggttcgcgcgacgatatcgtctacgtacccgag
ccgatgacttactggcaggtgctgggggcttccgagacaatcgcgaacatctacaccacacaacaccgcctcgaccagggtga
gatatcggccgg (SEQ ID NO: 5)
GTF3C1 325 nt
gtgagttcagttccccaggccaagagcagctgagcggccaggcgcagcctccagagggctctgaagaccccagaggtaccg
cgcgccttgtcccccaccccacctcaccccaccctggctttccccgctgtgtgcggcagtctgcccggccctgccacctccagg
gtggccgctctgtgagatgctcattgtcctttttttttgacacttcatcccctgagcctcgaggtgccctgctcatgctaggtaccatg
ctgaaggttggagggcagagatggcttccctgactttccgagtctctcacattctcttcctcttccctcag (SEQ ID NO: 12)
HSV-TK 3′
ggacgcggcggtggtaatgacaagcgcccagataacaatgggcatgccttatgccgtgaccgacgccgttctggctcctcatat
cgggggggaggctgggagctcacatgccccgcccccggccctcaccctcatcttcgaccgccatcccatcgccgccctcctgt
gctacccggccgcgcgataccttatgggcagcatgaccccccaggccgtgctggcgttcgtggccctcatcccgccgaccttg
cccggcacaaacatcgtgttgggggcccttccggaggacagacacatcgaccgcctggccaaacgccagcgccccggcgag
cggcttgacctggctatgctggccgcgattcgccgcgtttacgggctgcttgccaatacggtgcggtatctgcagggcggcggg
tcgtggcgggaggattggggacagctttcggggacggccgtgccgccccagggtgccgagccccagagcaacgcgggccc
acgaccccatatcggggacacgttatttaccctgtttcgggcccccgagttgctggcccccaacggcgacctgtacaacgtgttt
gcctgggccttggacgtcttggccaaacgcctccgtcccatgcacgtctttatcctggattacgaccaatcgcccgccggctgcc
gggacgccctgctgcaacttacctccgggatggtccagacccacgtcaccacccccggctccataccgacgatctgcgacctg
gcgcgcacgtttgcccgggagatgggggaggctaactga (SEQ ID NO: 6)
9) >Lenti_PGK_GFP_P2A_HSV-TK_MELK_249 nt<
HSV-TK 5′
atggcttcgtacccctgccatcaacacgcgtctgcgttcgaccaggctgcgcgttctcgcggccatagcaaccgacgtacggcg
ttgcgccctcgccggcagcaagaagccacggaagtccgcctggagcagaaaatgcccacgctactgcgggtttatatagacg
gtcctcacgggatggggaaaaccaccaccacgcaactgctggtggccctgggttcgcgcgacgatatcgtctacgtacccgag
ccgatgacttactggcaggtgctgggggcttccgagacaatcgcgaacatctacaccacacaacaccgcctcgaccagggtga
gatatcggccgg (SEQ ID NO: 5)
MELK 249 nt
gtaagtgttactgcctgtgcgtgactgtattttaaatattgaatcatagtactgtcatctttaagttcttctgtctttcattgagtagtcaaat
aattggagtctggaattggtgtgaagatgatttatcaacaggtgctgctactccccgaacatcacaggttcgtcattcttattactcaa
ggaatgttgtaaagaaattgacttttcttcttcagtgtgctttatctagtaactttttttttccag (SEQ ID NO: 13)
HSV-TK 3′
ggacgcggcggtggtaatgacaagcgcccagataacaatgggcatgccttatgccgtgaccgacgccgttctggctcctcatat
cgggggggaggctgggagctcacatgccccgcccccggccctcaccctcatccttcgaccgccatcccatcgccgccctcctgt
gctacccggccgcgcgataccttatgggcagcatgaccccccaggccgtgctggcgttcgtggccctcatcccgccgaccttg
cccggcacaaacatcgtgttgggggcccttccggaggacagacacatcgaccgcctggccaaacgccagcgccccggcgag
cggcttgacctggctatgctggccgcgattcgccgcgtttacgggctgcttgccaatacggtgcggtatctgcagggcggcggg
tcgtggcgggaggattggggacagctttcggggacggccgtgccgccccagggtgccgagccccagagcaacgcgggccc
acgaccccatatcggggacacgttatttaccctgtttcgggcccccgagttgctggcccccaacggcgacctgtacaacgtgttt
gcctgggccttggacgtcttggccaaacgcctccgtcccatgcacgtctttatcctggattacgaccaatcgcccgccggctgcc
gggacgccctgctgcaacttacctccgggatggtccagacccacgtcaccacccccggctccataccgacgatctgcgacctg
gcgcgcacgtttgcccgggagatgggggaggctaactga (SEQ ID NO: 6)
10) >Lenti_PGK_mCherry_P2A_mEmerald_MELK_249 nt<
mEmerald 5′
atggtgagcaagggcgaggagctgttcaccggggtggtgcccatcctggtcgagctggacggcgacgtaaacggccacaagt
tcagcgtgtccggcgagggcgagggcgatgccacctacggcaagctgaccctgaagttcatctgcaccaccggcaagctgcc
cgtgccctggcccaccctcgtgaccaccttgacctacggcgtgcagtgcttcgcccgctaccccgaccacatgaagcagcacg
acttcttcaagtccgccatgcccgaaggctacgtccaggagcgcaccatcttcttcaaggacgacggcaactacaagacccgcg
ccgaggtgaagttcgag (SEQ ID NO: 7)
MELK 249 nt Minigene (Intron 12, Exon 13, Intron 13)
gtaagtgttactgcctgtgcgtgactgtattttaaatattgaatcatagtactgtcatctttaagttcttctgtctttcattgagtagtcaaat
aattggagtctggaattggtgtgaagatgatttatcaacaggtgctgctactccccgaacatcacaggttcgtcattcttattactcaa
ggaatgttgtaaagaaattgacttttcttcttcagtgtgctttatctagtaactttttttttccag (SEQ ID NO: 13)
mEmerald 3′
ggcgacaccctggtgaaccgcatcgagctgaagggcatcgacttcaaggaggacggcaacatcctggggcacaagctggag
tacaactacaacagccacaaggtctatatcaccgccgacaagcagaagaacggcatcaaggtgaacttcaagacccgccacaa
catcgaggacggcagcgtgcagctcgccgaccactaccagcagaacacccccatcggcgacggccccgtgctgctgcccga
caaccactacctgagcacccagtccaagctgagcaaagaccccaacgagaagcgcgatcacatggtcctgctggagttcgtga
ccgccgccgggatcactctcggcatggacgagctgta (SEQ ID NO: 8)
11) >MSCV_EF1a_mCherry_P2A_mEmerald_MELK_249 nt<
mEmerald 5′
atggtgagcaagggcgaggagctgttcaccggggtggtgcccatcctggtcgagctggacggcgacgtaaacggccacaagt
tcagcgtgtccggcgagggcgagggcgatgccacctacggcaagctgaccctgaagttcatctgcaccaccggcaagctgcc
cgtgccctggcccaccctcgtgaccaccttgacctacggcgtgcagtgcttcgcccgctaccccgaccacatgaagcagcacg
acttcttcaagtccgccatgcccgaaggctacgtccaggagcgcaccatcttcttcaaggacgacggcaactacaagacccgcg
ccgaggtgaagttcgag (SEQ ID NO: 7)
MELK 249 nt Minigene (Intron 12, Exon 13, Intron 13)
gtaagtgttactgcctgtgcgtgactgtattttaaatattgaatcatagtactgtcatctttaagttcttctgtctttcattgagtagtcaaat
aattggagtctggaattggtgtgaagatgatttatcaacaggtgctgctactccccgaacatcacaggttcgtcattcttattactcaa
ggaatgttgtaaagaaattgacttttcttcttcagtgtgctttatctagtaactttttttttccag (SEQ ID NO: 13)
3′Emerald
ggcgacaccctggtgaaccgcatcgagctgaagggcatcgacttcaaggaggacggcaacatcctggggcacaagctggag
tacaactacaacagccacaaggtctatatcaccgccgacaagcagaagaacggcatcaaggtgaacttcaagacccgccacaa
catcgaggacggcagcgtgcagctcgccgaccactaccagcagaacacccccatcggcgacggccccgtgctgctgcccga
caaccactacctgagcacccagtccaagctgagcaaagaccccaacgagaagcgcgatcacatggtcctgctggagttcgtga
ccgccgccgggatcactctcggcatggacgagctgtacaagtaa (SEQ ID NO: 8)
12) >Lenti_PGK_PURO_P2A_HSV-TK_MELK_249 nt<
HSV-TK 5′
atggcttcgtacccctgccatcaacacgcgtctgcgttcgaccaggctgcgcgttctcgcggccatagcaaccgacgtacggcg
ttgcgccctcgccggcagcaagaagccacggaagtccgcctggagcagaaaatgcccacgctactgcgggtttatatagacg
gtcctcacgggatggggaaaaccaccaccacgcaactgctggtggccctgggttcgcgcgacgatatcgtctacgtacccgag
ccgatgacttactggcaggtgctgggggcttccgagacaatcgcgaacatctacaccacacaacaccgcctcgaccagggtga
gatatcggccgg (SEQ ID NO: 5)
MELK 249 nt
gtaagtgttactgcctgtgcgtgactgtattttaaatattgaatcatagtactgtcatctttaagttcttctgtctttcattgagtagtcaaat
aattggagtctggaattggtgtgaagatgatttatcaacaggtgctgctactccccgaacatcacaggttcgtcattcttattactcaa
ggaatgttgtaaagaaattgacttttcttcttcagtgtgctttatctagtaactttttttttccag (SEQ ID NO: 13)
3′ HSV-TK
ggacgcggcggtggtaatgacaagcgcccagataacaatgggcatgccttatgccgtgaccgacgccgttctggctcctcatat
cgggggggaggctgggagctcacatgccccgcccccggccctcaccctcatcttcgaccgccatcccatcgccgccctcctgt
gctacccggccgcgcgataccttatgggcagcatgaccccccaggccgtgctggcgttcgtggccctcatcccgccgaccttg
cccggcacaaacatcgtgttgggggcccttccggaggacagacacatcgaccgcctggccaaacgccagcgccccggcgag
cggcttgacctggctatgctggccgcgattcgccgcgtttacgggctgcttgccaatacggtgcggtatctgcagggcggcggg
tcgtggcgggaggattggggacagctttcggggacggccgtgccgccccagggtgccgagccccagagcaacgcgggccc
acgaccccatatcggggacacgttatttaccctgtttcgggcccccgagttgctggcccccaacggcgacctgtacaacgtgttt
gcctgggccttggacgtcttggccaaacgcctccgtcccatgcacgtctttatcctggattacgaccaatcgcccgccggctgcc
gggacgccctgctgcaacttacctccgggatggtccagacccacgtcaccacccccggctccataccgacgatctgcgacctg
gcgcgcacgtttgcccgggagatgggggaggctaactga (SEQ ID NO: 6)
13) >Lenti_PGK_GFP_P2A_HSV-TK_MELK_397 nt<
HSV-TK 5′
atggcttcgtacccctgccatcaacacgcgtctgcgttcgaccaggctgcgcgttctcgcggccatagcaaccgacgtacggcg
ttgcgccctcgccggcagcaagaagccacggaagtccgcctggagcagaaaatgcccacgctactgcgggtttatatagacg
gtcctcacgggatggggaaaaccaccaccacgcaactgctggtggccctgggttcgcgcgacgatatcgtctacgtacccgag
ccgatgacttactggcaggtgctgggggcttccgagacaatcgcgaacatctacaccacacaacaccgcctcgaccagggtga
gatatcggccgg (SEQ ID NO: 5)
MELK 397 nt
gtaagtgttactgcctgttgtgtcttaagtacattttatgatgttcacacagtgataaaattgcctaatacatttctcagaatgtgtctctg
tcgttaagtgacgcgtgactgtattttaaatattgaatcatagtactgtcatctttaagttcttctgtctttcattgagtagtcaaataattg
gagtctggaattggtgtgaagatgatttatcaacaggtgctgctactccccgaacatcacaggttcgtcattcttattactatttaaata
tccttttgataattcattttaataaaaatagtacaaggtagtttgcaaatgatcaaggaatgttgtaaagaaattgacttttcttcttcagt
gtgctttatctagtaactttttttttccag (SEQ ID NO: 14)
HSV-TK 3′
ggacgcggcggtggtaatgacaagcgcccagataacaatgggcatgccttatgccgtgaccgacgccgttctggctcctcatat
ggggggaggctgggagctcacatgccccgcccccggccctcaccctcatcttcgaccgccatcccatcgccgccctcctgt
gctacccggccgcgcgataccttatgggcagcatgaccccccaggccgtgctggcgttcgtggccctcatcccgccgaccttg
cccggcacaaacatcgtgttgggggcccttccggaggacagacacatcgaccgcctggccaaacgccagcgccccggcgag
cggcttgacctggctatgctggccgcgattcgccgcgtttacgggctgcttgccaatacggtgcggtatctgcagggcggcggg
tcgtggcgggaggattggggacagctttcggggacggccgtgccgccccagggtgccgagccccagagcaacgcgggccc
acgaccccatatcggggacacgttatttaccctgtttcgggcccccgagttgctggcccccaacggcgacctgtacaacgtgttt
gcctgggccttggacgtcttggccaaacgcctccgtcccatgcacgtctttatcctggattacgaccaatcgcccgccggctgcc
gggacgccctgctgcaacttacctccgggatggtccagacccacgtcaccacccccggctccataccgacgatctgcgacctg
gcgcgcacgtttgcccgggagatgggggaggctaactga (SEQ ID NO: 6)
14) >Lenti_PGK_GFP_P2A_HSV-TK_MELK_623 nt<
HSV-TK 5′
atggcttcgtacccctgccatcaacacgcgtctgcgttcgaccaggctgcgcgttctcgcggccatagcaaccgacgtacggcg
ttgcgccctcgccggcagcaagaagccacggaagtccgcctggagcagaaaatgcccacgctactgcgggtttatatagacg
gtcctcacgggatggggaaaaccaccaccacgcaactgctggtggccctgggttcgcgcgacgatatcgtctacgtacccgag
ccgatgacttactggcaggtgctgggggcttccgagacaatcgcgaacatctacaccacacaacaccgcctcgaccagggtga
gatatcggccgg (SEQ ID NO: 5)
MELK 623 nt
gtaagtgttactgcctgttgtgtcttgccacgtgccctccctgttcctgacagcctaggtgtggcctaggtgtgtagtaggctctgac
gtctcagtttgtgtaagtacattttatgatgttcacacagtgataaaattgcctaatacatttctcagaatgtgtctctgtcgttaagtgac
gcgtgactgtattttaaatattgaatcatagtactgtcatctttaagttcttctgtctttcattgagtagtcaaataattggagtctggaag
atgtgaccgcaagtgataaaaattatgtggcgggattaatagactatgattggtgtgaagatgatttatcaacaggtgctgctactcc
ccgaacatcacaggttcgtcattcttattactatttaaggcagaatctattgtttcaattatatgtcaatagaacagactaaagtgatcta
catgtattaaagaaaactatatattttcaaatttcctaatgtcaataatatccttttgataattcattttaataaaaatagtacaaggtagttt
gcaaatgatcaaggaatgttgtaaagaaattgacttttcttcttcagtgtgctttatctagtaactttttttttccag
(SEQ ID NO: 15)
HSV-TK 3′
ggacgcggcggtggtaatgacaagcgcccagataacaatgggcatgccttatgccgtgaccgacgccgttctggctcctcatat
cgggggggaggctgggagctcacatgccccgcccccggccctcaccctcatcttcgaccgccatcccatcgccgccctcctgt
gctacccggccgcgcgataccttatgggcagcatgaccccccaggccgtgctggcgttcgtggccctcatcccgccgaccttg
cccggcacaaacatcgtgttgggggcccttccggaggacagacacatcgaccgcctggccaaacgccagcgccccggcgag
cggcttgacctggctatgctggccgcgattcgccgcgtttacgggctgcttgccaatacggtgcggtatctgcagggcggcggg
tcgtggcgggaggattggggacagctttcggggacggccgtgccgccccagggtgccgagccccagagcaacgcgggccc
acgaccccatatcggggacacgttatttaccctgtttcgggcccccgagttgctggcccccaacggcgacctgtacaacgtgttt
gcctgggccttggacgtcttggccaaacgcctccgtcccatgcacgtctttatcctggattacgaccaatcgcccgccggctgcc
gggacgccctgctgcaacttacctccgggatggtccagacccacgtcaccacccccggctccataccgacgatctgcgacctg
gcgcgcacgtttgcccgggagatgggggaggctaactga (SEQ ID NO: 6)
15) >MSCV_EF1a_mCherry_P2A_mEmerald_MELK_623 nt<
mEmerald 5′
atggtgagcaagggcgaggagctgttcaccggggggtgcccatcctggtcgagctggacggcgacgtaaacggccacaagt
tcagcgtgtccggcgagggcgagggcgatgccacctacggcaagctgaccctgaagttcatctgcaccaccggcaagctgcc
cgtgccctggcccaccctcgtgaccaccttgacctacggcgtgcagtgcttcgcccgctaccccgaccacatgaagcagcacg
acttcttcaagtccgccatgcccgaaggctacgtccaggagcgcaccatcttcttcaaggacgacggcaactacaagacccgcg
ccgaggtgaagttcgag (SEQ ID NO: 7)
MELK 623 nt
gtaagtgttactgcctgttgtgtcttgccacgtgccctccctgttcctgacagcctaggtgtggcctaggtgtgtagtaggctctgac
gtctcagtttgtgtaagtacattttatgatgttcacacagtgataaaattgcctaatacatttctcagaatgtgtctctgtcgttaagtgac
gcgtgactgtattttaaatattgaatcatagtactgtcatctttaagttcttctgtctttcattgagtagtcaaataattggagtctggaag
atgtgaccgcaagtgataaaaattatgtggcgggattaatagactatgattggtgtgaagatgatttatcaacaggtgctgctactcc
ccgaacatcacaggttcgtcattcttattactatttaaggcagaatctattgtttcaattatatgtcaatagaacagactaaagtgatcta
catgtattaaagaaaactatatattttcaaatttcctaatgtcaataatatccttttgataattcattttaataaaaatagtacaaggtagttt
gcaaatgatcaaggaatgttgtaaagaaattgacttttcttcttcagtgtgctttatctagtaactttttttttccag
(SEQ ID NO: 15)
mEmerald 3′
ggcgacaccctggtgaaccgcatcgagctgaagggcatcgacttcaaggaggacggcaacatcctggggcacaagctggag
tacaactacaacagccacaaggtctatatcaccgccgacaagcagaagaacggcatcaaggtgaacttcaagacccgccacaa
catcgaggacggcagcgtgcagctcgccgaccactaccagcagaacacccccatcggcgacggccccgtgctgctgcccga
caaccactacctgagcacccagtccaagctgagcaaagaccccaacgagaagcgcgatcacatggtcctgctggagttcgtga
ccgccgccgggatcactctcggcatggacgagctgtacaagtaa (SEQ ID NO: 8)
16) >Lenti_PGK_GFP_P2A_HSV-TK_MELK_GTF3C1_356 nt_ALT5′SS_MUT<
HSV-TK 5′ with alt5′ss mutation
atggcttcgtacccctgccatcaacacgcgtctgcgttcgaccaggctgcgcgttctcgcggccatagcaaccgacgtacggcg
ttgcgccctcgccggcagcaagaagccacggaagtccgcctggagcagaaaatgcccacgctactgcgggtttatatagacg
gtcctcacgggatggggaaaaccaccaccacgcaactgctggtggccctgggttcgcgcgacgatatcgtctacgtacccgag
ccgatgacttactggcaggtgctgggggcttccgagacaatcgcgaacatctacaccacacaacaccgcctcgaccaggggg
agatatcggccgg (SEQ ID NO: 19)
MELK/GTF3C1 Combo 356 nt
gtgagttcagttccccaggccaagagcagctgagcggccaggcgcagcctccagagggctctgaagaccccagaggtaccg
cgcgccttgtcccccaccccacctcaccccaccctggctttcccgcgtgactgtattttaaatattgaatcatagtactgtcatcttta
agttcttctgtctttcattgagtagtcaaataattggagtctggaattggtgtgaagatgatttatcaacaggtgctgctactccccgaa
catcacaggttcgtcattcttattactcaaggaatgttgtaaagaaattgacttttcttcttcagtgtgctttatctagtaactttttttttcca
g (SEQ ID NO: 16)
HSV-TK 3′
ggacgcggcggtggtaatgacaagcgcccagataacaatgggcatgccttatgccgtgaccgacgccgttctggctcctcatat
cgggggggaggctgggagctcacatgccccgcccccggccctcaccctcatcttcgaccgccatcccatcgccgccctcctgt
gctacccggccgcgcgataccttatgggcagcatgaccccccaggccgtgctggcgttcgtggccctcatcccgccgaccttg
cccggcacaaacatcgtgttgggggcccttccggaggacagacacatcgaccgcctggccaaacgccagcgccccggcgag
cggcttgacctggctatgctggccgcgattcgccgcgtttacgggctgcttgccaatacggtgcggtatctgcagggcggcggg
tcgtggcgggaggattggggacagctttcggggacggccgtgccgccccagggtgccgagccccagagcaacgcgggccc
acgaccccatatcggggacacgttatttaccctgtttcgggcccccgagttgctggcccccaacggcgacctgtacaacgtgttt
gcctgggccttggacgtcttggccaaacgcctccgtcccatgcacgtctttatcctggattacgaccaatcgcccgccggctgcc
gggacgccctgctgcaacttacctccgggatggtccagacccacgtcaccacccccggctccataccgacgatctgcgacctg
gcgcgcacgtttgcccgggagatgggggaggctaactga (SEQ ID NO: 6)
17) >Lenti_PGK_PURO_P2A_HSV-TK_MELK_GTF3C1_356 nt_ALT5′SS MUT<
HSV-TK 5′ with alt5′ss mutation
atggcttcgtacccctgccatcaacacgcgtctgcgttcgaccaggctgcgcgttctcgcggccatagcaaccgacgtacggcg
ttgcgccctcgccggcagcaagaagccacggaagtccgcctggagcagaaaatgcccacgctactgcgggtttatatagacg
gtcctcacgggatggggaaaaccaccaccacgcaactgctggtggccctgggttcgcgcgacgatatcgtctacgtacccgag
ccgatgacttactggcaggtgctgggggcttccgagacaatcgcgaacatctacaccacacaacaccgcctcgaccaggggg
agatatcggccgg (SEQ ID NO: 19)
MELK/GTF3C1 Combo 356 nt
gtgagttcagttccccaggccaagagcagctgagcggccaggcgcagcctccagagggctctgaagaccccagaggtaccg
cgcgccttgtcccccaccccacctcaccccaccctggctttcccgcgtgactgtattttaaatattgaatcatagtactgtcatcttta
agttcttctgtctttcattgagtagtcaaataattggagtctggaattggtgtgaagatgatttatcaacaggtgctgctactccccgaa
catcacaggttcgtcattcttattactcaaggaatgttgtaaagaaattgacttttcttcttcagtgtgctttatctagtaactttttttttcca
g (SEQ ID NO: 16)
HSV-TK 3′
ggacgcggcggtggtaatgacaagcgcccagataacaatgggcatgccttatgccgtgaccgacgccgttctggctcctcatat
cgggggggaggctgggagctcacatgccccgcccccggccctcaccctcatcttcgaccgccatcccatcgccgccctcctgt
gctacccggccgcgcgataccttatgggcagcatgaccccccaggccgtgctggcgttcgtggccctcatcccgccgaccttg
cccggcacaaacatcgtgttgggggcccttccggaggacagacacatcgaccgcctggccaaacgccagcgccccggcgag
cggcttgacctggctatgctggccgcgattcgccgcgtttacgggctgcttgccaatacggtgcggtatctgcagggcggcggg
tcgtggcgggaggattggggacagctttcggggacggccgtgccgccccagggtgccgagccccagagcaacgcgggccc
acgaccccatatcggggacacgttatttaccctgtttcgggcccccgagttgctggcccccaacggcgacctgtacaacgtgttt
gcctgggccttggacgtcttggccaaacgcctccgtcccatgcacgtctttatcctggattacgaccaatcgcccgccggctgcc
gggacgccctgctgcaacttacctccgggatggtccagacccacgtcaccacccccggctccataccgacgatctgcgacctg
gcgcgcacgtttgcccgggagatgggggaggctaactga (SEQ ID NO: 6)
18) >MSCV_EF1a_mCherry_P2A_mEmerald_MELK_GTF3C1_Combo_356 nt
mEmerald 5′
atggtgagcaagggcgaggagctgttcaccggggtggtgcccatcctggtcgagctggacggcgacgtaaacggccacaagt
tcagcgtgtccggcgagggcgagggcgatgccacctacggcaagctgaccctgaagttcatctgcaccaccggcaagctgcc
cgtgccctggcccaccctcgtgaccaccttgacctacggcgtgcagtgcttcgcccgctaccccgaccacatgaagcagcacg
acttcttcaagtccgccatgcccgaaggctacgtccaggagcgcaccatcttcttcaaggacgacggcaactacaagacccgcg
ccgaggtgaagttcgag (SEQ ID NO: 7)
MELK/GTF3C1 COMBO 356 NT
gtgagttcagttccccaggccaagagcagctgagcggccaggcgcagcctccagagggctctgaagaccccagaggtaccg
cgcgccttgtcccccaccccacctcaccccaccctggctttcccgcgtgactgtattttaaatattgaatcatagtactgtcatcttta
agttcttctgtctttcattgagtagtcaaataattggagtctggaattggtgtgaagatgatttatcaacaggtgctgctactccccgaa
catcacaggttcgtcattcttattactcaaggaatgttgtaaagaaattgacttttcttcttcagtgtgctttatctagtaactttttttttcca
g (SEQ ID NO: 16)
mEmerald 3′
ggcgacaccctggtgaaccgcatcgagctgaagggcatcgacttcaaggaggacggcaacatcctggggcacaagctggag
tacaactacaacagccacaaggtctatatcaccgccgacaagcagaagaacggcatcaaggtgaacttcaagacccgccacaa
catcgaggacggcagcgtgcagctcgccgaccactaccagcagaacacccccatcggcgacggccccgtgctgctgcccga
caaccactacctgagcacccagtccaagctgagcaaagaccccaacgagaagcgcgatcacatggtcctgctggagttcgtga
ccgccgccgggatcactctcggcatggacgagctgtacaagtaa (SEQ ID NO: 8)
19) >pcDNA3.1_CMV_ZNF19_378 nt_HSV-TK<
ZNF19 378 nt with ATG start site inserted within the synthetic intron
gcagccatgcctctgaaagctcaataccaggtgagctgtggatccctgaggtcagctctatggaggtctgagttgaaggacctag
gaaggttaactaataagagactttttttcctagggtttaaaaaatgttaaatcacattttatgacagtcaattttatatcctaatggaatct
cttctggtagtgagagaagttccagatttttcgttgtaaaaatagtcatataatggtctaatatccagttctgtcacattttctcattacct
atccctcacaggactttccctcaagtccctacctgtcccctctgccccacactctggcccggagccgccatgggattggctctgg
ggcagcaggaacctgttgttttag (SEQ ID NO: 17)
HSV-TK altered to remove ATG start site
tggcttcgtacccctgccatcaacacgcgtctgcgttcgaccaggctgcgcgttctcgcggccatagcaaccgacgtacggcgtt
gcgccctcgccggcagcaagaagccacggaagtccgcctggagcagaaaatgcccacgctactgcgggtttatatagacggt
cctcacgggatggggaaaaccaccaccacgcaactgctggtggccctgggttcgcgcgacgatatcgtctacgtacccgagcc
gatgacttactggcaggtgctgggggcttccgagacaatcgcgaacatctacaccacacaacaccgcctcgaccagggtgaga
tatcggccggggacgcggcggtggtaatgacaagcgcccagataacaatgggcatgccttatgccgtgaccgacgccgttctg
gctcctcatatcgggggggaggctgggagctcacatgccccgcccccggccctcaccctcatcttcgaccgccatcccatcgc
cgccctcctgtgctacccggccgcgcgataccttatgggcagcatgaccccccaggccgtgctggcgttcgtggccctcatccc
gccgaccttgcccggcacaaacatcgtgttgggggcccttccggaggacagacacatcgaccgcctggccaaacgccagcg
ccccggcgagcggcttgacctggctatgctggccgcgattcgccgcgtttacgggctgcttgccaatacggtgcggtatctgca
gggcggcgggtcgtggcgggaggattggggacagctttcggggacggccgtgccgccccagggtgccgagccccagagc
aacgcgggcccacgaccccatatcggggacacgttatttaccctgtttcgggcccccgagttgctggcccccaacggcgacct
gtacaacgtgtttgcctgggccttggacgtcttggccaaacgcctccgtcccatgcacgtctttatcctggattacgaccaatcgcc
cgccggctgccgggacgccctgctgcaacttacctccgggatggtccagacccacgtcaccacccccggctccataccgacg
atctgcgacctggcgcgcacgtttgcccgggagatgggggaggctaactga (SEQ ID NO: 20)
20) >pcDNA3.1_CMV_INTS3_411 nt_HSV-TK
INTS3 411 nt with ATG start site inserted within the synthetic intron
tgttaccgggacttagctctggtgagtcgtgatggcatgaatattgtcctgaataaaatcaaccagatacttatggagaagtacctg
aagctgcaggatacctgccgtactcaggtaaggccagaaagaaaagacaagatccagctcaaagagagaggatggatcttctc
tctgtcaggaacgggaaagaggaatcagggctaacacacccctatcattgtgtgtctaaattgtaatgtgctcctttcagttgtaatt
gaattagctcccttctcaaactcacagttcgccgccatgctcttcatctgtttttccctctttcctttagttggtgtggttggtacgggaa
ctggtgaagagtggggttctgggagccgatggtgtttgtatgacgtttatgaagcagattgc (SEQ ID NO: 18)
HSV-TK altered to remove ATG start site
gcttcgtacccctgccatcaacacgcgtctgcgttcgaccaggctgcgcgttctcgcggccatagcaaccgacgtacggcgttg
cgccctcgccggcagcaagaagccacggaagtccgcctggagcagaaaatgcccacgctactgcgggtttatatagacggtc
ctcacgggatggggaaaaccaccaccacgcaactgctggtggccctgggttcgcgcgacgatatgtctacgtacccgagcc
gatgacttactggcaggtgctgggggcttccgagacaatcgcgaacatctacaccacacaacaccgcctcgaccagggtgaga
tatcggccggggacgcggcggtggtaatgacaagcgcccagataacaatgggcatgccttatgccgtgaccgacgccgttctg
gctcctcatatcgggggggaggctgggagctcacatgccccgcccccggccctcaccctcatcttcgaccgccatcccatcgc
cgccctcctgtgctacccggccgcgcgataccttatgggcagcatgaccccccaggccgtgctggcgttcgtggccctcatccc
gccgaccttgcccggcacaaacatcgtgttgggggcccttccggaggacagacacatcgaccgcctggccaaacgccagcg
ccccggcgagcggcttgacctggctatgctggccgcgattcgccgcgtttacgggctgcttgccaatacggtgcggtatctgca
gggcggcgggtcgtggcgggaggattggggacagctttcggggacggccgtgccgccccagggtgccgagccccagagc
aacgcgggcccacgaccccatatcggggacacgttatttaccctgtttcgggcccccgagttgctggcccccaacggcgacct
gtacaacgtgtttgcctgggccttggacgtcttggccaaacgcctccgtcccatgcacgtctttatcctggattacgaccaatcgcc
cgccggctgccgggacgccctgctgcaacttacctccgggatggtccagacccacgtcaccacccccggctccataccgacg
atctgcgacctggcgcgcacgtttgcccgggagatgggggaggctaactga (SEQ ID NO: 21)

### Cell Culture

Isogenic K562, Marimo, and OCI-AML2 cells with and without defined SRSF2 mutations were cultured by methods well known in the art. In particular, K562 cells were grown at 37° C. and 5% atmospheric CO₂ in Iscove's Modified Dulbecco's Medium (IMDM; Gibco) supplemented with 10% fetal bovine serum (Gibco). Marimo cells were grown in RPMI and 10% FBS which OCI-AML2 cells were grown in 80 to 90% alpha-MEM (with ribo- and deoxyribonucleosides)+10-20% h.i. FBS.

### Example 3

This Example discloses additional embodiments of the synthetic intron platform and the applicability to screening assays to detect cells with aberrant RNA splicing and distinguish between cells without or with aberrant RNA splicing.

Specifically, reporter constructs were generated that implement fluorescent signals conditional upon aberrant RNA splicing (e.g., with mutation in the gene encoding SRAF2). FIGS. 7A and 7B are schematics of a fluorescent reporter construct with mEmerald interrupted by the synMELK 623 nt synthetic intron (SEQ D NO: 15). FIG. 7C is a schematic of a lentiviral vector with puromycin as the 1st coding sequence and selection marker and HSV-TK as the second coding sequence and the gene of interest.

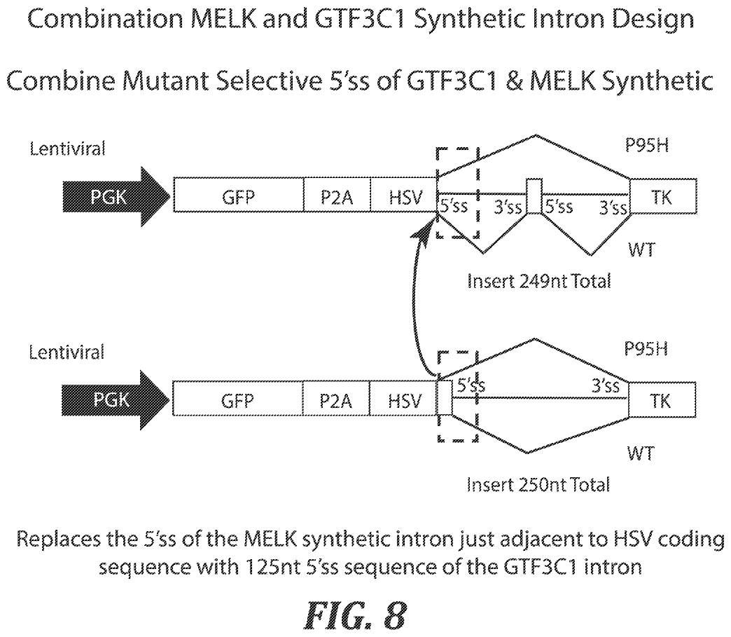

FIG. 8 is a schematic of a synthetic intron that comprises a combination of splicing elements, termed MELK and GTF3C1 splicing elements. The 250 nt GTF3C1 synthetic intron (SEQ ID NO: 11) was inserted and replaces the 5′ splice sites of the MELK synthetic intron just adjacent to the HSV coding sequence. The combined mutant synthetic intron was inserted into the coding sequence for HSV-TK.

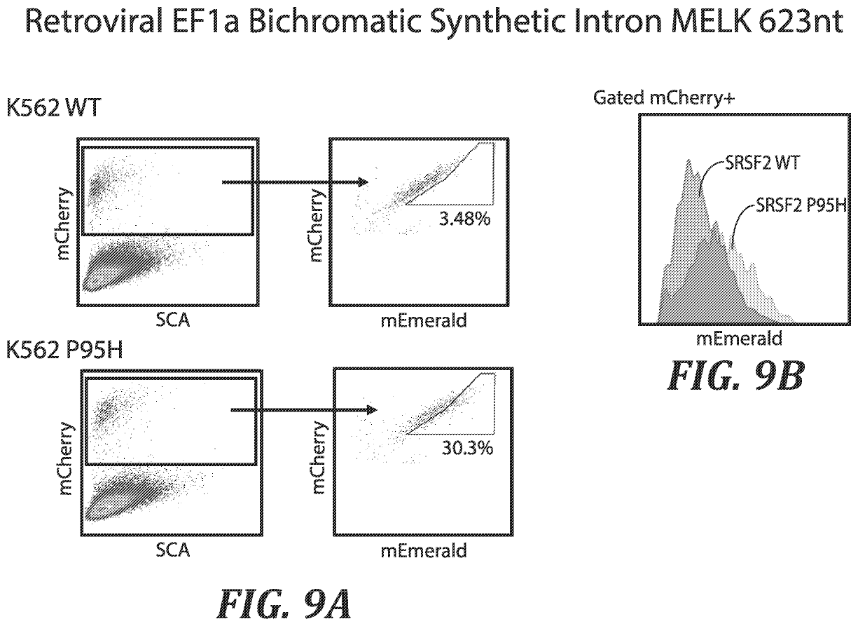

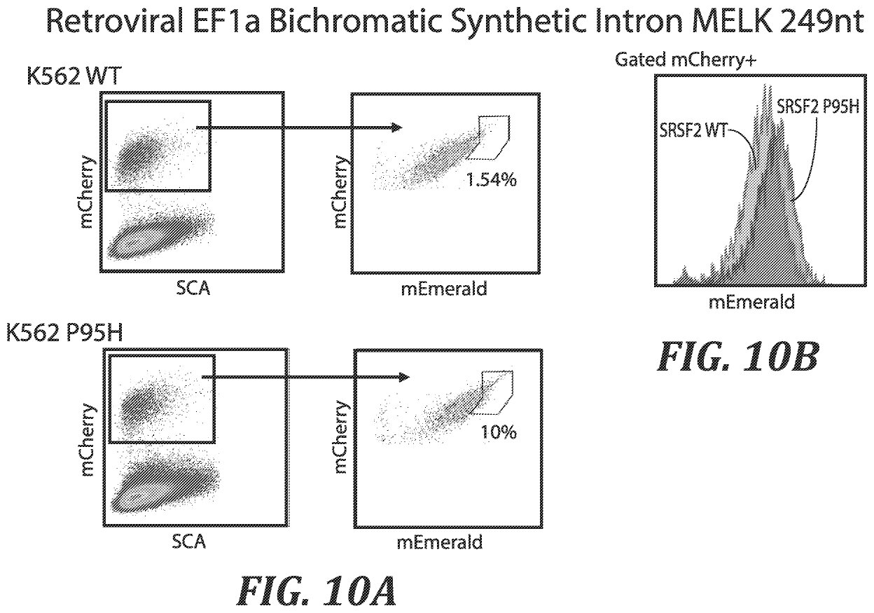

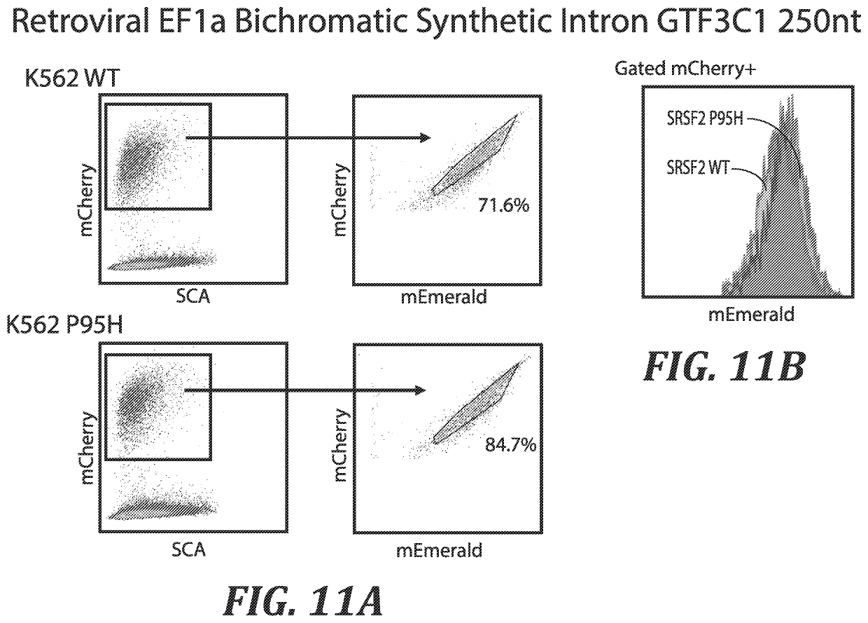

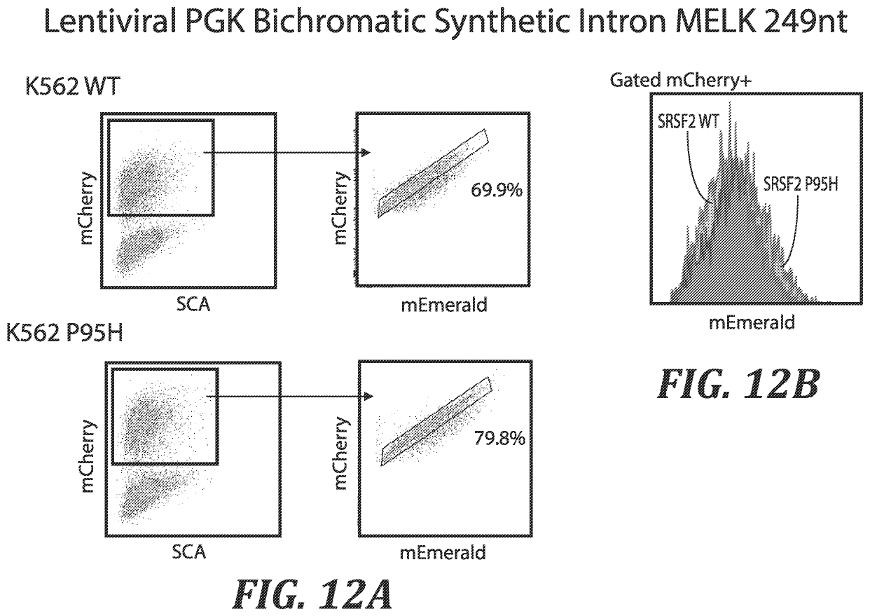

The mEmerald constructs of the Ef1a bichromatic synthetic intron MELK 623 nt, the retroviral Ef1 bichromatic synthetic intron MELK 249 nt, the retroviral Ef1 bichromatic synthetic intron GTF3C1_250 nt, and the lentiviral PGK bichromatic syntheticintron MELK 249 nt were introduced to K562 cells with either wild-type SRSF2 or mutated SRSF2 (P95H substitution), K562 cells with wild-type SRSF2, or K562 cells with mutated SRSF2 (P95H substitution). FIGS. 9A and 9B show flow cytometry plots of K562 cells transduced with the retroviral Ef1a bichromatic synthetic intron MELK 623 nt. FIGS. 10A and 10B show flow cytometry plots of K562 cells transduced with the retroviral Ef1a bichromatic synthetic intron MELK 249 nt. FIGS. 11A and 11B show flow cytometry plots of the retroviral Ef1a bichromatic synthetic intron GTF3C1. FIGS. 12A and 12B show flow cytometry plots of the lentiviral PGK bichromatic synthetic intron MELK 249 nt. In each of these figures mCherry+K562 cells (left plot) that are dichotomized by mCherry and GFP expression (right plot) in K562 cells transduced with the various retroviral synthetic intron constructs.

These results indicate a significant increase in conditional Emerald signal only in cells that contain SRSF2 mutations. Accordingly, the disclosed constructs can be used to distinguish cells with aberrant RNA splicing due to mutant SRSF2 activity. Such constructs are suitable for high-throughput screening of cells, for example, to screen for compositions and agents that antagonize mutations in the RNA splicing machinery (e.g., mutations in SRSF2).

### Example 4

In this example killing of cells that express the SRSF2 mutation and not wild type cells is demonstrated. Synthetic introns enabled mutation-dependent expression of herpes simplex virus thymidine kinase and subsequent ganciclovir-mediated elimination of SRSF2-mutant cancer cells, while leaving wild-type cells unaffected.

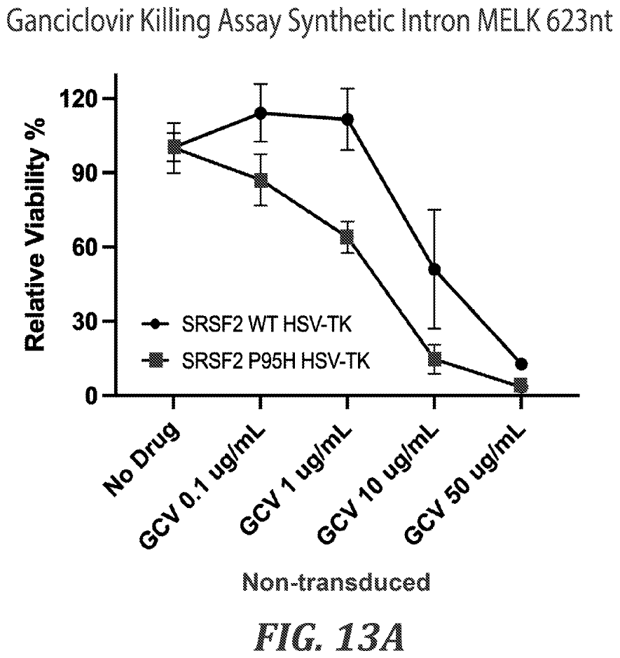

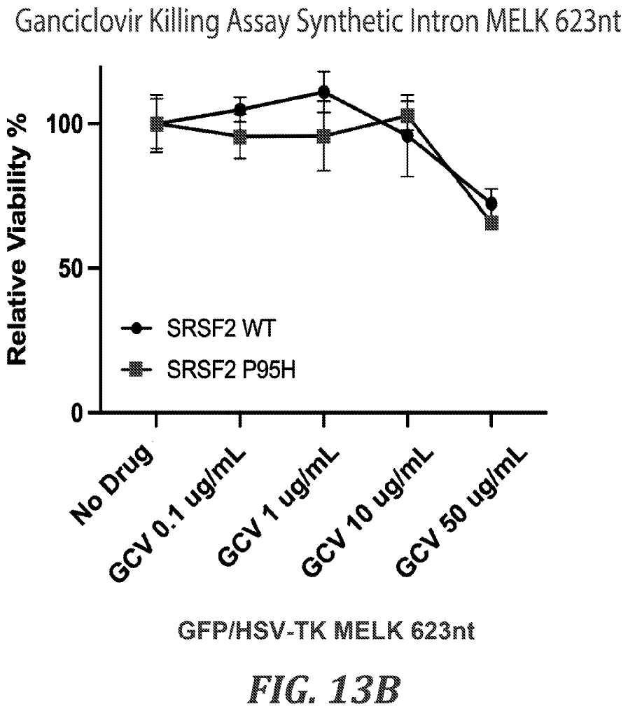

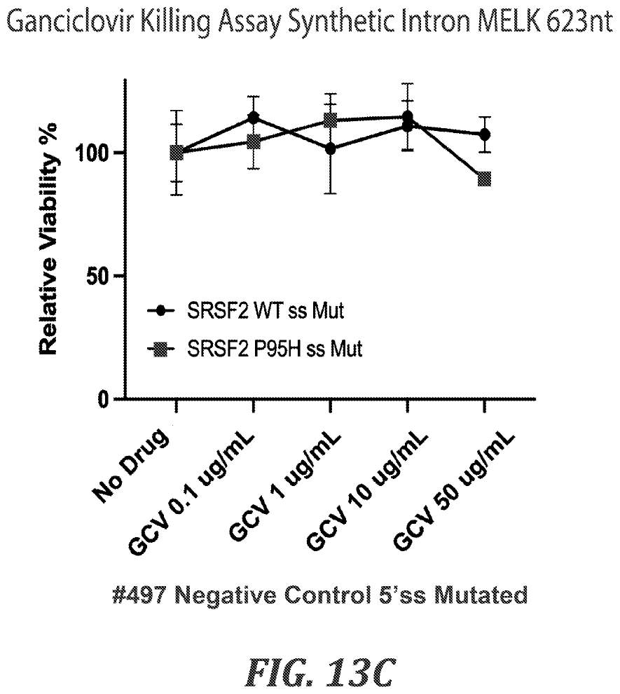

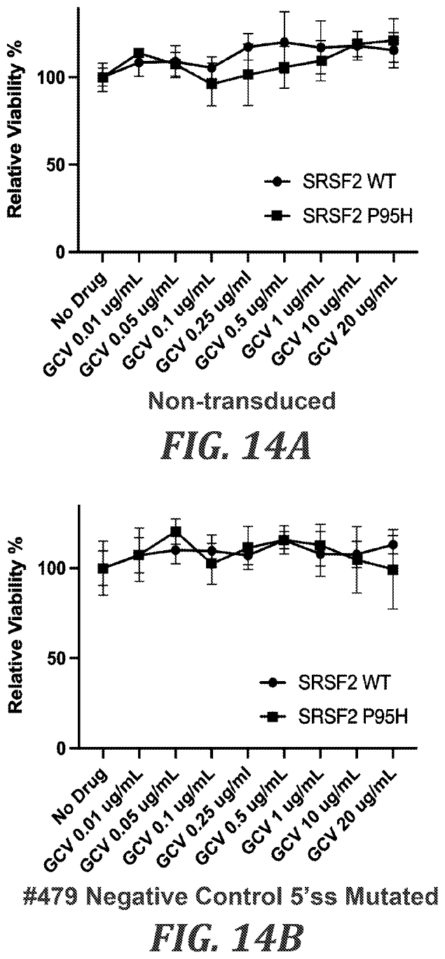

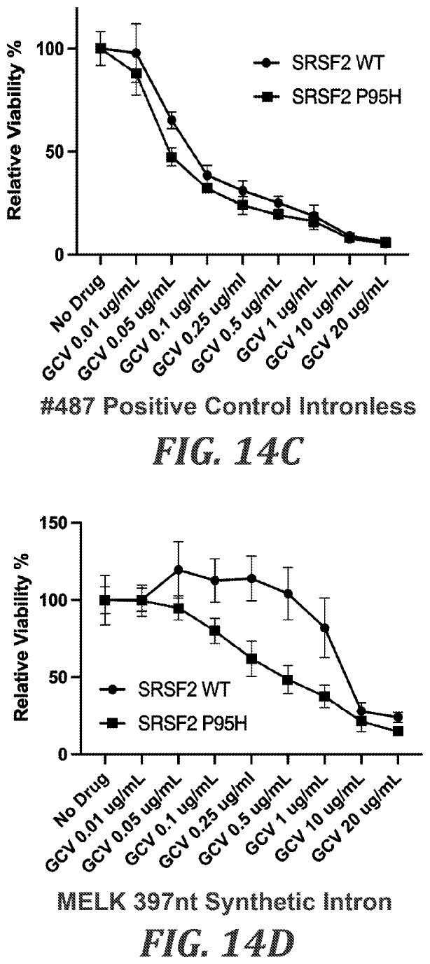

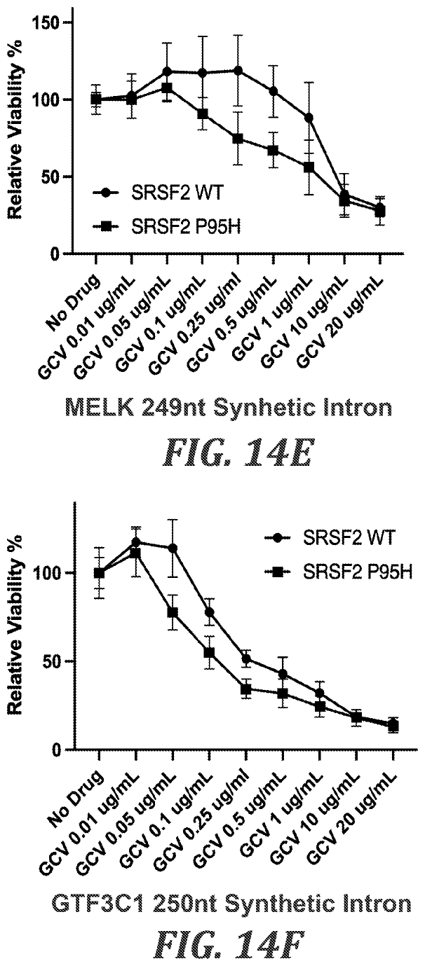

The relative viability of nontransduced and cells transduced with viral vectors comprising synthetic introns were tested for cell killing in vitro when ganciclovir are various concentrations is added. FIG. 13A-13C demonstrates the relative viability of non-transduced, GFP/HSV-TK MELK 623nt synthetic intron, or #497 negative control K562 isogenic cell lines treated with increasing concentrations of ganciclovir (GCV) in vitro. Cell viability was analyzed at day 11 of plating utilizing Cell Titer Glo Luminescent Cell Viability Assay. In another study, the relative viability of non-transduced, #497 negative control, or #487 positive control K562 isogenic cell lines treated with increasing concentrations of ganciclovir (GCV) in vitro. FIGS. 14A-14C. In FIGS. 14D-14F the relative viability of GFP/HSV-TK MELK 397nt synthetic intron, GFP/HSV-TK MELK 249nt synthetic intron, or GFP/HSV-TK GTF3C1 250nt synthetic intron K562 isogenic cell lines treated with increasing concentrations of ganciclovir (GCV) in vitro were exampled. Cell viability was analyzed at day 7 of plating utilizing Cell Titer Glo Luminescent Cell Viability Assay.

These studies demonstrate that the synthetic introns described herein demonstrate mutation-dependent expression of herpes simplex virus thymidine kinase and subsequent ganciclovir-mediated elimination of SRSF2-mutant cancer cells, while leaving wild-type cells unaffected.

TABLE 4
Endogenous Coding Sequences for the Human Genes for the Production of
Synthetic Introns.

1. Endogenous human MELK
MELK Intron 10 (SEQ ID NO: 22)
gtaagtgttactgcctgttgtgtcttgccacgtgccctccctgttcctgagtgtgtataccactgggaaggtaaactttccggtgtcaa
ggagtcagaggcaagatgttttatgatttcaatatttttgatgagaaaaatgggaagttctgaagttgaactctaattttttttttttttttttt
tttgagatagagtctcactctgtcatctaggctggagtgcagtggcatgacctcagctcactgcaacctctgcctccgggattcaag
cgattcttgtgcctcagcctcccgagtagctggaattgcaggtgtgcatcaccacgcctggccaatttttgtatctttagtagagacg
gggttttgccatgttggccaggctggtctcaaactcctgacctcaagtgatcagcctgcctcggcctcccaaagtgctgggattat
aggcatgagccaccgtgcccggcctctaaaattctgaatgttaacatggcattttacaaacctcctcccctgactccttttttttcataa
tagcaattcctgatggctatagaaaatgtttaagccaggccgggtgtggtggctcacacctgtaatcccagcactttgggaggcca
aggcaggtggatcacaaggtcaggagtttgacaccagcctggccaatatggtgaaaccccgtctgtactaaaaatacaaaaatta
gccaggcgtggtgatgagtgcctgtagtcccagctacttgggaggatgaggcaggagaatcgcttgaacccgagaggcggag
gttacagtgagctgagattgcgccattgcactccagcctgggcgacagagcaagactctgtctcaaaaaaaaaaaaaaaaaaaa
gaaaatgcttaagccacagatgagctaaaaggaaaatagaatttacctataaaccaactacctgtggaggatgtagttaatatttatt
gtgtctttccagtcttttttttaaagcatacttttacacgcattcaagtacttttaaaggacacattattgaattgcagagaacaaaaaaat
tgttgggagtaagatctgtgatatggaatatataatactttatatgacatgaaacattttgataaatattttcatatttatgttaagggttta
ggtttttgtttcttttaaaattttcatactgaaatggagcatgttgattagtgtttgttataaccaaattatttttctctacttcactgtctttgtc
tcagttttgtcatggactttaaaaaatttttttgttttattttttgaaacaaagtcttgctttgttgtcctggctggagtacagtggtgtgatc
atggctcactgcagccttaaccttccaggctcaagcaatcctcctgcctcagcctccctagtagctgggattacaggtgtgtgcca
ccacacctggctaatttttaattttctatagaaatggagtccagctatgttgcccaggctggtcttgaacacctgggctcaagcagtc
ctccctcctttgcctcccaaagtgctgggattattggcatgagtgactgcacctggcctgtcatggacatttaaaatggtgaagaag
cttacatacatagaggcaccatgtcattcctaaatgcttgtgttagtccttgttttgtgtgattctttgtatttcccatggcttaaaattcag
ctatccaaatactttgccagatgggtatggaactttgtaatgtcatcttccctcagattaattcaggttgcaatgtaaataatcttagca
ctaggaaattgtacctcatcctcagagaaaggtagttggttggccctgtttatgaaatccaagataacacaattgactgtgtggatg
aaagtttggtttttttcccctttataggcctcaaaactgtaaaaaaaaaacaaaaaatgttaaaatgccacctcttttatgatcttttgttg
aattccccctccctcaaattcctttaatatctatctcatttagatctccttttggtactcatcagtcatgtgctacaggtatagtgggtctta
tgtcactgccagaactaacctgcccctctttttccctccctagttggtcagtgcactggctgttgtgtaaagaaatctctgaagggag
atatcctgcatggttttctagtctgttgttggagagttttaatggctggtaaatattctgtgcctaggcagctcttttgcacatgggctcc
ttgaatcagccaatccctcatcccacccacactgccaaaccctttaaactttcaggtgcaagtaacacatctgggtccaggaaaat
gcaaaaccctgaattttgcatctgctcaaggtaattggagaaagctattagatacgtatacctctatgcttagaaaaatcactttcact
aaattttaaaatttacgacacctcagttaaggggcctgagacattttcctgagacattggattgtcttttttttttttttttttttttttgagaca
gagtcttgctttgttgcccaggctggagtgcagtagtgtggtcttggctcgctgcaacctccgcctcctgggttcaagtgattctcct
gcctcattgtcccgagtagctgggattacaggtgtgtgccaccacacctatctaatttttgtatttttagtagagatgggatttcaccat
gttggtcaggctggtctcgaactccagacctcagatgatccacctgcctcagcctcccaaagtgctgggaacacaagcgtgaac
caccacacccggcctggattttcttatatctgaattggcctggtctcaattgatctgcttatatgagttgtaagtggaaaagacctgga
aatatatttactcttatcttccatctactccctaatgaacatttaactaataatttaattatttgcaaagtcatattattgaatcaattttatgg
cagatcaagagacattttagtaacaagaagtgaaatagaaatcaaattttgggttcgcttctaatctttatgcaaattattgagacacg
aagcaagtcacatagtctggttgtactgttcttccccttgatagaattatcccttttctctggggtgttgtttataagaaagtaagtaatc
atgatcgatcaatcaatcaataggtggccctttcagtgaaacagcagtacagactgatccaagaatcacaaataaccacgtcatca
tcatcagtgtcattagcattttcaggccccatgctgggctgctgggaaacatatctgtgtaaagtataatacttgttctctaagagtca
agacatatatatacatagataatactgcatcatagtagatgatgggggccagagaagtggccagaggtgtcctggggagctttag
aggacaggaatgatcaccatgggatccaaactggaccaacagtcaaggttagaaagatggaaaagaagtggcacaacattca
gggcaagataaatggaatcagcaaaagcaggggcacctaattttgcttgatgtattctgttgacagtggctggacttgtgttaaaaa
agaagagttcaagtagggggagagagagagagagagagagagagaacgcaagtgagctggaagggtggacaagagccag
atagtagagaacctgaaaatgcacatcagaattttagactttgttcaatagttaatgaggagccattgatttgtgagcaagagagag
agaaatgaacatgattttgaaaaattaatccagtggtgtatacgattgtttagtcaggggtgatatgtgtgctattagtccctactgga
gtacccatgacagccgtcattcgtacagcactgtacttctccccaccaagcctagacttagctttaggatttttctcagcctagctctc
catgcatccagttccaaatgacagtaattggtaatcctggagaactctgtcatccttggttgagacagtggaaagattagtggtaga
gacaatattgtctgtttgaatagtctaggtatgagaagatttgtccttgatctaggatagtggcagtgagactttgggaaaaaaagga
tgagatataagaaattcaagtaaaattgcttggctagattaacaatatatcgagggaaaaggaaggaaggagaaataaaaactcc
aagtacacccacagtcttgaattcagggtataattagtatacccttagtttatagaggaggaagaatgaaagaacagaagttattttg
gtggcgttgttagcgcaagaccccaaatgtcctatccctaggagatccttcataaggctaattttcttcctggtagaaattattcacta
ccctgtatattcttacaaagaagaaacctatatttcgaagttgtttcccctccatcttctactttgtcagatctttttatttgtaaatttgcag
tagagttgtctggtattatagtgattatgtgataatgaaaagtagtatgctatataacataagaatgttgcaagacatattatttgagagt
cggaggttttcctttctgataatttgaactgaaattttctgacttcctttttctgtactggatggtcagctccttcaatatagtgtctcctatt
catttttgtttccagggccttgccctatgcctggaatacagttttgtgctgcataaagacaacgttggactgcatttatgatggtggtcc
cataaaattataataccatgtttttactctaccttttctttttttttctttcttttttttttagacagagtctcgctgtattacccaggctggagtg
cagtggcacaatttctgcttgttgtaacctccacctggtggactcaagtgatcctcccacctaagcctcccaagtacctgggcctac
aggcacatgcctggctaatttctattttttgtagagatggggtttcaccgtgttgcccagactggctccaacgcctgagcttaagtga
tccacctgtgtcagccttccaaagtgctgggattacaggcatgagccaccatggggacggcctttttctgtttagatttgtttagata
cataaatacttattgttgtgttagagccacctactgtattcagtacagtaacgtgctacacaggtttgtagtgtaggagcaacaggct
ataacgtacagcctaggtgtggcctaggtgtgtagtaggctctgacgtctcagtttgtgtaagtacattttatgatgttcacacagtga
taaaattgcctaatacatttctcagaatgtgtctctgtcgttaagtgacgcgtgactgtattttaaatattgaatcatagtactgtcatcttt
aagttcttctgtctttcattgagtag
MELK Exon 10 (SEQ ID NO: 23)
tcaaataattggagtctggaagatgtgaccgcaagtgataaaaattatgtggcgggattaatagactatgattggtgtgaagatgat
ttatcaacaggtgctgctactccccgaacatcacag
MELK Intron 11 (SEQ ID NO: 24)
gttcgtcattcttattactatttaaggcagaatctattgtttcaattataaaaacaaccagactaatgtgaaaccaaggtgtgcttgcaa
atttaatgaatgcttaagccactgaaaatgtttatgagcgataactggaatacgaatgaaagggaagtcaattatatccaagttgaa
gaaagccaagattctattttagagctctttactgctcctttatttcctgtttattattattattttttgagactgagtcttgctctgttggccag
gctggagtgcagtggcacgatctcggctcactgcaacctctgcctcccaggttcaagtgattctcatgcctcctgagtagctggga
ttacaggtgcctgctgcctctcctggctaatttttgtatttttagtagagacagggtttcaccatgttggcagctggtctcgaactcctg
acctcaggtgatccacccacctcagccttccaaagtgctgggattacaggcgtgagccactgtgcccggccttcttttttgtttaaa
atgaggtataatgaggtatactttacatgtagttaaataggtaaagtcaccttttttagtgtgcaattctatgtgttttgacaaatgcatac
agttgtataaccaccaccaaaattaagatataaaacagttccgttactcctcaaaattccctcattccccttgtggctaacttcccctc
ccaactctcggtggcaattgatctgtttttctttccttttgataatctttttattgtgactttaagtttgttgagctttttagattaatgaagatt
aagtatagattaatgttttttatcaaatttgggaagttttcaatattttatttgccccactctctttattttatcttctggtactccccttccatgt
atattggcacacttgatggtgccctacagtacactgaggccctgttcatttttctttatctattttcattctgtttctcagactaatttcagtt
aacctgtttcaaagtttcagattctttcttctgcttactgaaagttctgtttaatccctctagtgaattttttgttatggttattgtactcttgaa
ctccagaatttgttttgttcttttaaataattttatctttatttatactctctatttattgtgacattgctcttacactttcccttggttcttcagatg
tttccttttgttctttgaacatatttaaaaactgattttcagtctttgcttggtaaggcctacgtctggacttcttcaaggacaatttccattg
actgtttttgtttttctcgtgtatatgggccatcctttttgtttgtttgtttgttttttgtttgagacagaggctccgctgtatcgcccaggctg
gagcgcattgttgggatctctgctcactgcaacctctgcctcccgggttcaagcaattctcctgcctcagcctcctgagtagctggg
attacaggcacccgccatcatgcctggctaattttttttttttctattttttttttttttttgagacagagtctcgctctgtcacccaggctgg
actgcagtggcgctatcttggctcactgcaagctccgcctcctgggttcatgccattctcctgcctcagcttcccgaatagctggga
ctacaggtgcccgccaccacgcctggctaattttttgtatttttagtagagatggggtttcaccatgttagctaggatggtctcgatct
cctgaccttgtgatccgcccgcctcggcctcccaaagtgctgggattacaggcttgagccactgcacctggccttttttttgtgttttt
gtaaagatggggtttcaccatattggccaggctggtgttaaactcctgacctcaggtgatctgcctgccttagcctcccaaagtgct
gggattacaggcatgagccaccgtgcccggccatacttttttatttctttgcatgtatatgtcttgtaattgttgaaaactagacatttta
aataatataatgtggcagctctggtaatcagatttatcccctcctcaggatttgatattattgctgtttgttgttgttggttagtgacttttct
gaactaattctgtaaagtcgttaagtcatttaggtcagtgttgggctgcatttatgatggtggtccatgagattataatatcatatttttac
tataccttttctctgctcaattagcatagtgatcagctaatgcttggcgaggtttccttagatgcctggaatcaattcgtctctcagtcttt
gtgccggctctgtgtacatctggggtatgcctgcaactttcagccaggcagctgacaactctgcattagatttcatttcctatttgtat
aaaacctcaaggtcagcttgaggtgagatcttagatccttctaaagtgttttttgttttttatttttatttttttgagatggagttttgctcttgt
tgcccaggctggagtgcaatggcacgatctcagttcactgcgattttcgcctcctgggttcaagcaattctcctgcctcagcctccc
atggagctgggattacaggcatgcgccaccacacctggctaattttgtatttttagtagagacgaggtttcaccatgttggtcaggc
tggtctcgaactgctgacctcaagtgatccacctgcctcagcctcccaaagtgctgggattacaggcgtgagccaccatgccca
gccccttctaaagtctttcctgagcaaatgcacagccctgggtatgagcacatcctatacatgtacattgggcttgtagataccctg
gtatattttggagcttttccaattcactgtggacatcttattctctaacttttttttcagcatttttgttagtttattgtggtcaccagctattact
tactgcttcggcagccacaatgtgcaacaattgcctgtaaatttttttttgtttttgagataggatctcattccgtcaaccaggctggag
tccagtggtgtgatcatggctcactgcagcctcgacctcctgggcttaagcaattttcttgcctcagcctcccgagtatcggggact
ataggcatgcaccaccatgcccagctaatttttacaaaattattttgtagagacagggtctcaatattttgcccaggctggtctcaaa
ctcctggactcaagagatccacccgcgttggcctcccaaagtgctgggattacaggcgtgagccactgcgcctggcacctctaa
atctttttaacagatgctgctggggaaaaggctgttcatactgggcaggctctgagtaaggtcaaataaagacagctttcaggctg
ggtgcagtggctcacgcctgtaatcccagcactttggggggctgaggcgggtggatcacctgaggtcgggagttcaagaccta
cctgaccaacatggagaaaccccgtctctactaaaaatacaaaattagctgcctgtggtggcacatgcctgtaatcccagctactc
ggaaggctgaggcaggagaattgcttgaacccgggaggtggagtttgcagtgagctgagttcacgccattacactccagcctg
ggcaacaagagcaaaactccgtctcaaataaaaaacaaacaaagaataaaaaaagacagctttcatagtagggtcttctaggga
actactggccaggctaaataatgacagttttctgagcatgaagctttgaaggagctccaacctgttctgccccctccagcggctcct
aggttgctggtcttcatcgtgattgtgggctgtgggtttctaagactatcatagagctggagagagaagaatggggataggtaggg
caaattcaaacacctcaaactcactgtttctacttagctgttttctatgaataaatgctgcgatctttggctaatacacagagttctgaa
acaaaagaggtatactttgtttatggatagggagatttgaaggtatagatttcagtttttcccaaaatgaattttaaatttaatgtaatctc
aacttttcagtggatgtaaaataagtcaaaatgattctaaaatgtatgtggaaatacaaaggggcaagaataactaaatcattcctaa
agaataagaagatgggaggaaactggtcaacctgataaaagaactattataaaattaaagaaagtaagatttccttaataaataaa
acaatgaaacaaataaaacaatgaaacctttcataccttatatttaggtatgtctctttaaaaataacatagctaggccgggcgcggt
ggctcacgcctgtaatcccagcactttgggaggccaaggtgggtggatcatgaggtcaggagattgagaccatcctggctaaca
caatgaaactccatctctactaaaaaatacaaaaaattagctgggcatggtgggggcacctctagtcccagttacttgggaggct
gaggcaggagaatggcatgaacccgggaggcggagcttgcagtgagccgagattgcaccactgcactccagcctaggtgac
agagcgagactccgtctcaaaaaaaaaaaaaaaaaaaacaaaacaaacaaagaaaaaaacatagctagatttggtatgcaaac
tgaaaatcttgtcttttgaattgagcgcttacccaacttatttttattatggatttagttattatatatctttctttttttatgtttcctatgtgtctt
tttctgtatttcccccttatttcttgattttttatgttaatttccccccacattttattggtttctatttgatttctattcttttaatatttactctaaatt
tttaacatttaatttaacgaaatataaagtgaatctgtatctttttttttttttttttaagatggaatcttgctctgttgcccaggcttgagtgc
aatggcgtgatctcaggctcactgcaacctcttcctcctgggttcaagtgattctcctgcctcagcctcccaagtagccgggtttac
aggcacccgccaccatgtctggctaatttttgtatttttagtagagacagggtttcaccatgttggccaggctggtctcgaactcctg
acctcaggtgatccacctgcctcggcctcccaaagtgctagaattacaggcgtgagcccctgcaccctgctgtgaatctgtatgtt
taatttccttctaaacaatatgaaggccttagaacacttaatctctgattacccctcctggattccatgctattgatgtgtccagtgtatt
agctcttgaatgtttttaactttttattttgaaatatttatggattcacaggatgttattgaagatattattccactttcttcttgcttctattgtt
gctattgaaaaaccaacctcagtctagtgcctttttagtctgtcttttctctctggctccttttaggaccttctctttgcttttggtgttctgc
agtttcactgttagggctactgcgttgcacacctccagggaacacaccatctgtgtggtgtctctgtggatagtgtcctctggagttg
tataatctggtaaatagagatgtggatttcttttatgtatccagttcatgattcatcatgctttctatgtatggggatttgtatttttaatcaat
ttttgaaaatttcttgaataatatactgctttttctttttctccttttccttgtttcttttttttttttttttgagacagagtttctctcttcttgccca
ggctggagtgcaatggcacgatctcagcttactgcaacctctgcttcccaggttcaagcgattttcctacctcagcctcccgagta
gctgggattacaggtgcctgtccccatgcccagctaatttttgtattattagtagagacagagtttcaccatgttggccaggctggtc
ttgtactcctgacctcaggtgatcacatgacttggcctcccaaagtgctgggattacaggtgtgagccagcgcacccagcctgctt
ttctttaaaacaatatttatataaatttatagagtacaagtacaattttcttagatgcatagtggtcaagtgagaggttttaaggtgtcag
ctgaataatgtacatcgcaccccttaagtaatttctcatcaccaacccccttccatctcctcacccttccaagtctacattttctatcatt
ccactctctacatccacgtgtacacccttttttagcacccacttatgagtgagaacatgcaatatttgtcattctgtgtctggcttgtttc
acttaaggtaacctccacttccatccaccttgttgcaaaagataggatttcattcttttttatggttgaatagtattctatctgtgtgtctgt
ctgtctatctatctcacatttcctttatgcagtcatctgttgatggacacttgggttgattttgtatctttgctattgtgaataagtactgag
ataaatatacaagtgcaggtatgtttttgatgtattgatttcttttcctttgggaaaagaagtagagggaattgctgcttttgcattgtcca
tactatgtatatctggaacttagaatagccttatgttagacattttatttcattttctgtttccttcaaccatctttcacgtatcgtatgtcttttt
ctgtcttggctacattcagagttatttcggatttatatttctattaaaatatctctatttcttttattctgttatttgacatgtgcattgcacttttc
atttttatcatgtttttatttctagaagtttttattctttttcaaatctatttgaacagttttggtagtcttttgttctttactcatgtttttgtttcact
attttaaacatgcaaacatacttatttatgttctgtatttgataattttaatatctgaaatctttatgctcatagtgcttgtgtcttttggaatttt
ttatgtaggtttatttttctttgaactctatgggaattatctgaggcttgggttaagggcttgagttgggttggagtgaagcttttctttca
ggtaggagttgtgcgtgcttctaccaggttcctggggcacaacttagcataggactactttaagttaaatttaggcttgaggtttctg
ggaccatgcaggttgtattaatttggacctttatttatctgaagactggcctgtgtttacttacaaattattaaaagaaacttcatttttctc
ctccctcagaaccaagaccaagacaccctttttcttgcctattccctttgaagagcagatttatatttaactcagcctttaattttagcta
tagcccttcctggctttacatgggagctttggttctaactcaccaccttgcagaggcccaaggcttctgtggctgtagaaacacatg
ctataggttatcagaggcagaagatagctgcccttagggcagcagagagtttcagccttggttactcctttggattcattctcatgttt
ttagtttggcctctcaggattttacttactctcttgcaagctcaaccataaatttaaaaggattaaaaagtattttttatccaatatttctgtt
agagtagtttaacatgttttttctgccctaattgctgactaaactttttttttggtacatgtcagaggcaagccgtgactatctaggtatg
gaaagcatttcttagtgattgatttgtggccattcacaatattgcctaggatctgagtatgtgtatacactcagcctgtatcatgttgaa
aattactgtcaatagaacagactaaagtgatctacatgtattaaagaaaactatatattttcaaatttcctaatgtcaataatatccttttg
ataattcattttaataaaaatagtacaaggtagtttgcaaatgatcaaggaatgttgtaaagaaattgacttttcttcttcagtgtgcttta
tctagtaactttttttttccag
2) Endogenous human GTF3C1
GFT3C1 Intron 34 (SEQ ID NO: 25)
gtgagttcagttccccaggccaagagcagctgagcggccaggcgcagcctccagagggctctgaagaccccagaggtaccg
cgcgccttgtcccccaccccacctcaccccaccctggctttccctcccctccgccctgatgggcctcacaccttcctccgaggat
gggggacctgccactggcacaaaaggggcttgacttcagcttctcagagcctgagccctgagggggaggatactgcctgtaaa
tgcatttccaggggagggcaggcctcccacatccccaggcagcactgtgagttccagagcaggagctcaggcggtggggcca
gcgtgtgcactgcagcggctgctgtggggcagtgatggcatgggggtctcaacttcctcgggtggcagcacgccctttcctttcc
tgcacgtctgtcatcagcagcggccttctcatgtggcttctctgggcgtttttgttttctttggggtgtttttgcctcgggttttataccac
ccttgtggtcggggcttctgggtggaaaatgaggtgttcgctggcttcctgcaatgtggggcttgtccgggcctgggacgctgctt
gcatgtggcgctgtgtgcggcagtctgcccggccctgccacctccagggtggccgctctgtgagatgctcattgtcctttttttttg
acacttcatcccctgagcctcgaggtgccctgctcatgctaggtaccatgctgaaggttggagggcagagatggcttccctgactt
tccgagtctctcacattctcttcctcttccctcag
3. Endogenous human ARFIP2
ARFIP2 Intron 1 (SEQ ID NO: 26)
gtgagaggaacatgcttgggcgacgggaagttgaacgcacaaacctgtccagagggcaagatgccccgagccccggggaa
ggatgaggacacacctgatgtccaggtgtatgggggtgggggcggggactcacacacctgggagacataactgactgtggaa
gggtcaccgatatcctgggagagagaggcttttaccagagactgggaacatacacccactgatctaactaaggcctggtgggg
agggcccgaggaagacgaggtgtatgagacggaggaggggagaccccctgaaggaagggggaagaaacgctaaaggaat
gtgaaaggccaagcaatggagaacaaacctgaataggggggtgaagacctgagccctggaaagggttcatctcctcagcata
aacacgcctgcatggagataagttctaagagagcagactttccctcttcactgttgtattttcattgtcacaaatagtgcctgacacat
agtaggcactcactaaatgtctgctgaatgagtgtagaaataatcaccagagtcctggtgaggttgggggcttgtgagaagacac
cgtgctttggtgtgcctgtttcaaatgtgcgttttggaggggagaaatacatatatctgaagaaaggaatgatggccaaacgctgg
gacactcaaaccctgggacagcctttaaagaaatgacagaggaggcctccttcttctagagtgctggccagtgtccagaacttct
gtgttgggctttgcagggtgctggggtggagagttttactccaatacctttcctag
4. Endogenous human INTS3
INTS3 Intron 4 (SEQ ID NO: 27)
gtaaggccagaaagaaaagacaagatccagctcaaagagagaggatggatcttctctctgtcaggaacgggaaagaggaatc
agggctaacacacccctatcattgtgtgtctaaattgtaatgtgctcctttcagttgtaattgaattagctcccttctcaaactcacagtt
cctgctcttcatctgtttttccctctttcctttag
INTS3 Exon 5 (SEQ ID NO: 28)
ttggtgtggttggtacgggaactggtgaagagtggggttctgggagccgatggtgtttgtatgacgtttatgaagcagattgcag
5. Endogenous ZNF19
ZNF19 Exon 3 (SEQ ID NO: 29)
ggtacccagttcccaaacctgcactgatctcacttttggagagaggggacatggcttggggcctggaagcacaggatgatcccc
cagcagagaggaccaaaaacgtctgtaaag
ZNF19 Intron 3 (SEQ ID NO: 30)
gtaggtgaggcagatttagaaccaacaattgtagcgttttatggagcactgtattttaaaatgaatcatgctgggcaggatttaaata
aaatagatgatttcacctaataggtaaaatatgaaacgtttctcaccaatcttaactcttaccaacatgtaatcactcagtacgtctcat
ctgcaatccttgaatccattgttcctttctgagtttcaacattgcctaccttagttgtcctccttggttgggatggatttattcaggagca
gtttttgagcaacttgccatgtgctgaacgttgtttaagaagttctggcaggtacaagttatggagacagatgtaaaaagcgaaaaa
gaaaagtattaagtgctgttgggagtgggacggagaatgtgaaggtaaaatgggccagatcttcccaaaagttggccataaatg
gggagagctgagtcagataacactaggaagtcatggcatgggaggagctttttgaattcataggaattcagagctgagcacagg
gcgtgaggtgcctttagaataggcaagtggatggctagagcaggccattgagaaggtgggtggagagtctgagataaaaggca
ggtctgaaacagtactctaacacagctgggttgaaacgatccccaggaagaaaaaatgaaggctgaatcttaaccattaagggat
atttaatttcagtgagagaaggaagagaaagtagacagtcaaatagatgggggccaccaagagcaaacagtgaaattgaaggc
agaggcaggagtttcagtgcaggtggtttgcagagagcaaatacgtggaaaataatgaagctagcacagcacttcagcctcttat
tctaagaatctgaaaaagaatcatcaagacaattgagaacctgttggtttgttctcacacgcttcctctgtctggtgattattctttttca
gactcttggttaccttgtttacttcaatctcagattactctcctctgaacattctccttcggagttcccttactcctttaactcacactcata
ggatgaacttgattaaccatcacatgagtcccaggtaacccacattaatgttaaaaccaagcccatcatttcatataaaatggattga
ctttaactcatttaaaatgtcatccaatcttttggaacacaaatgccattttatgggctgtttgtctcctttttgctgataggagaaagaat
actctcagtctgggtgtgtctaagtaaactgttcatcccttttcaaatagagagtctaggacagtagtttctctgcatgtttgaggttca
gcagttttagggcgtttcctcatcgtctatagtccatgtgtggttctgcagttaagaacctctgaactaggtaatcttacaacatattgg
gaataatactctctaagcacttctgtgattttctttctctctgtcttgccacaacccagtgtctgtaatgttcagtgtcttaagtcttttggg
acatctgcagggtatgagtttttaaaaagaacaacaattcatcctttgcctttgctttctgatactgttctgtttcactttctgattctaagt
cttgccagccccgatgtcactacttgttcttaagacctgtgccttctttggcattttgtaggctgtaagtagcatcaattcaaaagatac
tagctcatgccagggatgaccagacatcattcaaatattagagtagaaaagcttaggaagcagtctcgtgtgttgtgtccactgata
ccctgttccacctcaatgttctctgcctgtgtgatatttggcactttcactttaacagccttttcttcccagcatgctgatgtgaaagttg
aaatgatctcttttaaagccttatcattttcaatgctctattatttctagtatagttctcaggtggcattatttgtgttttctatctcttatgtca
g
ZNF19 Exon 4 (SEQ ID NO: 31)
atgttgagaccaacattgacagtgagtccacattaatccagggaatttctgaagaaagagatgggatgatgtcacatggtcagctg
aagagtgtccctcagagaactgacttcccagaaacacgtaatgtggaaaagcaccaggacatccccacagtgaaaaatatccaa
ggaaaggttccaagaatcccctgtgcaaggaaacctttcatatgtgaggagtgtgggaaatccttcagctacttttcttactatgcta
gacaccagagaatccacactggggagaaaccttttgagtgtagtgagtgtggaaaagcctttaatggtaattcttcgttaattcggc
accagaggattcacactggagagagaccctatcagtgtgaggagtgtgggcgagcctttaatgataatgcaaatctgatcaggc
atcagagaatccacagtggggacagaccctattactgtacagagtgtgggaatagtttcacgagtagttccgagtttgttatacatc
agagaatccacactggggagaaaccctatgagtgtaatgagtgtggcaaagcttttgttggtaattcacccctacttcggcatcag
aaaatccacactggagagaaaccctatgagtgtaatgagtgtggcaaaagctttggaaggacttcccatctaagccaacatcagc
gtattcacacaggggaaaagccttattcttgtaaagtatgtggacaagccttcaattttcatacaaaactaactcggcaccagagaa
ttcacagtgaggagaaaccctttgactgtgtagattgtggaaaagccttcagtgctcaggaacaattaaaaaggcatctgagaatt
catactcaggagtcttcctatgtatgtgatgagtgtggaaaagccttgactagcaaaagaaatcttcatcagcatcaaagaatccat
actggagagaaaccctatgagtgtagcaagtatgagaaggcctttgggacttcttcccagctaggtcaccttgagcatgtctactct
ggagagaagcctgtgctggacatttgtcgttttggcctcccagaattttttacccccttttactggtaa

While illustrative embodiments have been illustrated and described, it will be appreciated that various changes can be made therein without departing from the spirit and scope of the invention.

1. An artificial nucleic acid intron construct, comprising an intron comprising:
(i) an upstream flanking exon;
(ii) an upstream intron;
(iii) an alternatively spliced cassette exon;
(iv) a downstream intron; and
(v) a downstream flanking exon.

2. The artificial nucleic acid intron construct of claim 1, wherein the construct comprises a Kozak sequence at the 5′ end and an alternatively spliced cassette exon flanked by upstream and downstream introns; an intron comprising at least one cryptic 5′ splice site; an intron comprising at least one cryptic 3′ splice site; or an intron that is alternatively retained, wherein the intron is derived from a human wild type intro selected from a human wild type intron selected from intron 10, exon 10, and intron 11, intron 12, exon 12, and/or intron 13 of human MELK, intron 34 of human GTF3C1, intron 1 of human ARFIP2, exon 4, intron 4, and/or exon 5 of human INTS3, or exon 3 and intron 3 of human ZNF19; or combinations thereof.

3. The artificial nucleic acid intron construct of claim 1, wherein the intron is at least about 50 nucleotides to about 1000 nucleotides in length.

4. (canceled)

5. The artificial nucleic acid intron construct of claim 1, wherein the human wild type intron from which the intron is derived is one of the following:
intron 10 of MELK comprising a sequence set forth in SEQ ID NO:22;
exon 10 of MELK comprising a sequence set forth in SEQ ID NO:10;
intron 11 of MELK comprising a sequence set forth in SEQ ID NO:24;
intron 12 of MELK comprising a sequence set forth in SEQ ID NO:1;
exon 12 of MELK comprising a sequence set forth in SEQ ID NO:2;
intron 13 of MELK comprising a sequence set forth in SEQ ID NO:3
intron 34 of GTF3C1 comprising a sequence set forth in SEQ ID NO:25;
intron 1 of ARFIP2 comprising a sequence set forth in SEQ ID NO:26;
intron 4 of INTS3 comprising a sequence set forth in SEQ ID NO:27;
exon 5 of INTS3 comprising a sequence set forth in SEQ ID NO:28;
exon 3 of ZNF19 comprising a sequence set for in SEQ ID NO:29;
intron 3 of ZNF19 comprising a sequence set forth in SEQ ID NO:30;
exon 4 of ZNF19 comprising a sequence set for in SEQ ID NO:31;
or combinations thereof.

6. The artificial nucleic acid intron construct of claim 1 wherein the construct comprises:
(i) the first 50 nt of MELK intron 10, the last 200 nt of MELK intron 10, 123 nt of endogenous MELK exon 10, the first 50 nt of MELK intron 11, and the last 200 nt of MELK intron 11;
(ii) the first 125 nt of GTF3C1 intron 34, and the last 200 nt of GTF3C1 intron 34;
(iii) the first 50 nt of ARFIP2 intron 1 and the last 200 nt of ARFIP2 intron 1;
(iv) all 114 nt of the INTS3 exon 4, and 174 nt of INTS3 intron 4; or
(v) all 30 nt of ZNF19 exon 3, the first 50 nt of ZNF19 intron 3, the last 200 nt of ZNF19 intron 3, the first 55 nt of the ZNF19 alternative splice sequence, the nucleotides GCCATG, and 3 nucleotides to code for a methionine residue, the remaining 37 nt of the ZNF19 alternative splice sequence, and a modified coding sequence of HSV-TK.

7. The artificial nucleic acid intron construct of claim 1, wherein the intron or exon has a 5′ end domain with about 10 to about 150 nucleotides having at least 50% sequence identity to a sequence of the 5′-most 10 to about 150 nucleotides of the wild type intron.

8. The artificial nucleic acid intron construct of claim 1, wherein the intron or exon has a 3′ end domain with about 50 to about 350 nucleotides having at least 50% sequence identity to a sequence of the 3′-most 50 to about 350 nucleotides of the wild type intron.

9. The artificial nucleic acid intron construct of claim 1,
wherein the intron or exon has a sequence with at least 75% sequence identity to a sequence selected from SEQ ID NOS: 1-3 and 22-31.

10. The artificial nucleic acid intron construct of claim 1, wherein the canonical 5′ splice site comprises a sequence selected from GTGAG, GTAAG, GTGCG, GTACG, GTGGG, GTAGG, GTGTG, GTATG, and GTATC, or wherein the canonical 3′ splice site comprises a sequence selected from AAG, CAG, TAG, ATG, CTG, GTG, and TTG.

11. (canceled)

12. The artificial nucleic acid intron construct of claim 1, wherein the at least one cryptic 3′ splice site comprises a GT dinucleotide immediately followed by a consensus 5′ splice site context optional, wherein the consensus 5′ splice site context is selected from GTGAG, GTAAG, GTGCG, GTACG, GTGGG, GTAGG, GTGTG, GTATG, and GTATC.

13. The artificial nucleic acid intron construct of claim 1, wherein the intron comprises a plurality of cryptic 3′ splice sites within about 100 nucleotides upstream of the canonical 3′ splice site or within about 100 nucleotides downstream of the canonical 3′ splice site, and wherein each of the plurality of the canonical 3′ splice sites comprises an AG dinucleotide immediately preceded by a C or a T and wherein the canonical 3′ splice sequence is independently selected from AAG, CAG, GAG, and TAG.

14. The artificial nucleic acid intron construct of claim 1, wherein the intron comprises an insertion, deletion, or mutation of SSNC nucleic acid sequences, wherein S=C or G.

15. The artificial nucleic acid intron construct of claim 1, wherein the coding sequence is modified to encode an exonic splicing enhancer or an exonic splicing silencer, wherein the exonic splicing enhancer comprises CCNG, GGNG, CGNG, GCNG and the exonic splicing silencer comprises TTTGTTCCGT (SEQ ID NO:32) or GGGTGGTTTA (SEQ ID NO:33), GTAGGTAGGT (SEQ ID NO: 34), TTCGTTCTGC (SEQ ID NO:35), GGTAAGTAGG (SEQ ID NO:36), GGTTAGTTTA (SEQ ID NO:37), TTCGTAGGTA (SEQ ID NO: 38), GGTCCACTAG (SEQ ID NO:39), TTCTGTTCCT (SEQ ID NO:40), TCGTTCCTTA (SEQ ID NO:41), GGGATGGGGT (SEQ ID NO:42), GTTTGGGGGT (SEQ ID NO:43), TATAGGGGGG (SEQ ID NO:44), GGGGTTGGGA (SEQ ID NO:45), TTTCCTGATG (SEQ ID NO: 46), TGTTTAGTTA (SEQ ID NO:47), TTCTTAGTTA (SEQ ID NO:48), GTAGGTTTG, GTTAGGTATA (SEQ ID NO:49), TAATAGTTTA (SEQ ID NO:50), or TTCGTTTGGG (SEQ ID NO: 51).

16. (canceled)

17. The artificial nucleic acid intron construct of claim 1, wherein the intron is configured to be spliced differently in a cancer cell comprising a change-of-function or loss-of-function mutation in SRSF2 relative to the splicing pattern of the intron in a cell lacking a change-of-function or loss-of-function mutation in SRSF2.

18. (canceled)

19. The artificial nucleic acid intron construct of claim 1, further comprising a first exon domain and a second exon domain, wherein the intron is disposed between the first exon domain and the second exon domain, the combination of the first exon domain and the second exon domain without the intron encodes part or all of a protein of interest.

20-36. (canceled)

37. A method of selectively expressing, or alternately selectively not expressing, a gene of interest in a cell, wherein the cell comprises a change-of-function or loss-of-function mutation in a recurrently mutated RNA splicing factor gene, the method comprising:
introducing to the cell an expression cassette comprising a coding sequence (CDS) interrupted by at least one artificial nucleic acid intron as recited in claim 1, wherein the expression cassette further comprises a promoter operatively linked to the CDS; and
permitting transcription of the coding sequence and modified splicing of the transcript induced by the artificial nucleic acid intron in the resulting transcript in conjunction with the mutated splicing factor, the cell is a cancer cell and the mutation in the RNA splicing factor gene is a change-of-function or loss-of-function mutation in SFSR2.

38-39. (canceled)

40. The method of claim 37, wherein the cancer is a myelodysplastic syndrome (MDS), chronic myelomonocytic leukemia (CMML), acute myeloid leukemia (AML), myeloproliferative neoplasms (MDN), uveal melanoma, bladder cancer, lung adenocarcinoma, or other neoplasms with a recurrent SRSF2 mutation.

41-42. (canceled)

43. A method of treating a subject with cancer, wherein the cancer is characterized by a change-of-function or loss-of-function mutation in a recurrently mutated RNA splicing factor gene, the method comprising:
administering to the subject an effective amount of a therapeutic composition comprising an expression cassette comprising a gene transcript comprising a coding sequence (CDS) interrupted by at least one artificial nucleic acid intron as recited in claim 1, wherein the expression cassette further comprises a promoter operatively linked to the CDS, and wherein upon splicing of the at least one artificial nucleic acid intron from the gene transcript in a cancer cell the CDS encodes a functional therapeutic protein.

44. (canceled)

45. The method of claim 43, wherein the cancer is selected from a myelodysplastic syndrome (MDS), chronic myelomonocytic leukemia (CMML), acute myeloid leukemia (AML), myeloproliferative neoplasms (MDN), uveal melanoma, bladder cancer, lung adenocarcinoma, and other neoplasms with a recurrent SRSF2 mutation.

46. (canceled)

47. The method of claim 43, wherein the functional therapeutic protein is a toxin, a chemokine, a cytokine, a growth factor, a targetable cell-surface protein, a targetable antigen, a druggable enzyme, and a detectable marker.

48-80. (canceled)


SEQUENCE LISTING (51 sequences)

SEQ ID NO: 1 - DNA, 250 bp, Homo sapiens
  source 1..250: mol_type = other DNA
gtaagtgttactgcctgttgtgtcttgccacgtgccctccctgttcctgacagcctaggtgtggcctaggtgtgtagtaggctctgacgtctcagtttgtgtaagtacattttatgatgttcacacagtgataaaattgcctaatacatttctcagaatgtgtctctgtcgttaagtgacgcgtgactgtattttaaatattgaatcatagtactgtcatctttaagttcttctgtctttcattgagtag

SEQ ID NO: 2 - DNA, 123 bp, Homo sapiens
  source 1..123: mol_type = other DNA
tcaaataattggagtctggaagatgtgaccgcaagtgataaaaattatgtggcgggattaatagactatgattggtgtgaagatgatttatcaacaggtgctgctactccccgaacatcacag

SEQ ID NO: 3 - DNA, 250 bp, Homo sapiens
  source 1..250: mol_type = other DNA
gttcgtcattcttattactatttaaggcagaatctattgtttcaattatatgtcaatagaacagactaaagtgatctacatgtattaaagaaaactatatattttcaaatttcctaatgtcaataatatccttttgataattcattttaataaaaatagtacaaggtagtttgcaaatgatcaaggaatgttgtaaagaaattgacttttcttcttcagtgtgctttatctagtaactttttttttccag

SEQ ID NO: 4 - DNA, 455 bp, Homo sapiens
  source 1..455: mol_type = other DNA
gatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacagccaagttttacatgcccaagaaggccacagaactgaaacatcttcagtgtctagaagaagaactcaaacctctggaggaagtgctaaatttagctcaaagcaaaaactttcacttcgaccccagggacgtggtgagcaatatcaacgtattcgttctggaactaaagggatctgaaacaacattcatgtgtgaatatgctgatgagacagcaaccattgtagaatttctgaacagatggattacctttgtctcaaagcatcatctcaacactgacttga

SEQ ID NO: 5 - DNA, 345 bp, Herpes simplex virus
  source 1..345: mol_type = other DNA
atggcttcgtacccctgccatcaacacgcgtctgcgttcgaccaggctgcgcgttctcgcggccatagcaaccgacgtacggcgttgcgccctcgccggcagcaaggaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccgg

SEQ ID NO: 6 - DNA, 787 bp, Herpes simplex virus
  source 1..787: mol_type = other DNA
ggacgcggcggtggtaatgacaagcgcccagataacaatgggcatgccttatgccgtgaccgacgccgttctggctcctcatatcgggggggaggctgggagctcacatgccccgcccccggccctcaccctcatcttcgacctccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactga

SEQ ID NO: 7 - DNA, 348 bp, synthetic construct
  source 1..348: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgag

SEQ ID NO: 8 - DNA, 372 bp, synthetic construct
  source 1..372: mol_type = other DNA
ggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 9 - DNA, 150 bp, synthetic construct
  source 1..150: mol_type = other DNA
gtgagaggaacatgcttgggcgacgggaagttgaacgcacaaacctgtccgaggcctccttcttctagagtgctggccagtgtccagaacttctgtgttgggctttgcagggtgctggggtggagagttttactccaatacctttcctag

SEQ ID NO: 10 - DNA, 250 bp, synthetic construct
  source 1..250: mol_type = other DNA
gtgagaggaacatgcttgggcgacgggaagttgaacgcacaaacctgtcctgcgttttggaggggagaaatacatatatctgaagaaaggaatgatggccaaacgctgggacactcaaaccctgggacagcctttaaagaaatgacagaggaggcctccttcttctagagtgctggccagtgtccagaacttctgtgttgggctttgcagggtgctggggtggagagttttactccaatacctttcctag

SEQ ID NO: 11 - DNA, 250 bp, synthetic construct
  source 1..250: mol_type = other DNA
gtgagttcagttccccaggccaagagcagctgagcggccaggcgcagcctccagagggctctgaagaccccagaggtaccgcgcgccttgtcccccaccccacctcaccccaccctggctttccctttgacacttcatcccctgagcctcgaggtgccctgctcatgctaggtaccatgctgaaggttggagggcagagatggcttccctgactttccgagtctctcacattctcttcctcttccctcag

SEQ ID NO: 12 - DNA, 325 bp, synthetic construct
  source 1..325: mol_type = other DNA
gtgagttcagttccccaggccaagagcagctgagcggccaggcgcagcctccagagggctctgaagaccccagaggtaccgcgcgccttgtcccccaccccacctcaccccaccctggctttccccgctgtgtgcggcagtctgcccggccctgccacctccagggtggccgctctgtgagatgctcattgtcctttttttttgacacttcatcccctgagcctcgaggtgccctgctcatgctaggtaccatgctgaaggttggagggcagagatggcttccctgactttccgagtctctcacattctcttcctcttccctcag

SEQ ID NO: 13 - DNA, 249 bp, synthetic construct
  source 1..249: mol_type = other DNA
gtaagtgttactgcctgtgcgtgactgtattttaaatattgaatcatagtactgtcatctttaagttcttctgtctttcattgagtagtcaaataattggagtctggaattggtgtgaagatgatttatcaacaggtgctgctactccccgaacatcacaggttcgtcattcttattactcaaggaatgttgtaaagaaattgacttttcttcttcagtgtgctttatctagtaactttttttttccag

SEQ ID NO: 14 - DNA, 397 bp, synthetic construct
  source 1..397: mol_type = other DNA
gtaagtgttactgcctgttgtgtcttaagtacattttatgatgttcacacagtgataaaattgcctaatacatttctcagaatgtgtctctgtcgttaagtgacgcgtgactgtattttaaatattgaatcatagtactgtcatctttaagttcttctgtctttcattgagtagtcaaataattggagtctggaattggtgtgaagatgatttatcaacaggtgctgctactccccgaacatcacaggttcgtcattcttattactatttaaatatccttttgataattcattttaataaaaatagtacaaggtagtttgcaaatgatcaaggaatgttgtaaagaaattgacttttcttcttcagtgtgctttatctagtaactttttttttccag

SEQ ID NO: 15 - DNA, 623 bp, synthetic construct
  source 1..623: mol_type = other DNA
gtaagtgttactgcctgttgtgtcttgccacgtgccctccctgttcctgacagcctaggtgtggcctaggtgtgtagtaggctctgacgtctcagtttgtgtaagtacattttatgatgttcacacagtgataaaattgcctaatacatttctcagaatgtgtctctgtcgttaagtgacgcgtgactgtattttaaatattgaatcatagtactgtcatctttaagttcttctgtctttcattgagtagtcaaataattggagtctggaagatgtgaccgcaagtgataaaaattatgtggcgggattaatagactatgattggtgtgaagatgatttatcaacaggtgctgctactccccgaacatcacaggttcgtcattcttattactatttaaggcagaatctattgtttcaattatatgtcaatagaacagactaaagtgatctacatgtattaaagaaaactatatattttcaaatttcctaatgtcaataatatccttttgataattcattttaataaaaatagtacaaggtagtttgcaaatgatcaaggaatgttgtaaagaaattgacttttcttcttcagtgtgctttatctagtaactttttttttccag

SEQ ID NO: 16 - DNA, 356 bp, synthetic construct
  source 1..356: mol_type = other DNA
gtgagttcagttccccaggccaagagcagctgagcggccaggcgcagcctccagagggctctgaagaccccagaggtaccgcgcgccttgtcccccaccccacctcaccccaccctggctttcccgcgtgactgtattttaaatattgaatcatagtactgtcatctttaagttcttctgtctttcattgagtagtcaaataattggagtctggaattggtgtgaagatgatttatcaacaggtgctgctactccccgaacatcacaggttcgtcattcttattactcaaggaatgttgtaaagaaattgacttttcttcttcagtgtgctttatctagtaactttttttttccag

SEQ ID NO: 17 - DNA, 378 bp, synthetic construct
  source 1..378: mol_type = other DNA
gcagccatgcctctgaaagctcaataccaggtgagctgtggatccctgaggtcagctctatggaggtctgagttgaaggacctaggaaggttaactaataagagactttttttcctagggtttaaaaaatgttaaatcacattttatgacagtcaattttatatcctaatggaatctcttctggtagtgagagaagttccagatttttcgttgtaaaaatagtcatataatggtctaatatccagttctgtcacattttctcattacctatccctcacaggactttccctcaagtccctacctgtcccctctgccccacactctggcccggagccgccatgggattggctctggggcagcaggaacctgttgttttag

SEQ ID NO: 18 - DNA, 411 bp, synthetic construct
  source 1..411: mol_type = other DNA
tgttaccgggacttagctctggtgagtcgtgatggcatgaatattgtcctgaataaaatcaaccagatacttatggagaagtacctgaagctgcaggatacctgccgtactcaggtaaggccagaaagaaaagacaagatccagctcaaagagagaggatggatcttctctctgtcaggaacgggaaagaggaatcagggctaacacacccctatcattgtgtgtctaaattgtaatgtgctcctttcagttgtaattgaattagctcccttctcaaactcacagttcgccgccatgctcttcatctgtttttccctctttcctttagttggtgtggttggtacgggaactggtgaagagtggggttctgggagccgatggtgtttgtatgacgtttatgaagcagattgc

SEQ ID NO: 19 - DNA, 344 bp, synthetic construct
  source 1..344: mol_type = other DNA
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggggagatatcggccgg

SEQ ID NO: 20 - DNA, 1130 bp, synthetic construct
  source 1..1130: mol_type = other DNA
tggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactga

SEQ ID NO: 21 - DNA, 1128 bp, synthetic construct
  source 1..1128: mol_type = other DNA
gcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaactga

SEQ ID NO: 22 - DNA, 5363 bp, Homo sapiens
  source 1..5363: mol_type = other DNA
gtaagtgttactgcctgttgtgtcttgccacgtgccctccctgttcctgagtgtgtataccactgggaaggtaaactttccggtgtcaaggagtcagaggcaagatgttttatgatttcaatatttttgatgagaaaaatgggaagttctgaagttgaactctaattttttttttttttttttttttgagatagagtctcactctgtcatctaggctggagtgcagtggcatgacctcagctcactgcaacctctgcctccgggattcaagcgattcttgtgcctcagcctcccgagtagctggaattgcaggtgtgcatcaccacgcctggccaatttttgtatctttagtagagacggggttttgccatgttggccaggctggtctcaaactcctgacctcaagtgatcagcctgcctcggcctcccaaagtgctgggattataggcatgagccaccgtgcccggcctctaaaattctgaatgttaacatggcattttacaaacctcctcccctgactccttttttttcataatagcaattcctgatggctatagaaaatgtttaagccaggccgggtgtggtggctcacacctgtaatcccagcactttgggaggccaaggcaggtggatcacaaggtcaggagtttgacaccagcctggccaatatggtgaaaccccgtctgtactaaaaatacaaaaattagccaggcgtggtgatgagtgcctgtagtcccagctacttgggaggatgaggcaggagaatcgcttgaacccgagaggcggaggttacagtgagctgagattgcgccattgcactccagcctgggcgacagagcaagactctgtctcaaaaaaaaaaaaaaaaaaaagaaaatgcttaagccacagatgagctaaaaggaaaatagaatttacctataaaccaactacctgtggaggatgtagttaatatttattgtgtctttccagtcttttttttaaagcatacttttacacgcattcaagtacttttaaaggacacattattgaattgcagagaacaaaaaaattgttgggagtaagatctgtgatatggaatatataatactttatatgacatgaaacattttgataaatattttcatatttatgttaagggtttaggtttttgtttcttttaaaattttcatactgaaatggagcatgttgattagtgtttgttataaccaaattatttttctctacttcactgtctttgtctcagttttgtcatggactttaaaaaatttttttgttttattttttgaaacaaagtcttgctttgttgtcctggctggagtacagtggtgtgatcatggctcactgcagccttaaccttccaggctcaagcaatcctcctgcctcagcctccctagtagctgggattacaggtgtgtgccaccacacctggctaatttttaattttctatagaaatggagtccagctatgttgcccaggctggtcttgaacacctgggctcaagcagtcctccctcctttgcctcccaaagtgctgggattattggcatgagtgactgcacctggcctgtcatggacatttaaaatggtgaagaagcttacatacatagaggcaccatgtcattcctaaatgcttgtgttagtccttgttttgtgtgattctttgtatttcccatggcttaaaattcagctatccaaatactttgccagatgggtatggaactttgtaatgtcatcttccctcagattaattcaggttgcaatgtaaataatcttagcactaggaaattgtacctcatcctcagagaaaggtagttggttggccctgtttatgaaatccaagataacacaattgactgtgtggatgaaagtttggtttttttcccctttataggcctcaaaactgtaaaaaaaaaacaaaaaatgttaaaatgccacctcttttatgatcttttgttgaattccccctccctcaaattcctttaatatctatctcatttagatctccttttggtactcatcagtcatgtgctacaggtatagtgggtcttatgtcactgccagaactaacctgcccctctttttccctccctagttggtcagtgcactggctgttgtgtaaagaaatctctgaagggagatatcctgcatggttttctagtctgttgttggagagttttaatggctggtaaatattctgtgcctaggcagctcttttgcacatgggctccttgaatcagccaatccctcatcccacccacactgccaaaccctttaaactttcaggtgcaagtaacacatctgggtccaggaaaatgcaaaaccctgaattttgcatctgctcaaggtaattggagaaagctattagatacgtatacctctatgcttagaaaaatcactttcactaaattttaaaatttacgacacctcagttaaggggcctgagacattttcctgagacattggattgtcttttttttttttttttttttttttgagacagagtcttgctttgttgcccaggctggagtgcagtagtgtggtcttggctcgctgcaacctccgcctcctgggttcaagtgattctcctgcctcattgtcccgagtagctgggattacaggtgtgtgccaccacacctatctaatttttgtatttttagtagagatgggatttcaccatgttggtcaggctggtctcgaactccagacctcagatgatccacctgcctcagcctcccaaagtgctgggaacacaagcgtgaaccaccacacccggcctggattttcttatatctgaattggcctggtctcaattgatctgcttatatgagttgtaagtggaaaagacctggaaatatatttactcttatcttccatctactccctaatgaacatttaactaataatttaattatttgcaaagtcatattattgaatcaattttatggcagatcaagagacattttagtaacaagaagtgaaatagaaatcaaattttgggttcgcttctaatctttatgcaaattattgagacacgaagcaagtcacatagtctggttgtactgttcttccccttgatagaattatcccttttctctggggtgttgtttataagaaagtaagtaatcatgatcgatcaatcaatcaataggtggccctttcagtgaaacagcagtacagactgatccaagaatcacaaataaccacgtcatcatcatcagtgtcattagcattttcaggccccatgctgggctgctgggaaacatatctgtgtaaagtataatacttgttctctaagagtcaagacatatatatacatagataatactgcatcatagtagatgatgggggccagagaagtggccagaggtgtcctggggagctttagaggacaggaatgatcaccatgggatccaaactggaccaacagtcaaggttagaaagatggaaaagaagtggcacaacattcagggcaagataaatggaatcagcaaaagcaggggcacctaattttgcttgatgtattctgttgacagtggctggacttgtgttaaaaaagaagagttcaagtagggggagagagagagagagagagagagagaacgcaagtgagctggaagggtggacaagagccagatagtagagaacctgaaaatgcacatcagaattttagactttgttcaatagttaatgaggagccattgatttgtgagcaagagagagagaaatgaacatgattttgaaaaattaatccagtggtgtatacgattgtttagtcaggggtgatatgtgtgctattagtccctactggagtacccatgacagccgtcattcgtacagcactgtacttctccccaccaagcctagacttagctttaggatttttctcagcctagctctccatgcatccagttccaaatgacagtaattggtaatcctggagaactctgtcatccttggttgagacagtggaaagattagtggtagagacaatattgtctgtttgaatagtctaggtatgagaagatttgtccttgatctaggatagtggcagtgagactttgggaaaaaaaggatgagatataagaaattcaagtaaaattgcttggctagattaacaatatatcgagggaaaaggaaggaaggagaaataaaaactccaagtacacccacagtcttgaattcagggtataattagtatacccttagtttatagaggaggaagaatgaaagaacagaagttattttggtggcgttgttagcgcaagaccccaaatgtcctatccctaggagatccttcataaggctaattttcttcctggtagaaattattcactaccctgtatattcttacaaagaagaaacctatatttcgaagttgtttcccctccatcttctactttgtcagatctttttatttgtaaatttgcagtagagttgtctggtattatagtgattatgtgataatgaaaagtagtatgctatataacataagaatgttgcaagacatattatttgagagtcggaggttttcctttctgataatttgaactgaaattttctgacttcctttttctgtactggatggtcagctccttcaatatagtgtctcctattcatttttgtttccagggccttgccctatgcctggaatacagttttgtgctgcataaagacaacgttggactgcatttatgatggtggtcccataaaattataataccatgtttttactctaccttttctttttttttctttcttttttttttagacagagtctcgctgtattacccaggctggagtgcagtggcacaatttctgcttgttgtaacctccacctggtggactcaagtgatcctcccacctaagcctcccaagtacctgggcctacaggcacatgcctggctaatttctattttttgtagagatggggtttcaccgtgttgcccagactggctccaacgcctgagcttaagtgatccacctgtgtcagccttccaaagtgctgggattacaggcatgagccaccatggggacggcctttttctgtttagatttgtttagatacataaatacttattgttgtgttagagccacctactgtattcagtacagtaacgtgctacacaggtttgtagtgtaggagcaacaggctataacgtacagcctaggtgtggcctaggtgtgtagtaggctctgacgtctcagtttgtgtaagtacattttatgatgttcacacagtgataaaattgcctaatacatttctcagaatgtgtctctgtcgttaagtgacgcgtgactgtattttaaatattgaatcatagtactgtcatctttaagttcttctgtctttcattgagtag

SEQ ID NO: 23 - DNA, 75 bp, Homo sapiens
  source 1..75: mol_type = other DNA
tcaaataattggagtctggaagatgtgaccgcaagtgataaaaattatgtggcgggattaatagactatgattgg

SEQ ID NO: 24 - DNA, 7986 bp, Homo sapiens
  source 1..7986: mol_type = other DNA
gttcgtcattcttattactatttaaggcagaatctattgtttcaattataaaaacaaccagactaatgtgaaaccaaggtgtgcttgcaaatttaatgaatgcttaagccactgaaaatgtttatgagcgataactggaatacgaatgaaagggaagtcaattatatccaagttgaagaaagccaagattctattttagagctctttactgctcctttatttcctgtttattattattattttttgagactgagtcttgctctgttggccaggctggagtgcagtggcacgatctcggctcactgcaacctctgcctcccaggttcaagtgattctcatgcctcctgagtagctgggattacaggtgcctgctgcctctcctggctaatttttgtatttttagtagagacagggtttcaccatgttggcagctggtctcgaactcctgacctcaggtgatccacccacctcagccttccaaagtgctgggattacaggcgtgagccactgtgcccggccttcttttttgtttaaaatgaggtataatgaggtatactttacatgtagttaaataggtaaagtcaccttttttagtgtgcaattctatgtgttttgacaaatgcatacagttgtataaccaccaccaaaattaagatataaaacagttccgttactcctcaaaattccctcattccccttgtggctaacttcccctcccaactctcggtggcaattgatctgtttttctttccttttgataatctttttattgtgactttaagtttgttgagctttttagattaatgaagattaagtatagattaatgttttttatcaaatttgggaagttttcaatattttatttgccccactctctttattttatcttctggtactccccttccatgtatattggcacacttgatggtgccctacagtacactgaggccctgttcatttttctttatctattttcattctgtttctcagactaatttcagttaacctgtttcaaagtttcagattctttcttctgcttactgaaagttctgtttaatccctctagtgaattttttgttatggttattgtactcttgaactccagaatttgttttgttcttttaaataattttatctttatttatactctctatttattgtgacattgctcttacactttcccttggttcttcagatgtttccttttgttctttgaacatatttaaaaactgattttcagtctttgcttggtaaggcctacgtctggacttcttcaaggacaatttccattgactgtttttgtttttctcgtgtatatgggccatcctttttgtttgtttgtttgttttttgtttgagacagaggctccgctgtatcgcccaggctggagcgcattgttgggatctctgctcactgcaacctctgcctcccgggttcaagcaattctcctgcctcagcctcctgagtagctgggattacaggcacccgccatcatgcctggctaattttttttttttctattttttttttttttttgagacagagtctcgctctgtcacccaggctggactgcagtggcgctatcttggctcactgcaagctccgcctcctgggttcatgccattctcctgcctcagcttcccgaatagctgggactacaggtgcccgccaccacgcctggctaattttttgtatttttagtagagatggggtttcaccatgttagctaggatggtctcgatctcctgaccttgtgatccgcccgcctcggcctcccaaagtgctgggattacaggcttgagccactgcacctggccttttttttgtgtttttgtaaagatggggtttcaccatattggccaggctggtgttaaactcctgacctcaggtgatctgcctgccttagcctcccaaagtgctgggattacaggcatgagccaccgtgcccggccatacttttttatttctttgcatgtatatgtcttgtaattgttgaaaactagacattttaaataatataatgtggcagctctggtaatcagatttatcccctcctcaggatttgatattattgctgtttgttgttgttggttagtgacttttctgaactaattctgtaaagtcgttaagtcatttaggtcagtgttgggctgcatttatgatggtggtccatgagattataatatcatatttttactataccttttctctgctcaattagcatagtgatcagctaatgcttggcgaggtttccttagatgcctggaatcaattcgtctctcagtctttgtgccggctctgtgtacatctggggtatgcctgcaactttcagccaggcagctgacaactctgcattagatttcatttcctatttgtataaaacctcaaggtcagcttgaggtgagatcttagatccttctaaagtgttttttgttttttatttttatttttttgagatggagttttgctcttgttgcccaggctggagtgcaatggcacgatctcagttcactgcgattttcgcctcctgggttcaagcaattctcctgcctcagcctcccatggagctgggattacaggcatgcgccaccacacctggctaattttgtatttttagtagagacgaggtttcaccatgttggtcaggctggtctcgaactgctgacctcaagtgatccacctgcctcagcctcccaaagtgctgggattacaggcgtgagccaccatgcccagccccttctaaagtctttcctgagcaaatgcacagccctgggtatgagcacatcctatacatgtacattgggcttgtagataccctggtatattttggagcttttccaattcactgtggacatcttattctctaacttttttttcagcatttttgttagtttattgtggtcaccagctattacttactgcttcggcagccacaatgtgcaacaattgcctgtaaatttttttttgtttttgagataggatctcattccgtcaaccaggctggagtccagtggtgtgatcatggctcactgcagcctcgacctcctgggcttaagcaattttcttgcctcagcctcccgagtatcggggactataggcatgcaccaccatgcccagctaatttttacaaaattattttgtagagacagggtctcaatattttgcccaggctggtctcaaactcctggactcaagagatccacccgcgttggcctcccaaagtgctgggattacaggcgtgagccactgcgcctggcacctctaaatctttttaacagatgctgctggggaaaaggctgttcatactgggcaggctctgagtaaggtcaaataaagacagctttcaggctgggtgcagtggctcacgcctgtaatcccagcactttggggggctgaggcgggtggatcacctgaggtcgggagttcaagacctacctgaccaacatggagaaaccccgtctctactaaaaatacaaaattagctgcctgtggtggcacatgcctgtaatcccagctactcggaaggctgaggcaggagaattgcttgaacccgggaggtggagtttgcagtgagctgagttcacgccattacactccagcctgggcaacaagagcaaaactccgtctcaaataaaaaacaaacaaagaataaaaaaagacagctttcatagtagggtcttctagggaactactggccaggctaaataatgacagttttctgagcatgaagctttgaaggagctccaacctgttctgccccctccagcggctcctaggttgctggtcttcatcgtgattgtgggctgtgggtttctaagactatcatagagctggagagagaagaatggggataggtagggcaaattcaaacacctcaaactcactgtttctacttagctgttttctatgaataaatgctgcgatctttggctaatacacagagttctgaaacaaaagaggtatactttgtttatggatagggagatttgaaggtatagatttcagtttttcccaaaatgaattttaaatttaatgtaatctcaacttttcagtggatgtaaaataagtcaaaatgattctaaaatgtatgtggaaatacaaaggggcaagaataactaaatcattcctaaagaataagaagatgggaggaaactggtcaacctgataaaagaactattataaaattaaagaaagtaagatttccttaataaataaaacaatgaaacaaataaaacaatgaaacctttcataccttatatttaggtatgtctctttaaaaataacatagctaggccgggcgcggtggctcacgcctgtaatcccagcactttgggaggccaaggtgggtggatcatgaggtcaggagattgagaccatcctggctaacacaatgaaactccatctctactaaaaaatacaaaaaattagctgggcatggtggcgggcacctctagtcccagttacttgggaggctgaggcaggagaatggcatgaacccgggaggcggagcttgcagtgagccgagattgcaccactgcactccagcctaggtgacagagcgagactccgtctcaaaaaaaaaaaaaaaaaaaacaaaacaaacaaagaaaaaaacatagctagatttggtatgcaaactgaaaatcttgtcttttgaattgagcgcttacccaacttatttttattatggatttagttattatatatctttctttttttatgtttcctatgtgtctttttctgtatttcccccttatttcttgattttttatgttaatttccccccacattttattggtttctatttgatttctattcttttaatatttactctaaatttttaacatttaatttaacgaaatataaagtgaatctgtatctttttttttttttttttaagatggaatcttgctctgttgcccaggcttgagtgcaatggcgtgatctcaggctcactgcaacctcttcctcctgggttcaagtgattctcctgcctcagcctcccaagtagccgggtttacaggcacccgccaccatgtctggctaatttttgtatttttagtagagacagggtttcaccatgttggccaggctggtctcgaactcctgacctcaggtgatccacctgcctcggcctcccaaagtgctagaattacaggcgtgagcccctgcaccctgctgtgaatctgtatgtttaatttccttctaaacaatatgaaggccttagaacacttaatctctgattacccctcctggattccatgctattgatgtgtccagtgtattagctcttgaatgtttttaactttttattttgaaatatttatggattcacaggatgttattgaagatattattccactttcttcttgcttctattgttgctattgaaaaaccaacctcagtctagtgcctttttagtctgtcttttctctctggctccttttaggaccttctctttgcttttggtgttctgcagtttcactgttagggctactgcgttgcacacctccagggaacacaccatctgtgtggtgtctctgtggatagtgtcctctggagttgtataatctggtaaatagagatgtggatttcttttatgtatccagttcatgattcatcatgctttctatgtatggggatttgtatttttaatcaatttttgaaaatttcttgaataatatactgctttttctttttctccttttccttgtttctttttttttttctttttgagacagagtttctctcttcttgcccaggctggagtgcaatggcacgatctcagcttactgcaacctctgcttcccaggttcaagcgattttcctacctcagcctcccgagtagctgggattacaggtgcctgtccccatgcccagctaatttttgtattattagtagagacagagtttcaccatgttggccaggctggtcttgtactcctgacctcaggtgatcacatgacttggcctcccaaagtgctgggattacaggtgtgagccagcgcacccagcctgcttttctttaaaacaatatttatataaatttatagagtacaagtacaattttcttagatgcatagtggtcaagtgagaggttttaaggtgtcagctgaataatgtacatcgcaccccttaagtaatttctcatcaccaacccccttccatctcctcacccttccaagtctacattttctatcattccactctctacatccacgtgtacacccttttttagcacccacttatgagtgagaacatgcaatatttgtcattctgtgtctggcttgtttcacttaaggtaacctccacttccatccaccttgttgcaaaagataggatttcattcttttttatggttgaatagtattctatctgtgtgtctgtctgtctatctatctcacatttcctttatgcagtcatctgttgatggacacttgggttgattttgtatctttgctattgtgaataagtactgagataaatatacaagtgcaggtatgtttttgatgtattgatttcttttcctttgggaaaagaagtagagggaattgctgcttttgcattgtccatactatgtatatctggaacttagaatagccttatgttagacattttatttcattttctgtttccttcaaccatctttcacgtatcgtatgtctttttctgtcttggctacattcagagttatttcggatttatatttctattaaaatatctctatttcttttattctgttatttgacatgtgcattgcacttttcatttttatcatgtttttatttctagaagtttttattctttttcaaatctatttgaacagttttggtagtcttttgttctttactcatgtttttgtttcactattttaaacatgcaaacatacttatttatgttctgtatttgataattttaatatctgaaatctttatgctcatagtgcttgtgtcttttggaattttttatgtaggtttatttttctttgaactctatgggaattatctgaggcttgggttaagggcttgagttgggttggagtgaagcttttctttcaggtaggagttgtgcgtgcttctaccaggttcctggggcacaacttagcataggactactttaagttaaatttaggcttgaggtttctgggaccatgcaggttgtattaatttggacctttatttatctgaagactggcctgtgtttacttacaaattattaaaagaaacttcatttttctcctccctcagaaccaagaccaagacaccctttttcttgcctattccctttgaagagcagatttatatttaactcagcctttaattttagctatagcccttcctggctttacatgggagctttggttctaactcaccaccttgcagaggcccaaggcttctgtggctgtagaaacacatgctataggttatcagaggcagaagatagctgcccttagggcagcagagagtttcagccttggttactcctttggattcattctcatgtttttagtttggcctctcaggattttacttactctcttgcaagctcaaccataaatttaaaaggattaaaaagtattttttatccaatatttctgttagagtagtttaacatgttttttctgccctaattgctgactaaactttttttttggtacatgtcagaggcaagccgtgactatctaggtatggaaagcatttcttagtgattgatttgtggccattcacaatattgcctaggatctgagtatgtgtatacactcagcctgtatcatgttgaaaattactgtcaatagaacagactaaagtgatctacatgtattaaagaaaactatatattttcaaatttcctaatgtcaataatatccttttgataattcattttaataaaaatagtacaaggtagtttgcaaatgatcaaggaatgttgtaaagaaattgacttttcttcttcagtgtgctttatctagtaactttttttttccag

SEQ ID NO: 25 - DNA, 802 bp, Homo sapiens
  source 1..802: mol_type = other DNA
gtgagttcagttccccaggccaagagcagctgagcggccaggcgcagcctccagagggctctgaagaccccagaggtaccgcgcgccttgtcccccaccccacctcaccccaccctggctttccctcccctccgccctgatgggcctcacaccttcctccgaggatgggggacctgccactggcacaaaaggggcttgacttcagcttctcagagcctgagccctgagggggaggatactgcctgtaaatgcatttccaggggagggcaggcctcccacatccccaggcagcactgtgagttccagagcaggagctcaggcggtggggccagcgtgtgcactgcagcggctgctgtggggcagtgatggcatgggggtctcaacttcctcgggtggcagcacgccctttcctttcctgcacgtctgtcatcagcagcggccttctcatgtggcttctctgggcgtttttgttttctttggggtgtttttgcctcgggttttataccacccttgtggtcggggcttctgggtggaaaatgaggtgttcgctggcttcctgcaatgtggggcttgtccgggcctgggacgctgcttgcatgtggcgctgtgtgcggcagtctgcccggccctgccacctccagggtggccgctctgtgagatgctcattgtcctttttttttgacacttcatcccctgagcctcgaggtgccctgctcatgctaggtaccatgctgaaggttggagggcagagatggcttccctgactttccgagtctctcacattctcttcctcttccctcag

SEQ ID NO: 26 - DNA, 807 bp, Homo sapiens
  source 1..807: mol_type = other DNA
gtgagaggaacatgcttgggcgacgggaagttgaacgcacaaacctgtccagagggcaagatgccccgagccccggggaaggatgaggacacacctgatgtccaggtgtatgggggtgggggcggggactcacacacctgggagacataactgactgtggaagggtcaccgatatcctgggagagagaggcttttaccagagactgggaacatacacccactgatctaactaaggcctggtggggagggcccgaggaagacgaggtgtatgagacggaggaggggagaccccctgaaggaagggggaagaaacgctaaaggaatgtgaaaggccaagcaatggagaacaaacctgaataggggggtgaagacctgagccctggaaagggttcatctcctcagcataaacacgcctgcatggagataagttctaagagagcagactttccctcttcactgttgtattttcattgtcacaaatagtgcctgacacatagtaggcactcactaaatgtctgctgaatgagtgtagaaataatcaccagagtcctggtgaggttgggggcttgtgagaagacaccgtgctttggtgtgcctgtttcaaatgtgcgttttggaggggagaaatacatatatctgaagaaaggaatgatggccaaacgctgggacactcaaaccctgggacagcctttaaagaaatgacagaggaggcctccttcttctagagtgctggccagtgtccagaacttctgtgttgggctttgcagggtgctggggtggagagttttactccaatacctttcctag

SEQ ID NO: 27 - DNA, 208 bp, Homo sapiens
  source 1..208: mol_type = other DNA
gtaaggccagaaagaaaagacaagatccagctcaaagagagaggatggatcttctctctgtcaggaacgggaaagaggaatcagggctaacacacccctatcattgtgtgtctaaattgtaatgtgctcctttcagttgtaattgaattagctcccttctcaaactcacagttcctgctcttcatctgtttttccctctttcctttag

SEQ ID NO: 28 - DNA, 85 bp, Homo sapiens
  source 1..85: mol_type = other DNA
ttggtgtggttggtacgggaactggtgaagagtggggttctgggagccgatggtgtttgtatgacgtttatgaagcagattgcag

SEQ ID NO: 29 - DNA, 114 bp, Homo sapiens
  source 1..114: mol_type = other DNA
ggtacccagttcccaaacctgcactgatctcacttttggagagaggggacatggcttggggcctggaagcacaggatgatcccccagcagagaggaccaaaaacgtctgtaaag

SEQ ID NO: 30 - DNA, 1955 bp, Homo sapiens
  source 1..1955: mol_type = other DNA
gtaggtgaggcagatttagaaccaacaattgtagcgttttatggagcactgtattttaaaatgaatcatgctgggcaggatttaaataaaatagatgatttcacctaataggtaaaatatgaaacgtttctcaccaatcttaactcttaccaacatgtaatcactcagtacgtctcatctgcaatccttgaatccattgttcctttctgagtttcaacattgcctaccttagttgtcctccttggttgggatggatttattcaggagcagtttttgagcaacttgccatgtgctgaacgttgtttaagaagttctggcaggtacaagttatggagacagatgtaaaaagcgaaaaagaaaagtattaagtgctgttgggagtgggacggagaatgtgaaggtaaaatgggccagatcttcccaaaagttggccataaatggggagagctgagtcagataacactaggaagtcatggcatgggaggagctttttgaattcataggaattcagagctgagcacagggcgtgaggtgcctttagaataggcaagtggatggctagagcaggccattgagaaggtgggtggagagtctgagataaaaggcaggtctgaaacagtactctaacacagctgggttgaaacgatccccaggaagaaaaaatgaaggctgaatcttaaccattaagggatatttaatttcagtgagagaaggaagagaaagtagacagtcaaatagatgggggccaccaagagcaaacagtgaaattgaaggcagaggcaggagtttcagtgcaggtggtttgcagagagcaaatacgtggaaaataatgaagctagcacagcacttcagcctcttattctaagaatctgaaaaagaatcatcaagacaattgagaacctgttggtttgttctcacacgcttcctctgtctggtgattattctttttcagactcttggttaccttgtttacttcaatctcagattactctcctctgaacattctccttcggagttcccttactcctttaactcacactcataggatgaacttgattaaccatcacatgagtcccaggtaacccacattaatgttaaaaccaagcccatcatttcatataaaatggattgactttaactcatttaaaatgtcatccaatcttttggaacacaaatgccattttatgggctgtttgtctcctttttgctgataggagaaagaatactctcagtctgggtgtgtctaagtaaactgttcatcccttttcaaatagagagtctaggacagtagtttctctgcatgtttgaggttcagcagttttagggcgtttcctcatcgtctatagtccatgtgtggttctgcagttaagaacctctgaactaggtaatcttacaacatattgggaataatactctctaagcacttctgtgattttctttctctctgtcttgccacaacccagtgtctgtaatgttcagtgtcttaagtcttttgggacatctgcagggtatgagtttttaaaaagaacaacaattcatcctttgcctttgctttctgatactgttctgtttcactttctgattctaagtcttgccagccccgatgtcactacttgttcttaagacctgtgccttctttggcattttgtaggctgtaagtagcatcaattcaaaagatactagctcatgccagggatgaccagacatcattcaaatattagagtagaaaagcttaggaagcagtctcgtgtgttgtgtccactgataccctgttccacctcaatgttctctgcctgtgtgatatttggcactttcactttaacagccttttcttcccagcatgctgatgtgaaagttgaaatgatctcttttaaagccttatcattttcaatgctctattatttctagtatagttctcaggtggcattatttgtgttttctatctcttatgtcag

SEQ ID NO: 31 - DNA, 1103 bp, Homo sapiens
  source 1..1103: mol_type = other DNA
atgttgagaccaacattgacagtgagtccacattaatccagggaatttctgaagaaagagatgggatgatgtcacatggtcagctgaagagtgtccctcagagaactgacttcccagaaacacgtaatgtggaaaagcaccaggacatccccacagtgaaaaatatccaaggaaaggttccaagaatcccctgtgcaaggaaacctttcatatgtgaggagtgtgggaaatccttcagctacttttcttactatgctagacaccagagaatccacactggggagaaaccttttgagtgtagtgagtgtggaaaagcctttaatggtaattcttcgttaattcggcaccagaggattcacactggagagagaccctatcagtgtgaggagtgtgggcgagcctttaatgataatgcaaatctgatcaggcatcagagaatccacagtggggacagaccctattactgtacagagtgtgggaatagtttcacgagtagttccgagtttgttatacatcagagaatccacactggggagaaaccctatgagtgtaatgagtgtggcaaagcttttgttggtaattcacccctacttcggcatcagaaaatccacactggagagaaaccctatgagtgtaatgagtgtggcaaaagctttggaaggacttcccatctaagccaacatcagcgtattcacacaggggaaaagccttattcttgtaaagtatgtggacaagccttcaattttcatacaaaactaactcggcaccagagaattcacagtgaggagaaaccctttgactgtgtagattgtggaaaagccttcagtgctcaggaacaattaaaaaggcatctgagaattcatactcaggagtcttcctatgtatgtgatgagtgtggaaaagccttgactagcaaaagaaatcttcatcagcatcaaagaatccatactggagagaaaccctatgagtgtagcaagtatgagaaggcctttgggacttcttcccagctaggtcaccttgagcatgtctactctggagagaagcctgtgctggacatttgtcgttttggcctcccagaattttttacccccttttactggtaa

SEQ ID NO: 32 - DNA, 10 bp, synthetic construct
  source 1..10: mol_type = other DNA
tttgttccgt

SEQ ID NO: 33 - DNA, 10 bp, synthetic construct
  source 1..10: mol_type = other DNA
gggtggttta

SEQ ID NO: 34 - DNA, 10 bp, synthetic construct
  source 1..10: mol_type = other DNA
gtaggtaggt

SEQ ID NO: 35 - DNA, 10 bp, synthetic construct
  source 1..10: mol_type = other DNA
ttcgttctgc

SEQ ID NO: 36 - DNA, 10 bp, synthetic construct
  source 1..10: mol_type = other DNA
ggtaagtagg

SEQ ID NO: 37 - DNA, 10 bp, synthetic construct
  source 1..10: mol_type = other DNA
ggttagttta

SEQ ID NO: 38 - DNA, 10 bp, synthetic construct
  source 1..10: mol_type = other DNA
ttcgtaggta

SEQ ID NO: 39 - DNA, 10 bp, synthetic construct
  source 1..10: mol_type = other DNA
ggtccactag

SEQ ID NO: 40 - DNA, 10 bp, synthetic construct
  source 1..10: mol_type = other DNA
ttctgttcct

SEQ ID NO: 41 - DNA, 10 bp, synthetic construct
  source 1..10: mol_type = other DNA
tcgttcctta

SEQ ID NO: 42 - DNA, 10 bp, synthetic construct
  source 1..10: mol_type = other DNA
gggatggggt

SEQ ID NO: 43 - DNA, 10 bp, synthetic construct
  source 1..10: mol_type = other DNA
gtttgggggt

SEQ ID NO: 44 - DNA, 10 bp, synthetic construct
  source 1..10: mol_type = other DNA
tatagggggg

SEQ ID NO: 45 - DNA, 10 bp, synthetic construct
  source 1..10: mol_type = other DNA
ggggttggga

SEQ ID NO: 46 - DNA, 10 bp, synthetic construct
  source 1..10: mol_type = other DNA
tttcctgatg

SEQ ID NO: 47 - DNA, 10 bp, synthetic construct
  source 1..10: mol_type = other DNA
tgtttagtta

SEQ ID NO: 48 - DNA, 10 bp, synthetic construct
  source 1..10: mol_type = other DNA
ttcttagtta

SEQ ID NO: 49 - DNA, 10 bp, synthetic construct
  source 1..10: mol_type = other DNA
gttaggtata

SEQ ID NO: 50 - DNA, 10 bp, synthetic construct
  source 1..10: mol_type = other DNA
taatagttta

SEQ ID NO: 51 - DNA, 10 bp, synthetic construct
  source 1..10: mol_type = other DNA
ttcgtttggg